(19) United States
(12) Patent Application Publication
(10) Pub. No.: US 2007/0167631 A1
(43) Pub. Date: Jul. 19, 2007

(54) Novel herbicides

(75) Inventors: Christoph Luthy, Basel (CH); Andrew Edmunds, Basel (CH); Renaud Beaudegnies, Basel (CH); Sebastian Wendeborn, Basel (CH); Jurgen Schaetzer, Basel (CH); William Lutz, Basel (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignees: Christoph LUTHY, Basel (CH); Andrew EDMUNDS, Basel (CH); Renaud BEAUDEGNIES, Basel (CH); Sebastian WENDERBORN, Basel (CH); Jurgen SCHAETZER, Basel (CH); William LUTZ, Basel (CH)

(21) Appl. No.: 10/596,297

(22) PCT Filed: Dec. 10, 2004
(86) PCT No.: PCT/EP04/14113

§ 371 Date: Jun. 8, 2006

(30) Foreign Application Priority Data
Dec. 12, 2003 (CH) 02128/03

(51) Int. Cl. C07D 213/78; C07D 211/72; C07D 211/84; C07D 213/62

(52) U.S. Cl. 546/298

(57) ABSTRACT

Compounds of formula (I) wherein the substituents are as defined in claim 1, and agronomically acceptable salts/N-oxides/isomers/enantiomers of those compounds are suitable for use as herbicides.

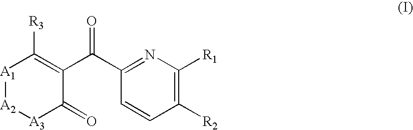

The present invention relates to novel, herbicidally active picolinoylcyclohexanediones, to processes for their preparation, to compositions comprising those compounds, and to their use in controlling weeds, especially in crops of useful plants, or in inhibiting plant growth.

Certain herbicidally active derivatives of picolinic acids disubstituted in the 3,5-position are known, such as, for example from EP-A-O 316 491, the 3,5-substituted picolinoyl derivatives of 1,3-cyclohexanediones substituted in the 2-position.

It has now been found that picolinoylcyclohexanediones that are substituted in the 5,6-position of the pyridine and unsubstituted in the 3,4-position of the pyridine exhibit excellent herbicidal and growth-inhibiting properties.

The present invention accordingly relates to compounds of formula I 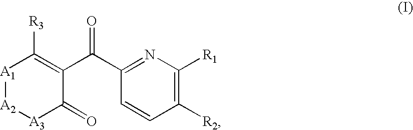
wherein
R₁ is -L₁₀-R₄, -L₁₁-X₁—R₅, —NR₆R₇, —X₂—R₈ or —X₃-L₁-R₉;
L₂, L₄, L₆ and L₈ are each independently of the others C₁-C₄alkylene which may be substituted once, twice or three times by C₁-C₄alkyl, halogen or by C₁-C₄alkoxy and to which C₁-C₄alkylene group there may additionally be spirocyclically bound a C₂-C₅alkylene group, and wherein that C₂-C₅alkylene group may in turn be interrupted once or twice by oxygen, sulfur, sulfinyl or by sulfonyl and/or substituted by C₁-C₄alkyl or by C₁-C₄alkoxy;
L₃, L₅, L₇ and L₉ are each independently of the others C₁-C₄alkylene which may be substituted once, twice or three times by C₁-C₄alkyl, halogen or by C₁-C₄alkoxy;
R₂ is halogen, C₁-C₄haloalkyl, cyano, C₁-C₃haloalkoxy, C₁-C₄alkylthio, C₁-C₄alkylsulfinyl, C₁-C₄alkylsulfonyl, C₁-C₄haloalkylthio, C₁-C₄haloalkylsulfinyl or C₁-C₄haloalkylsulfonyl;
L₁₀ is a direct bond or a C₁-C₆alkylene, C₂-C₆alkenylene or C₂-C₆alkynylene group which may be substituted once, twice or three times by C₁-C₆alkyl, halogen, hydroxy, C₁-C₆alkoxy, C₃-C₆cycloalkyloxy, C₁-C₆alkoxy-C₁-C₆alkoxy, C₁-C₆alkoxy-C₁-C₆alkoxy-C₁-C₆alkoxy or by C₁-C₂alkylsulfonyloxy;
R₄ is halogen, cyano, rhodano, C₁-C₆alkoxycarbonyl, C₃-C₆alkenyloxycarbonyl, C₃-C₆alkynyl-oxycarbonyl, benzyloxycarbonyl, C(O)NR₂₅ₐR₂₆ₐ, formyl, C₁-C₆alkylcarbonyl, C₁-C₆haloalkylcarbonyl, C₁-C₄alkoxy-C₁-C₄alkylcarbonyl, C₁-C₄alkoxy-C₁-C₄alkoxy-C₁-C₄alkylcarbonyl, N—(C₁-C₄alkyl)-C₁-C₄alkylsulfonylamino-C₁-C₄alkylcarbonyl, C₁-C₆haloalkyl, C₂-C₆alkenyl, C₂-C₆-haloalkenyl, C₂-C₆alkynyl, C₂-C₆haloalkynyl, C₃-C₆cycloalkyl, C₁-C₆alkylsulfonyloxy or phenylsulfonyloxy, wherein the phenyl groups may be substituted by one or more C₁-C₆alkyl, C₁-C₆-haloalkyl, C₁-C₆alkoxy, C₁-C₆haloalkoxy, halogen, cyano, hydroxy or nitro groups;
or R₄ is a three- to ten-membered, monocyclic or fused bicyclic ring system which may be aromatic, saturated or partially saturated and which may contain from 1 to 4 hetero atoms selected from nitrogen, oxygen and sulfur, and wherein the ring system may contain not more than 2 oxygen atoms and not more than two sulfur atoms, and each ring system may itself be substituted once, twice or three times by C₁-C₆alkyl, C₁-C₆haloalkyl, C₁-C₄alkoxy-C₁-C₂alkyl, C₂-C₆alkenyl, C₂-C₆haloalkenyl, C₂-C₆alkynyl, C₂-C₆haloalkynyl, C₁-C₆alkoxy, hydroxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di(C₁-C₂alkyl)aminosulfonyl, di(C₁-C₄alkyl)amino, halogen, cyano, nitro, phenyl or by benzylthio, and wherein phenyl and benzylthio may in turn be substituted on the phenyl ring by C₁-C₃alkyl, C₁-C₃haloalkyl, C₁-C₃alkoxy, C₁-C₃haloalkoxy, halogen, cyano or by nitro, and wherein the substituent on the nitrogen in the heterocyclic ring are other than halogen;
or R₄ is hydrogen when L₁₀ is a C₁-C₆alkylene group which may be substituted once, twice or three times by C₁-C₆alkyl or by halogen; or when L₁₀ is a C₂-C₆alkenylene or C₂-C₆alkynylene group which may be substituted once, twice or three times by C₁-C₆alkyl, halogen, hydroxy, C₁-C₆alkoxy, C₃-C₆cycloalkyloxy, C₁-C₆alkoxy-C₁-C₆alkoxy, C₁-C₆alkoxy-C₁-C₆alkoxy-C₁-C₆-alkoxy or by C₁-C₂alkylsulfonyloxy;
R₂₅ₐ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₂₆ₐ is hydrogen, C₁-C₆alkyl, C₃-C₆alkenyl or C₃-C₆alkynyl;
or R₂₅ₐ together with R₂₆ₐ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
L₁₁ is a C₁-C₆alkylene, C₂-C₆alkenylene or C₂-C₆alkynylene group which may be substituted once, twice or three times by halogen, hydroxy, C₁-C₆alkoxy, C₃-C₆cycloalkyloxy, C₁-C₆-alkoxy-C₁-C₆alkoxy, C₁-C₆alkoxy-C₁-C₆alkoxy-C₁-C₆alkoxy or by C₁-C₂alkylsulfonyloxy;
X₁ is oxygen, —OC(O)—, —C(O)—, —C(═NR₁₄ₐ)—, —C(O)O—, —C(O)NR_{14b}—, —OC(O)O—, —N(R₁₀)—O—, —O—NR₁₁—, thio, sulfinyl, sulfonyl, —SO₂NR₁₂—, —NR₁₃SO₂—, —N(SO₂R_{14c})—, —N(R_{14d})C(O)— or —NR₁₄—;
R₁₀, R₁₁, R₁₂, R₁₃, R_{14b}, R_{14d} and R₁₄ are each independently of the others hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl, or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;
R₁₄ₐ is hydroxy, C₁-C₆alkoxy, C₃-C₆alkenyloxy, C₃-C₆alkynyloxy or benzyloxy;
R_{14c} is C₁-C₆alkyl;
R₅ is hydrogen or a C₁-C₈alkyl, C₃-C₈alkenyl or C₃-C₈alkynyl or C₃-C₆cycloalkyl group which may be substituted once, twice or three times by chlorine, bromine, iodine, hydroxy, amino, formyl, nitro, cyano, mercapto, C₁-C₆alkoxy, C₂-C₆alkenyl, C₂-C₆haloalkenyl, C₂-C₆alkynyl, C₂-C₆haloalkynyl, C₃-C₆cycloalkyl, halo-substituted C₃-C₆cycloalkyl, C₃-C₆alkenyloxy, C₃-C₆alkynyloxy, C₁-C₆haloalkoxy, C₃-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆alkoxy, C₁-C₆alkoxycarbonyl, C₁-C₆alkylcarbonyl, phenylcarbonyl, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzyloxy, benzylthio, benzylsulfinyl, benzylsulfonyl, C₁-C₆alkylamino, di(C₁-C₆alkyl)amino, R₁₅ₐC(X₂₃)N(R₁₈ₐ)—, R₁₆ₐN(R₁₇ₐ)C(X₂₄)—, R_{16b}N(R_{17b})C(X₂₅)NR_{18b}—, R_{15c}SO₂N(R_{18c})—, R_{16c}N(R_{17c})C(X₂₆)O—, R_{15b}C(X₂₇)O—, R₁₉R₂₀C═NO—, R₁₅S(O)₂O—, R₁₆N(R₁₇)SO₂—, rhodano, phenyl, phenoxy, phenylthio, phenylsulfinyl or by phenylsulfonyl or which may be substituted from one to seventeen times by fluorine; wherein the phenyl- or benzyl-containing groups may in turn be substituted by one or more C₁-C₆alkyl, C₁-C₆haloalkyl, C₁-C₆alkoxy, C₁-C₆haloalkoxy, halogen, cyano, hydroxy or nitro groups;
R₁₅ₐ, R_{15b} and R_{15c} are hydrogen, C₁-C₆alkyl, C₂-C₆alkenyl, C₃-C₆cycloalkyl, phenyl, benzyl, C₁-C₆alkoxy, C₃-C₆alkenyloxy, C₃-C₆alkynyloxy or benzyloxy, wherein the phenyl groups may be substituted once, twice or three times by C₁-C₆alkyl, C₁-C₆haloalkyl, C₁-C₆alkoxy, C₁-C₆haloalkoxy, halogen, cyano, hydroxy or by nitro;
R₁₆ₐ, R_{16b} and R_{16c} are hydrogen, C₁-C₆alkyl, C₃-C₆alkenyl, C₃-C₆alkynyl, C₃-C₆cycloalkyl or phenyl, wherein phenyl may be substituted once, twice or three times by C₁-C₆alkyl, C₁-C₆haloalkyl, C₁-C₆alkoxy, C₁-C₆haloalkoxy, halogen, cyano, hydroxy or by nitro;
R₁₇ₐ, R_{17b}, R_{17c}, R₁₈ₐ, R_{18b} and R_{18c} are hydrogen, C₁-C₆alkyl, C₃-C₆alkenyl or C₃-C₆alkynyl;
X₂₃, X₂₄, X₂₅, X₂₆ and X₂₇ are oxygen or sulfur;
R₁₅, R₁₆, R₁₇, R₁₉ and R₂₀ are each independently of the others hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl, or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;
or R₅ is a three- to ten-membered monocyclic or fused bicyclic ring system which may be aromatic, saturated or partially saturated and may contain from 1 to 4 hetero atoms selected from nitrogen, oxygen and sulfur, and wherein the ring system is bound to the substituent X₁ directly or via a C₁-C₄alkylene, C₂-C₄alkenylene, C₂-C₄alkynylene, —N(R₁₈)—C₁-C₄alkylene, —O—C₁-C₄alkylene, —S—C₁-C₄alkylene, —S(O)—C₁-C₄alkylene or —SO₂—C₁-C₄alkylene chain, wherein the ring system may not be interrupted by —C(═O)—, —C(═S)—, —C(═NR₅ₐ)—, —N(═O)—, —S(═O)— or by —SO₂—, and each ring system may contain not more than 2 oxygen atoms and not more than two sulfur atoms, and the ring system itself may be substituted once, twice or three times by C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, hydroxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆-alkynylthio, C₂-C₅alkoxyalkylthio, C₃-C₅acetylalkylthio, C₃-C₆alkoxycarbonylalkylthio, C₂-C₄-cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di(C₁-C₂alkyl)aminosulfonyl, di(C₁-C₄alkyl)amino, halogen, cyano, nitro, phenyl or by benzylthio, wherein phenyl and benzylthio may in turn be substituted on the phenyl ring by C₁-C₃alkyl, C₁-C₃haloalkyl, C₁-C₃alkoxy, C₁-C₃-haloalkoxy, halogen, cyano or by nitro, and wherein the substituents on the nitrogen in the heterocyclic ring are other than halogen;
R₅ₐ is C₁-C₆alkyl, hydroxy, C₁-C₆alkoxy, cyano or nitro;
R₁₈ is hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl, or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;
R₆ is hydrogen, C₁-C₆alkyl, C₃-C₆alkenyl, C₃-C₆alkynyl, C₁-C₆haloalkyl, hydroxy, C₁-C₆alkoxy, —C(O)R₁₉ₐ or —C(S)R₂₀ₐ;
R₁₉ₐ and R₂₀ₐ are each independently of the other hydrogen, C₁-C₆alkyl, C₃-C₆cycloalkyl, phenyl, benzyl, heteroaryl, C₁-C₆alkoxy, C₃-C₆alkenyloxy, benzyloxy, C₁-C₄alkylthio or a group NR₂₁R₂₂;
R₂₁ and R₂₂ are each independently of the other hydrogen, C₁-C₆alkyl, C₃-C₆alkenyl, C₃-C₆alkynyl or phenyl, and wherein phenyl, benzyl, benzyloxy and heteroaryl in R₁₉ₐ, R₂₀ₐ, R₂₁ and R₂₂ may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₂₁ together with R₂₂ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₆ is -L₂-X₄—R₂₄; wherein
X₄ is oxygen, —NR₂₃—, —S—, —S(O)— or —S(O)₂—;
R₂₃ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₂₄ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₅)NR₂₅R₂₆, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₂₄ is C(O)—R₇₄ or C(S)—R₇₅;
X₅ is oxygen or sulfur;
R₂₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₂₆ is hydrogen, C₁-C₆alkyl, C₃-C₆alkenyl or C₃-C₆alkynyl;
or R₂₅ together with R₂₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₆ is -L₃-R₂₇;
R₂₇ is formyl, C₁-C₆alkylcarbonyl, C₃-C₆cycloalkylcarbonyl, benzoyl, C₁-C₆alkoxycarbonyl, cyano, C(X₆)NR₂₈R₂₉, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;
   or R₂₇ is C₃-C₆cycloalkyl or C₅-C₆cycloalkenyl each of which may in turn be substituted once, twice or three times by C₁-C₄alkyl, halogen or by C₁-C₄alkoxy;
   X₆ is oxygen or sulfur;
   R₂₈ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₂₉ is hydrogen, C₁-C₆alkyl, C₃-C₆alkenyl or C₃-C₆alkynyl;
   or R₂₈ together with R₂₉ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₇ is hydrogen, C₁-C₆alkyl, C₃-C₆alkenyl, C₃-C₆alkynyl, C₁-C₆haloalkyl, C₃-C₆cycloalkyl, phenyl, benzyl, heteroaryl, C(X₇)R₃₀ or NR₃₃R₃₄, wherein phenyl, benzyl and heteroaryl may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   X₇ is oxygen or sulfur;
   R₃₀ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₃₁R₃₂;
   R₃₁ and R₃₃ are each independently of the other hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₃₂ and R₃₄ are each independently of the other hydrogen, C₁-C₆alkyl, C₃-C₆alkenyl or C₃-C₆alkynyl;
   or R₃₁ together with R₃₂ or R₃₃ together with R₃₄, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₇ is -L₄-X₈—R₃₅; wherein
   X₈ is oxygen, —NR₃₆—, —S—, —S(O)— or —S(O)₂—;
   R₃₆ is hydrogen, C₁-C₆alkoxy, C₃-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₃₅ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₉)NR₃₇R₃₈, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   X₉ is oxygen or sulfur;
   R₃₇ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₃₈ is hydrogen, C₁-C₆alkyl, C₃-C₆alkenyl or C₃-C₆alkynyl;
   or R₃₇ together with R₃₈ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₇ is -L₅-R₃₉;
   R₃₉ is formyl, C₁-C₆alkylcarbonyl, C₃-C₆cycloalkylcarbonyl, benzoyl, C₁-C₆alkoxycarbonyl, cyano, C(X₁₀)NR₄₀R₄₁, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;
   or R₃₉ is C₃-C₆cycloalkyl or C₅-C₆cycloalkenyl each of which may in turn be substituted once, twice or three times by C₁-C₄alkyl, halogen or by C₁-C₄alkoxy;
   X₁₀ is oxygen or sulfur;
   R₄₀ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₄₁ is hydrogen, C₁-C₆alkyl, C₃-C₆alkenyl or C₃-C₆alkynyl;
   or R₄₀ together with R₄₁ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₆ and R₇ together with the nitrogen atom to which they are bonded form a carbocyclic 3- to 7-membered, saturated or partially saturated or unsaturated monocyclic or bicyclic ring system which may be interrupted once by oxygen, once by sulfur, from one to three times by nitrogen and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃-haloalkylthio, cyano, nitro or by C₁-C₄alkoxycarbonyl; wherein each ring system may not be interrupted by —C(═O)—, —C(═S)—, —C(═NR₅ₐ)—, —N(═O)—, —S(═O)— or by —SO₂—;
   R₅ₐ is C₁-C₆alkyl, hydroxy, C₁-C₆alkoxy, cyano or nitro;
   X₂ is oxygen, —NR₄₂—, sulfur, —S(O)— or —S(O)₂—;
   R₄₂ is hydrogen, C₁-C₆alkyl, C₃-C₆alkenyl, C₃-C₆alkynyl, C₁-C₆haloalkyl, C₃-C₆cycloalkyl, phenyl, heteroaryl, C(X₁₁)R₄₃ or NR₄₆R₄₇;
   X₁₁ is oxygen or sulfur;
   R₄₃ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₄₄R₄₅;
   R₄₄ and R₄₆ are each independently of the other hydrogen, C₁-C₅alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₄₅ and R₄₇ are each independently of the other hydrogen, C₁-C₆alkyl, C₃-C₆alkenyl or C₃-C₆alkynyl;
   or R₄₄ together with R₄₅ or F₄₆ together with R₄₇, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₄₂ is -L₆-X₁₂—R₄₈; wherein
   X₁₂ is oxygen, —NR₄₉—, —S—, —S(O)— or —S(O)₂—;
   R₄₉ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₄₈ is a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆alkylsulfonyl, cyano, C(X₁₃)NR₅₀R₅₁, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   X₁₃ is oxygen or sulfur;
   R₅₀ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₅₁ is hydrogen, C₁-C₆alkyl, C₃-C₆alkenyl or C₃-C₆alkynyl;
   or R₅₀ together with R₅₁ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino; or R₄₂ is -L₇-R₅₂;
R₅₂ is formyl, C₁-C₆alkylcarbonyl, C₃-C₆cycloalkylcarbonyl, benzoyl, C₁-C₆alkoxycarbonyl, cyano, C(X₁₄)NR₅₃R₅₄, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;
   or R₅₂ is C₃-C₆cycloalkyl or C₅-C₆cycloalkenyl each of which may in turn be substituted once, twice or three times by C₁-C₄alkyl, halogen or by C₁-C₄alkoxy;
   X₁₄ is oxygen or sulfur;
   R₅₃ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₅₄ is hydrogen, C₁-C₆alkyl, C₃-C₆alkenyl or C₃-C₆alkynyl;
   or R₅₃ together with R₅₄ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₈ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₁₅)NR₅₅R₅₆, C₃-C₆cycloalkyl, phenyl, phenoxy or by 5- or 6-membered heteroaryl or heteroaryloxy, and wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   X₁₅ is oxygen or sulfur;
   R₅₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₅₆ is hydrogen, C₁-C₆alkyl, C₃-C₆alkenyl or C₃-C₆alkynyl;
   or R₅₅ together with R₅₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₈ is cyano, C(O)—R₇₆ or C(S)—R₇₇;
   X₃ is oxygen, —NR₅₇—, sulfur, —S(O)— or —S(O)₂—;
   R₅₇ is hydrogen, C₁-C₆alkyl, C₃-C₆alkenyl, C₃-C₆alkynyl, C₁-C₆haloalkyl, C₃-C₆cycloalkyl, phenyl, heteroaryl, C(X₁₆)R₅₈ or NR₆₁R₆₂;
   X₁₆ is oxygen or sulfur;
   R₅₈ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₅₉R₆₀;
   R₅₉ and R₆₁ are each independently of the other hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₆₀ and R₆₂ are each independently of the other hydrogen, C₁-C₆alkyl, C₃-C₆alkenyl or C₃-C₆alkynyl;
   or R₅₉ together with R₆₀ or R₆₁ together with R₆₂, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₅₇ is -L₈-X₁₇—R₆₃; wherein
   X₁₇ is oxygen, —NR₆₄—, —S—, —S(O)— or —S(O)₂—;
   R₆₄ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₆₃ is a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆alkylsulfonyl, cyano, C(X₁₈)NR₆₅R₆₆, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   X₁₈ is oxygen or sulfur;
   R₆₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₆₆ is hydrogen, C₁-C₆alkyl, C₃-C₆alkenyl or C₃-C₆alkynyl;
   or R₆₅ together with R₆₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₅₇ is -L₉-R₆₇;
   R₆₇ is formyl, C₁-C₆alkylcarbonyl, C₃-C₆cycloalkylcarbonyl, benzoyl, C₁-C₆alkoxycarbonyl, cyano, C(X₁₉)NR₆₈R₆₉, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl; or R₆₇ is C₃-C₆cycloalkyl or C₅-C₆cycloalkenyl each of which may in turn be substituted once, twice or three times by C₁-C₄alkyl, halogen or by C₁-C₄alkoxy;
   X₁₉ is oxygen or sulfur;
   R₆₈ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₆₉ is hydrogen, C₁-C₆alkyl, C₃-C₆alkenyl or C₃-C₆alkynyl;
   or R₆₈ together with R₆₉ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   L₁ is C₁-C₄alkylene which may be substituted once, twice or three times by C₁-C₄alkyl, halogen or by C₁-C₄alkoxy and to which C₁-C₄alkylene group there may be spirocyclically bound a further C₂-C₅alkylene group which may in turn be interrupted once or twice by oxygen, sulfur, sulfinyl or by sulfonyl and/or substituted by C₁-C₄alkyl or by C₁-C₄alkoxy;
   or L₁ is C₁-C₄alkylene which may be substituted once, twice or three times by C₁-C₄alkyl, halogen or by C₁-C₄alkoxy, and wherein a carbon atom of that C₁-C₄alkylene group together with R₉ or with R₇₀ forms, by means of a further C₂-C₆alkylene chain, a ring system which may additionally be interrupted once or twice by oxygen, sulfur, sulfinyl or by sulfonyl and/or substituted by C₁-C₄alkyl or by C₁-C₄alkoxy; R₉ is a group —X₂₀—R₇₀, wherein
   X₂₀ is oxygen, —NR₇₁—, —S—, —S(O)— or —S(O)₂—;
   R₇₁ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₂₁)NR₇₂R₇₃, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   X₂₁ is oxygen or sulfur;
   R₇₂ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₇₃ is hydrogen, C₁-C₆alkyl, C₃-C₆alkenyl or C₃-C₆alkynyl;
   or R₇₂ together with R₇₃ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₇₀ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₁₅ₐ)NR₅₅ₐR₅₆ₐ, C₃-C₆cycloalkyl, phenyl, phenoxy or by 5- or 6-membered heteroaryl or heteroaryloxy, and wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   X₁₅ₐ is oxygen or sulfur;
   R₅₅ₐ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₅₆ₐ is hydrogen, C₁-C₆alkyl, C₃-C₆alkenyl or C₃-C₆alkynyl;
   or R₅₅ₐ together with R₅₆ₐ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₇₀ is C₁-C₁₀alkylideneimino, (phenyl-C₁-C₄alkylidene)imino, or phenyl, wherein phenyl may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   or R₇₀ is cyano, C(O)—R₇₈ or C(S)—R₇₉;
   or R₉ is formyl, C₁-C₆alkylcarbonyl, C₃-C₆cycloalkylcarbonyl, benzoyl, C₁-C₆alkoxycarbonyl, cyano, C(X₃₅)NR₁₂₅R₁₂₆, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;
   or R₉ is C₃-C₆cycloalkyl or C₅-C₆cycloalkenyl each of which may in turn be substituted once, twice or three times by C₁-C₄alkyl, halogen or by C₁-C₄alkoxy;
   X₃₅ is oxygen or sulfur;
   R₁₂₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₁₂₆ is hydrogen, C₁-C₆alkyl, C₃-C₆alkenyl or C₃-C₆alkynyl;
   or R₁₂₅ together with R₁₂₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₇₄, R₇₅, R₇₆, R₇₇, R₇₈ and R₇₉ are each independently of the others hydrogen, C₁-C₆alkyl, C₃-C₆cycloalkyl, phenyl, benzyl, heteroaryl, C₁-C₆alkoxy, C₃-C₆alkenyloxy, benzyloxy, C₁-C₄alkylthio or NR₁₂₇R₁₂₈, wherein phenyl, benzyl or heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₁₂₇ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₁₂₈ is hydrogen, C₁-C₆alkyl, C₃-C₆alkenyl or C₃-C₆alkynyl;
   or R₁₂₇ together with R₁₂₈ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
   R₃ is hydroxy, O⁻M⁺ wherein M⁺ is a metal cation or an ammonium cation, or is halogen or S(O)ₚR₈₀, wherein
   p is 0, 1 or 2;
   R₈₀ is C₁-C₁₂alkyl, C₂-C₁₂alkenyl, C₂-C₁₂alkynyl, C₃-C₁₂alkenyl, C₃-C₁₂cycloalkyl or C₅-C₁₂-cycloalkenyl;
   or R₈₀ is R₁₂₁—C₁-C₁₂alkylene or R₁₂₂—C₂-C₁₂alkenylene, wherein the alkylene or alkenylene chain may be interrupted by —O—, —S—, —S(O)—, —SO₂— or by —C(O)— and/or substituted from one to five times by R₁₂₃;
   or R₈₀ is phenyl which may be substituted once, twice, three times, four times or five times by R₁₂₄;
   R₁₂₁ and R₁₂₂ are each independently of the other halogen, cyano, rhodano, hydroxy, C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, C₂-C₆alkenylthio, C₂-C₆alkynylthio, C₁-C₆alkylsulfonyloxy, phenylsulfonyloxy, C₁-C₆-alkylcarbonyloxy, benzoyloxy, C₁-C₄alkoxycarbonyloxy, C₁-C₆alkylcarbonyl, C₁-C₄alkoxycarbonyl, benzoyl, aminocarbonyl, C₁-C₄alkylaminocarbonyl, C₃-C₆cycloalkyl, phenyl, phenoxy, phenylthio, phenylsulfinyl or phenylsulfonyl, wherein the phenyl-containing groups may in turn be substituted once, twice or three times by halogen, C₁-C₃alkyl, C₁-C₃haloalkyl, hydroxy, C₁-C₃alkoxy, C₁-C₃haloalkoxy, cyano or by nitro;
   R₁₂₃ is hydroxy, halogen, C₁-C₆alkyl, C₁-C₆alkoxy, C₁-C₆alkylthio, C₁-C₆alkylsulfinyl, C₁-C₆alkylsulfonyl, cyano, carbamoyl, carboxy, C₁-C₄alkoxycarbonyl or phenyl, wherein phenyl may be substituted once, twice or three times by hydrogen, C₁-C₄alkyl, C₁-C₄haloalkyl, C₃-C₄alkenyl, C₃-C₄alkynyl or by C₁-C₄alkoxy;
   R₁₂₄ is halogen, C₁-C₃alkyl, C₁-C₃haloalkyl, hydroxy, C₁-C₃alkoxy, C₁-C₃haloalkoxy, cyano or nitro;
   A₁ is —C(R₁₁₂R₁₁₃)— or —NR₁₁₄—;
   A₂ is —C(R₁₁₅R₁₁₆)ₘ—, —C(═O)—, —O—, —NR₁₁₇— or —S(O)_{q}—;
   A₃ is —C(R₁₁₈R₁₁₉)— or —NR₁₂₀—;
   with the proviso that A₂ is other than —O— or —S(O)_{q}— when A₁ is —NR₁₁₄— and/or A₃ is —NR₁₂₀;
   R₁₁₂ and R₁₁₈ are each independently of the other hydrogen, C₁-C₄alkyl, C₂-C₄alkenyl, C₂-C₄alkynyl, C₁-C₄alkylthio, C₁-C₄alkylsulfinyl, C₁-C₄alkylsulfonyl, C₁-C₄alkoxycarbonyl, hydroxy, C₁-C₄alkoxy, C₃-C₄alkenyloxy, C₃-C₄alkynyloxy, hydroxy-C₁-C₄alkyl, C₁-C₄alkylsulfonyloxy-C₁-C₄alkyl, halogen, cyano or nitro;
   R₁₁₃ and R₁₁₉ are each independently of the other hydrogen, C₁-C₄alkyl or C₁-C₄alkylthio, C₁-C₄alkylsulfinyl or C₁-C₄alkylsulfonyl;
   or R₁₁₃ together with R₁₁₂ and/or R₁₉ together with R₁₁₈ form a C₂-C₅alkylene chain which may be interrupted by —O—, —C(O)O— or by —S(O)ᵣ—;
   R₁₁₄ and R₁₂₀ are each independently of the other hydrogen, C₁-C₄alkyl, C₁-C₄haloalkyl, C₃-C₄alkenyl, C₃-C₄alkynyl or C₁-C₄alkoxy;
   R₁₁₅ is hydrogen, hydroxy, C₁-C₄alkyl, C₁-C₄haloalkyl, C₁-C₃hydroxyalkyl, C₁-C₄alkoxy-C₁-C₃alkyl, C₁-C₄alkylthio-C₁-C₃alkyl, C₁-C₄alkylcarbonyloxy-C₁-C₃alkyl, C₁-C₄alkylsulfonyloxy-C₁-C₃alkyl, tosyloxy-C₁-C₃alkyl, di(C₁-C₄alkoxy)C₁-C₃alkyl, C₁-C₄alkoxycarbonyl, formyl, C₃-C₅oxacycloalkyl, C₃-C₅thiacycloalkyl, C₃-C₄dioxacycloalkyl, C₃-C₄dithiacycloalkyl, C₃-C₄-oxathiacycloalkyl, C₁-C₄alkoxyiminomethyl, cyano, carbamoyl, C₁-C₄alkylaminocarbonyl or di(C₁-C₄alkyl)aminocarbonyl;
   or R₁₁₅ together with R₁₁₂ or R₁₁₃ or R₁₁₄ or R₁₁₆ or R₁₁₈ or R₁₁₉ or R₁₂₀ or, when m is 2, also with a second R₁₁₅ form a C₁-C₄alkylene bridge;
   R₁₁₆ is hydrogen, C₁-C₃alkyl or C₁-C₃haloalkyl;
   R₁₁₇ is hydrogen, C₁-C₃alkyl, C₁-C₃haloalkyl, C₁-C₄alkoxycarbonyl, C₁-C₄alkylcarbonyl or di(C₁-C₄alkyl)aminocarbonyl;
   m is 1 or 2; and
   q and r are each independently of the other 0, 1 or 2;
   and also to agronomically acceptable salts, tautomers, isomers and enantiomers of those compounds.

The alkyl groups in the substituent definitions may be straight-chained or branched and are, for example, methyl, ethyl, n-propyl, isopropyl, n-butyl, sec-butyl, isobutyl, tert-butyl, pentyl, hexyl, heptyl and octyl and branched isomers thereof. Alkoxy, alkylthio, alkenyl and alkynyl radicals are derived from the mentioned alkyl radicals. The alkenyl and alkynyl groups may be mono- or poly-unsaturated, in which case an allenyl group and a mixed alkene-alkynyl group are also included.

Alkoxy groups are accordingly methoxy, ethoxy, n-propoxy, isopropoxy, n-butoxy, sec-butoxy, isobutoxy, tert-butoxy.

Alkylthio groups and oxidised forms thereof preferably have a chain length of from 1 to 3 carbon atoms; preference is given to, for example, methylthio, ethylthio, n-propylthio and isopropylthio; especially methyl- and ethyl-thio. Alkylsulfinyl is, for example, methylsulfinyl, ethylsulfinyl, n-propylsulfinyl or isopropylsulfinyl, and alkylsulfonyl is preferably methylsulfonyl, ethylsulfonyl, propylsulfonyl or isopropylsulfonyl; preferably methylsulfonyl or ethylsulfonyl.

Halogen is generally fluorine, chlorine, bromine or iodine; preferably fluorine, chlorine or bromine. Halo-substituted alkyl groups, that is to say haloalkyl groups, preferably have a chain length of from 1 to 6 carbon atoms. C₁-C₄Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,2-trichloroethyl, 1,1,2,2-tetrafluoroethyl, 2,2,3,3-tetrafluoropropyl, 2,2,3,3,3-pentafluoropropyl or 2,2,3,4,4,4-hexafluorobutyl; preferably fluoromethyl, difluoromethyl, difluorochloromethyl, dichlorofluoromethyl, trifluoromethyl, 2-chloroethyl, 2,2,2-trifluoroethyl, 2,2,3,3-tetrafluoropropyl or 2,2,3,3,3-pentafluoropropyl.

As haloalkenyl groups, alkenyl groups substituted once or more than once by halogen are suitable, halogen being especially fluorine or chlorine, for example 2, 2-difluoro-1-methylvinyl, 3-fluoropropenyl, 3-chloropropenyl, 3-bromopropenyl, 2,3,3-trifluoropropenyl, 2,3,3-trichloro-propenyl or 4,4,4-trifluoro-but-2-en-1-yl. As haloalkynyl, for example alkynyl groups substituted once or more than once by halogen are suitable, halogen being bromine or iodine or also fluorine or chlorine, for example 3-fluoropropynyl, 3-chloropropynyl, 3-bromopropynyl, 3,3,3-trifluoropropynyl or 4,4,4-trifluoro-but-2-yn-1-yl. The same is also correspondingly true for halogen in association with other definitions such as haloalkoxy, haloalkylthio, haloalkylsulfinyl, haloalkylsulfonyl or halophenyl.

The definition of R₄ or R₅ as a three- to ten-membered monocyclic or fused bicyclic ring system which may be aromatic, saturated or partially saturated and which may contain from 1 to 4 hetero atoms selected from nitrogen, oxygen and sulfur, and wherein the ring system may contain not more than 2 oxygen atoms and not more than two sulfur atoms, and the ring system may itself be substituted, is to be understood in the context of the present invention as meaning especially ring systems in which two oxygen atoms and/or one oxygen atom and one sulfur atom are not located next to one another, such as, for example, phenyl, naphthyl, indenyl, cyclopentenyl, cyclohexenyl or C₃-C₈cycloalkyl, C₃-C₆oxacycloalkyl, C₂-C₅dioxacycloalkyl or similar ring systems, such as especially aromatic five- or six-membered heteroaryl groups containing from 1 to 4 hetero atoms selected from nitrogen, oxygen and sulfur, and partially or fully saturated three- to six-membered ring systems which especially contain 1 or 2 oxygen atoms, such as, for example, oxiranyl, oxetan-3-yl, tetrahydrofuran-3-yl, tetrahydropyran-2-yl, 1,3-dioxacyclopent-2-yl, 1,3-dioxacyclopent-4-yl, 1,3-dioxacyclohex-2-yl, 1,3-dioxacyclohex-5-yl or cis- and/or trans-1,3-dioxa-4,5-dimethyl-cyclopent-2-yl, or fully saturated three- to six-membered ring systems that contain 1 or 2 sulfur atoms, such as, for example, 1,3-oxathio-cyclopent-2-yl, tetrahydrothien-2-yl, 1,3-oxathio-cyclopent-2-yl or 1,3-dithiacyclopent-2-yl.

Heteroaryl, such as, for example, in the definition of R₆, R₇, R₈ or R₉ or in the case of a five- or six-membered, monocyclic or fused bicyclic, aromatic ring system R₄ or R₅, is understood to be especially an aromatic 5- or 6-membered heteroaryl group bonded via a carbon atom, which group may be interrupted once by oxygen, once by sulfur and/or once, twice or three times by nitrogen, for example 1-methyl-1H-pyrazol-3-yl, 1-ethyl-1H-pyrazol-3-yl, 1-propyl-1H-pyrazol-3-yl, 1H-pyrazol-3-yl, 1,5-dimethyl-1H-pyrazol-3-yl, 4-chloro-1-methyl-1H-pyrazol-3-yl, 3-isoxazolyl, 5-methyl-3-isoxazolyl, 3-methyl-5-isoxazolyl, 5-isoxazolyl, 1H-pyrrol-2-yl, 1-methyl-1H-pyrrol-2-yl, 1-methyl-1H-pyrrol-3-yl, 2-furyl, 5-methyl-2-furyl, 3-furyl, 5-methyl-2-thienyl, 2-thienyl, 3-thienyl, 1-methyl-1H-imidazol-2-yl, 1H-imidazol-2-yl, 1-methyl-1H-imidazol-4-yl, 1-methyl-1H-imidazol-5-yl, 4-methyl-2-oxazolyl, 5-methyl-2-oxazolyl, 2-oxazolyl, 2-methyl-5-oxazolyl, 2-methyl-4-oxazolyl, 4-methyl-2-thiazolyl, 5-methyl-2-thiazolyl, 2-thiazolyl, 2-methyl-5-thiazolyl, 2-methyl-4-thiazolyl, 3-methyl-4-isothiazolyl, 3-methyl-5-isothiazolyl, 5-methyl-3-isothiazolyl, 1-methyl-1H-1,2,3-triazol-4-yl, 2-methyl-2H-1,2,3-triazol-4-yl, 4-methyl-2H-1,2,3-triazol-2-yl, 1-methyl-1H-1,2,4-triazol-3-yl, 1,5-dimethyl-1H-1,2,4-triazol-3-yl, 4,5-dimethyl-4H-1,2,4-triazol-3-yl, 4-methyl-4H-1,2,4-triazol-3-yl, 5-methyl-1,2,3-oxadiazol-4-yl, 1,2,3-oxadiazol-4-yl, 3-methyl-1,2,4-oxadiazol-5-yl, 5-methyl-1,2,4-oxadiazol-3-yl, 5-methyl-1,2,3-thiadiazol-4-yl, 1,2,3-thiadiazol-4-yl, 3-methyl-1,2,4-thiadiazol-5-yl, 5-methyl-1,2,4-thiadiazol-3-yl, 4-methyl-1,2,5-thiadiazol-3-yl, 5-methyl-1,3,4-thiadiazol-2-yl, 2-pyridyl, 6-methyl-2-pyridyl, 4-pyridyl, 3-pyridyl, 6-methyl-3-pyridazinyl, 5-methyl-3-pyridazinyl, 3-pyridazinyl, 4,6-dimethyl-2-pyrimidinyl, 4-methyl-2-pyrimidinyl, 2-pyrimidinyl, 2-methyl-4-pyrimidinyl, 2-chloro-4-pyrimidinyl, 2,6-dimethyl-4-pyrimidinyl, 4-pyrimidinyl, 2-methyl-5-pyrimidinyl, 6-methyl-2-pyrazinyl, 2-pyrazinyl, 4,6-dimethyl-1,3,5-triazin-2-yl, 4,6-dichloro-1,3,5-triazin-2-yl, 1,3,5-triazin-2-yl, 4-methyl-1,3,5-triazin-2-yl, 3-methyl-1,2,4-triazin-5-yl or 3-methyl-1,2,4-triazin-6-yl. A heteroaryl group bonded via the N atom is understood to be, for example, 1H-pyrrol-1-yl, 1H-pyrazol-1-yl, 3-methyl-1H-pyrazol-1-yl, 3,5-dimethyl-1H-pyrazol-1-yl, 3-trifluoromethyl-1H-pyrazol-1-yl, 3-methyl-1H-1,2,4-triazol-1-yl, 5-methyl-1H-1,2,4-triazol-1-yl or 4H-1,2,4-triazol-4-yl.

The definition of L₁ as C₁-C₄alkylene which may be substituted once, twice or three times by C₁-C₄alkyl, halogen or by C₁-C₄alkoxy, wherein a carbon atom of the L₁ chain forms together with R₉ a C₂-C₆alkylene chain which may be interrupted once or twice by oxygen, sulfur, sulfinyl or by sulfonyl and may be substituted by C₁-C₄alkyl or by C₁-C₄alkoxy, is to be understood in the context of the present invention as being, for example, the following cyclic C₂-C₆alkylene groups: C₃-C₆oxacycloalkyl, C₂-C₅dioxacycloalkyl, C₃-C₆oxacycloalkyl-C₁-C₂-alkyl, C₃-C₆dioxacycloalkyl-C₁-C₂alkyl or similar oxygen- or sulfur-containing groups, especially a C₃-C₆cycloalkyl group interrupted once or twice by oxygen, such as, for example, oxetan-3-yl, tetrahydrofuran-3-yl, tetrahydropyran-2-yl, 1,3-dioxacyclohex-4-yl, or 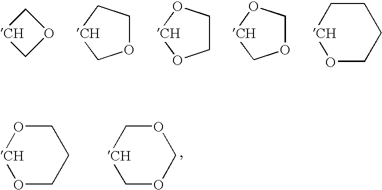
wherein in each case the marked ′C atom is bonded to X₃; or oxiranyl-methyl, 3-oxetanyl-methyl, tetrahydrofuran-3-yl-methyl, tetrahydrofuran-2-yl-methyl, 1,3-dioxacyclopent-3-yl-methyl, 1,3-dioxa-4,5-dimethyl-cyclopent-3-yl-methyl, 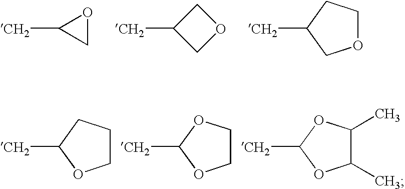
or a C₃-C₆cycloalkyl group interrupted once or twice by sulfur, e.g. 1,3-oxathio-cyclopent-2-yl, tetrahydrothien-2-yl-methyl, 1,3-oxathio-cyclopent-2-yl-methyl, 1,3-dithiacyclopent-2-yl-methyl, 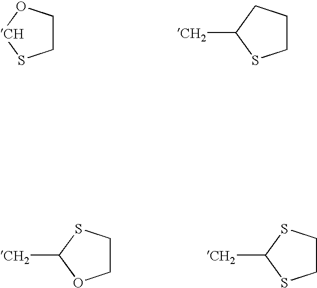
etc., wherein in each case the marked ′C atom is bonded to X₁.

The definition of NR₆R₇ wherein R₆ together with R₇ and the common N atom forms a carbocyclic 3- to 7-membered, saturated or partially saturated monocyclic or bicyclic ring system is to be understood as meaning, for example, morpholino (=morpholin-4-yl), cis- and/or trans-2,6-dimethylmorpholin-4-yl, thiomorpholin-4-yl, N-methyl-piperidin-1-yl, 1H-pyrrol-1-yl, 1H-pyrazol-1-yl, 3-methyl-1H-pyrazol-1-yl, 3,5-dimethyl-1H-pyrazol-1-yl, 3-trifluoromethyl-1H-pyrazol-1-yl, 3-methyl-1H-1,2,4-triazol-1-yl, 5-methyl-1H-1,2,4-triazol-1-yl, 4H-1,2,4-triazol-4-yl, or groups according to the formulae 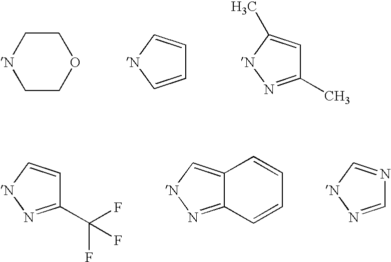
etc., wherein in each case the marked ′N atom is bonded to the picolinyl group.

The definition of L₁, L₂, L₄, L₆ and L₈ as C₁-C₄alkylene, to which C₁-C₄alkylene group there may be spirocyclically bound a C₂-C₅alkylene group, is to be understood as meaning, for example, a C₁-C₃alkylene chain that contains a cyclopropyl group or that is substituted by a 1,3-dioxolan-2-yl group, such as, for example, 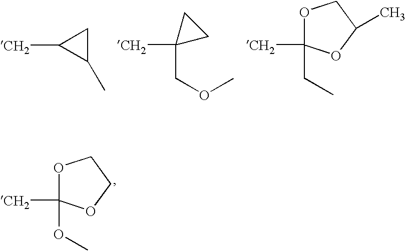
wherein in each case the marked ′C atom represents the left-hand side valence of the definitions containing the respective substituent L. For example, in —X₃-L₁-R₉ the marked ′C atom is linked to the substituent X₃. In general, such alkylene chains, for example C₁-C₄alkylene for L₁ and L₄, can also be substituted by one or more C₁-C₃alkyl groups, especially by methyl groups. Such alkylene chains and the alkylene chains and groups interrupted by oxygen or by sulfur are preferably unsubstituted. Preferably, groups containing C₃-C₆cycloalkyl, oxiranyl, oxetanyl, C₃-C₅oxacycloalkyl, C₃-C₅thiacycloalkyl, C₃-C₄dioxacycloalkyl, C₃-C₄dithiacycloalkyl or C₃-C₄oxathiacycloalkyl and also the groups A₁, A₂ and A₃ are also unsubstituted.

In the case that a chemical group is substituted more than once by substituents listed in a list of substituents, such as for example L₂ in the meaning of C₁-C₄alkylene which is substituted twice or three times by C₁-C₄alkyl, halogen or by C₁-C₄alkoxy, said chemical group can be substituted by different substituents from said list of substituents. The same applies mutatis mutandis for ring systems that are substituted more than once, such as for example R₅ in the meaning of a three- to ten-membered ring system, which is substituted twice or three times by C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, hydroxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆-alkynylthio, C₂-C₅alkoxyalkylthio, C₃-C₅acetylalkylthio, C₃-C₆alkoxycarbonylalkylthio, C₂-C₄-cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di(C₁-C₂alkyl)aminosulfonyl, di(C₁-C₄alkyl)amino, halogen, cyano, nitro, phenyl or by benzylthio.

The compounds of formula I may occur in various tautomeric forms, as shown by way of example for compounds of formula I wherein R₃ is hydroxy by formulae I′, I″, I′″ and I″″, the forms I″ and I″″ being preferred as isolated forms and formula I″″ also representing a rota-meric form of I″.

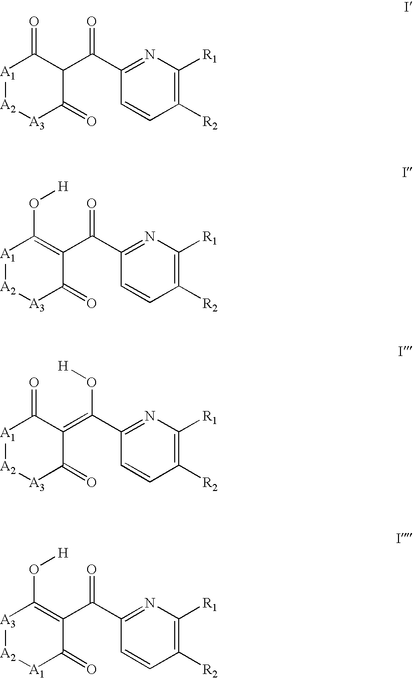

When there is a C═C or C═N double bond in compounds of formula I, such as, especially, in the groups R₁, the compounds of formula I may, when asymmetry exists, be in the ‘E’ or in the ‘Z’ form. If a further asymmetric centre is present, for example an asymmetric carbon atom in the group R₁, or as a result of the spatial arrangement of A₁, A₂, A₃ and the substituents R₁₁₂, R₁₁₃, R₁₁₅, R₁₁₆, R₁₁₈ and R₁₁₉, chiral ‘R’ and ‘S’ forms and/or constitutional isomeric forms may also occur. The present invention accordingly includes also all those stereoisomeric and tautomeric forms of the compound of formula I.

The invention relates also to the salts which the compounds of formula I are able to form preferably with amines, alkali metal and alkaline earth metal cations or quaternary ammonium bases. Suitable salt formers are described, for example, in WO 98/41089. Among the alkali metal and alkaline earth metal hydroxides as salt formers, special mention may be made of the hydroxides of lithium, sodium, potassium, magnesium and calcium, but especially the hydroxides of sodium and potassium.

Examples of amines suitable for ammonium salt formation include ammonia as well as primary, secondary and tertiary C₁-C₁₈alkylamines, C₁-C₄hydroxyalkylamines and C₂-C₄alkoxyalkylamines, for example methylamine, ethylamine, n-propylamine, isopropylamine, the four butylamine isomers, n-amylamine, isoamylamine, n-hexylamine, heptylamine, octylamine, nonylamine, decylamine, pentadecylamine, hexadecylamine, heptadecyl-amine, octadecylamine, methyl-ethylamine, methyl-isopropylamine, methyl-hexylamine, methyl-nonylamine, methyl-pentadecylamine, methyl-octadecylamine, ethyl-butylamine, ethyl-heptylamine, ethyl-octylamine, hexyl-heptylamine, hexyl-octylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, benzidines, naphthylamines and o-, m- and p-chloroanilines; but especially triethylamine, isopropylamine and diisopropylamine.

Preferred quaternary ammonium bases suitable for salt formation correspond, for example, to the formula [⁺N(RₐR_{b}R_{c}R_{d})⁻OH] wherein Rₐ, R_{b}, R_{c} and R_{d} are each independently of the others C₁-C₄alkyl. Other suitable tetraalkylammonium bases with other anions can be obtained, for example, by anion exchange reactions.

In preferred compounds of formula I:

a) A₁ is —C(R₁₁₂R₁₁₃)—, A₂ is —C(R₁₁₅R₁₁₆)—, A₃ is —C(R₁₁₈R₁₁₉)—, R₁₁₂ is hydrogen, methyl, methoxy, methylthio, methylsulfinyl, methylsulfonyl, methoxycarbonyl or ethoxycarbonyl and R₁₁₃, R₁₁₅, R₁₁₆, R₁₁₈ and R₁₁₉ are each independently of the others hydrogen or methyl; especially preferably A₁, A₂ and A₃ are unsubstituted methylene;

b) A₁ is —C(R₁₁₂R₁₁₃)—, R₁₁₂ together with R₁₁₃ ethylene is a spirocyclic 3-membered ring, A₂ is —C(R₁₁₅R₁₁₆)—, A₃ is C(R₁₁₈R₁₁₉)— and R₁₁₅, R₁₁₆, R₁₁₈ and R₁₁₉ are each independently of the others hydrogen or methyl; especially A₂ and A₃ are unsubstituted methylene;

c) A₁ is —C(R₁₁₂R₁₁₃)—, A₃ is —C(R₁₁₈R₁₁₉)—, R₁₁₂, R₁₁₃, R₁₁₈ and R₁₁₉ are each methyl and A₂ is carbonyl or oxygen, especially carbonyl;

d) R₃ is hydroxy, O⁻M⁺, C₁-C₈alkylthio, C₃-C₈alkenylthio, C₃-C₈alkynylthio, benzylthio or phenylthio, especially hydroxy or a salt of formula O^{-M}⁺ wherein M⁺ is an agronomically acceptable metal cation or ammonium cation;

e) R₂ is chlorine, bromine, cyano, trifluoromethyl, difluoromethyl, difluorochloromethyl, difluoromethoxy, trifluoromethoxy, trifluoroethoxy, difluoromethylthio, trifluoromethylthio, trifluoroethylthio, methylthio, ethylthio, methylsulfonyl or ethylsulfonyl, especially trifluoromethyl;

or f) R₁ is a group —NR₆R₇ or a group —X₂—R₈ wherein X₂ is —O— or —NR₄₂—, or a group —X₃-L₁-R₉ wherein X₃ is —O— or —NR₅₇—.

Of those preferred compounds of formula I, special preference is given to the following compounds wherein

g) R₁ is a group —X₃-L₁-R₉ wherein X₃ is oxygen;

h) L₁ is a methylene or ethylene chain which may be substituted by methyl, ethyl, methoxy or by ethoxy, special emphasis being given to those compounds wherein R₁ as —X₃-L₁-R₉ is the side chain —O-L₁-O—R₇₀ wherein R₇₀ is C₁-C₃alkyl, allyl, propargyl, C₁-C₂alkoxy-C₁-C₂alkyl or C(O)—R₇₈ and R₇₈ is NR₁₂₇R₁₂₈;

i) R₁ as —X₃-L₁-R₉ is the group —O-L₁-X₂₀—R₇₀ wherein X₂₀ is especially oxygen and a carbon atom of the chain L₁ together with R₇₀ forms a C₂-C₆alkylene chain which may be interrupted once by oxygen and may be substituted once or twice by methyl;

j) R₁ is —X₃-L₁-X₂₀—R₇₀ wherein R₇₀ is C(O)NR₁₂₇R₁₂₈;

k) R₁ is the group —O-L₁-N(R₇₁)C(O)R₇₈ wherein R₇₁ is especially hydrogen and R₇₈ is especially C₁-C₄alkyl, cyclopropyl, phenyl, C₁-C₄alkoxy, methylamino or dimethylamino.

In a further especially preferred group of compounds that are preferred under the provisions of a) to f):

l) R₁ is a group —NR₆R₇ or a group —X₃-L₁-R₉ wherein X₃ is —NR₅₇— and wherein L₁ is a methylene or ethylene chain which may be substituted by methyl, ethyl, methoxy or by ethoxy, and of that group of compounds of formula I special mention should be made of those wherein —NR₆R₇ is a heterocyclic group selected from morpholin-4-yl, thiomorpholinyl-4-yl, pyrazol-1-yl and 1,2,4-triazol-1-yl, and wherein those groups may be substituted by methyl, trifluoromethyl, methoxy or by ethoxy;

m) R₁ is a group —NR₆R₇ wherein R₇ is C(X₇)R₃₀; and preferably R₆ is methyl or ethyl; X₇ is oxygen; and R₃₀ is C₁-C₆alkyl, C₃-C₆cycloalkyl or phenyl.

Also preferred are compounds of formula I wherein at least one of the linking members X₃ or X₂₀ is oxygen; preferably both are oxygen.

L₁ is preferably an unsubstituted C₁-C₃alkylene chain or a C₂alkylene chain substituted once by methyl.

In an outstanding group of compounds that are preferred under the provisions of a) to e):

o) R₁ is a group -L₁₁-X₁—R₅ wherein L₁₁ is C₁-C₂alkylene which may be substituted by methyl, ethyl, methoxy or by ethoxy, especially unsubstituted methylene;

p) R₁ is a group -L₁₁-X₁—R₅ wherein X₁ is oxygen, —C(O)—, —C(═NR₁₄ₐ)—, —C(O)O—, —C(O)NR_{14b}—, thio, sulfonyl, —NR₁₃SO₂—, —N(SO₂R_{14c})— or —NR₁₄— wherein R₁₄ is C₁-C₆alkoxycarbonyl or C₁-C₆alkylcarbonyl, X₁ being especially oxygen or —N(SO₂R_{14c})—;

q) R₁ is a group -L₁₁-X₁—R₅ wherein R₅ is C₁-C₆alkyl which may be substituted by halogen, cyano, C₁-C₄alkoxy, C₁-C₄alkoxycarbonyl, C₂-C₄alkenyl, C₂-C₄haloalkenyl, C₂-C₄alkynyl, C₃-C₆cycloalkyl, C₃-C₄alkenyloxy, C₃-C₄alkynyloxy, C₁-C₄haloalkoxy, C₃-C₄haloalkenyloxy, cyano-C₁-C₃alkoxy or by C₁-C₃alkoxy-C₁-C₃alkoxy; R₅ being especially C₁-C₄alkyl, C₃-C₄-alkenyl or C₃-C₄alkynyl, or C₁-C₃alkyl which is substituted from one to three times by fluorine, once or twice by chlorine, once or twice by methoxy or by ethoxy, once by cyano, allyloxy, propargyloxy, difluoromethoxy, trifluoromethoxy, methoxyethoxy or by C₃-C₆cycloalkyl;

r) R₁ is a group -L₁₁-X₁—R₅ wherein R₅ is phenyl or a three- to six-membered monocyclic ring system which may be aromatic, saturated or partially saturated and contains from 1 to 4 hetero atoms selected from nitrogen, oxygen and sulfur, wherein phenyl or the ring system is bound to the substituent X₁ directly or via a C₁-C₂alkylene group, and each ring system contains not more than 2 oxygen atoms and not more than two sulfur atoms, and wherein the ring system itself may be substituted from one to four times by C₁-C₃alkyl or by halogen and/or once by C₁-C₃alkoxy, C₁-C₃haloalkoxy, allyloxy, propargyloxy, C₁-C₃alkylthio or by C₁-C₃alkylsulfonyl; R₅ being especially phenyl or a three- to six-membered monocyclic, saturated ring system which contains 1 or 2 oxygen atoms, and wherein phenyl or the ring system is bound to the substituent X₁ either directly or via a C₁-C₂alkylene group, and wherein preferably the ring system itself is unsubstituted or may be substituted from one to four times by C₁-C₃alkyl and/or once by methoxy or by ethoxy.

In a very especially preferred group of compounds of formula I wherein R₁ is a group -L₁₁-X₁—R₅

s) the bidentate linking member -L₁₁-X₁— is preferably —CH₂O—, —CH₂CH₂O— or —CH₂N(SO₂CH₃)—. Compounds of formula I wherein -L₁₁-X₁—R₅ is CH₂OCH₂CH₂OCH₃, CH₂OCH₂CH₂OCH₂CH₃, CH₂OCH₂CF₃, CH₂OCH₂CH═CH₂, CH₂OCH₂C≡CH, CH₂OCH₂C≡CCH₃, CH₂OCH₂CH₂C═CH, CH₂OCH₂C≡N, CH₂OCH₂CH₂C≡N, CH₂OCH₂CH₂CH₂OCH₃, CH₂OCH₂CH₂OCH₂CH₂OCH₃, CH₂OCH₂CH₂OCF₃, CH₂OCH₂CH₂CH₂OCF₃, CH₂CH₂OCH₂CH₂OCH₃, CH₂N(SO₂CH₃)CH₃, CH₂N(SO₂CH₃)CH₂CH₃, CH₂N(SO₂CH₃)CH₂CF₃ or CH₂N(SO₂CH₃)CH₂CH₂OCH₃ are of very special interest.

In a further outstanding group of compounds that are preferred under the provisions of a) to e):

t) R₁ is a group -L₁₀-R₄ wherein L₁₀ is a direct bond or is a C₁-C₃alkylene or C₂-C₃alkenylene group which may be substituted from one to three times by halogen, methyl, ethyl, methoxy or by ethoxy, L₁₀ being especially a direct bond or an unsubstituted C₁-C₂alkylene group;

u) R₁ is a group -L₁₀-R₄ wherein R₄ is hydrogen, halogen, cyano or trifluoromethyl or R₄ is a three- to six-membered monocyclic ring system which may be saturated, partially saturated or aromatic and may contain 1 or 2 hetero atoms selected from nitrogen, oxygen and sulfur and may itself be substituted by methyl or methoxy or by methoxymethyl. R₁ as a group -L₁₀-R₄ is especially fluorine, chlorine, bromine, cyano, methyl, ethyl, n-propyl, isopropyl, cyclopropyl, trifluoromethyl, chloromethyl, bromomethyl, methoxymethyl, ethoxymethyl, dimethoxymethyl, diethoxymethyl, 1-(dimethoxy)ethyl, 1-(diethoxy)ethyl, 1-ethoxyethylene, tetrahydrofuran-2-yl, tetrahydrofuran-3-yl, 1,3-dioxolan-2-yl, 1,3-dioxolan-4-yl, (2-methyl-[1,3]dioxolan-2-yl), tetrahydropyran-2-yl, 4,5-dihydro-isoxazol-5-yl, 4,5-dihydro-isoxazol-5-yl, (3-methyl-4,5-dihydro-isoxazol-5-yl), phenyl, or phenyl substituted by halogen, methyl, trifluoromethyl, methoxy, trifluoromethoxy or by cyano.

Further groups of compounds of formula I that should be given special mention are those wherein X₁, X₂ and X₃ are sulfur, sulfinyl or sulfonyl.

In addition, very special mention should be made of a group of compounds wherein

R₁ is -L₁₁-X₁—R₅, —NR₆R₇, —X₂—R₈, —X₃-L₁-R₉, C₁-C₆haloalkyl, C₂-C₆haloalkenyl, C₂-C₆-haloalkynyl or halogen;

L₂, L₄, L₆ and 8 are each independently of the others C₁-C₄alkylene which may be substituted once, twice or three times by C₁-C₄alkyl, halogen or by C₁-C₄alkoxy and to which C₁-C₄alkylene group there may additionally be spirocyclically bound a C₂-C₅alkylene group, and wherein that C₂-C₅alkylene group may in turn be interrupted once or twice by oxygen, sulfur, sulfinyl or by sulfonyl and/or substituted by C₁-C₄alkyl or by C₁-C₄alkoxy;

L₃, L₅, L₇ and L₉ are each independently of the others C₁-C₄alkylene which may be substituted once, twice or three times by C₁-C₄alkyl, halogen or by C₁-C₄alkoxy;

R₂ is halogen, C₁-C₄haloalkyl, cyano, C₁-C₃haloalkoxy, C₁-C₄alkylthio, C₁-C₄alkylsulfinyl, C₁-C₄alkylsulfonyl, C₁-C₄haloalkylthio, C₁-C₄haloalkylsulfinyl or C₁-C₄haloalkylsulfonyl;

L₁₁ is a C₁-C₆alkylene, C₂-C₆alkenylene or C₂-C₆alkynylene group which may be substituted once, twice or three times by halogen, hydroxy, C₁-C₆alkoxy, C₃-C₆cycloalkyloxy, C₁-C₆-alkoxy-C₁-C₆alkoxy, C₁-C₆alkoxy-C₁-C₆alkoxy-C₁-C₆alkoxy or by C₁-C₂alkylsulfonyloxy;

X₁ is oxygen, —OC(O)—, —C(O)—, —C(═NR₁₄ₐ)—, —C(O)O—, —C(O)NR_{14b}—, —OC(O)O—, —N(R₁₀)—O—, —O—NR₁₁—, thio, sulfinyl, sulfonyl, —SO₂NR₁₂—, —NR₁₃SO₂—, —N(SO₂R_{14c})—, —N(R_{14d})C(O)— or —NR₁₄—;

R₁₀, R₁₁, R₁₂, R₁₃, R_{14b}, R_{14d} and R₁₄ are each independently of the others hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;

R₁₄ₐ is hydroxy, C₁-C₆alkoxy, C₃-C₆alkenyloxy, C₃-C₆alkynyloxy or benzyloxy;

R_{14c} is C₁-C₆alkyl;

R₅ is hydrogen or C₁-C₈alkyl, or is a C₁-C₈alkyl, C₃-C₈alkenyl or C₃-C₈alkynyl or C₃-C₆cycloalkyl group which may be substituted once, twice or three times by halogen, hydroxy, amino, formyl, nitro, cyano, mercapto, carbamoyl, C₁-C₆alkoxy, C₂-C₆alkenyl, C₂-C₆haloalkenyl, C₂-C₆alkynyl, C₂-C₆haloalkynyl, C₃-C₆cycloalkyl, halo-substituted C₃-C₆cycloalkyl, C₃-C₅-alkenyloxy, C₃-C₆alkynyloxy, C₁-C₆haloalkoxy, C₃-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆-alkoxy, C₁-C₆alkoxycarbonyl, C₁-C₆alkylcarbonyl, C₁-C₆alkylthio, C₁-C₆alkylsulfinyl, C₁-C₆-alkylsulfonyl, C₁-C₆haloalkylthio, C₁-C₆haloalkylsulfinyl, C₁-C₆haloalkylsulfonyl, benzyloxy, benzylthio, benzylsulfinyl, benzylsulfonyl, C₁-C₆alkylamino, di(C₁-C₆alkyl)amino, R₁₉R₂₀C═NO—, R₁₅S(O)₂O—, R₁₆N(R₁₇)SO₂—, rhodano, phenyl, phenoxy, phenylthio, phenylsulfinyl or by phenylsulfonyl; wherein the phenyl- or benzyl-containing groups may in turn be substituted by one or more C₁-C₆alkyl, C₁-C₆haloalkyl, C₁-C₆alkoxy, C₁-C₆haloalkoxy, halogen, cyano, hydroxy or nitro groups;

R₁₅, R₁₆, R₁₇, R₁₉ and R₂₀ are each independently of the others hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl, or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;

or R₅ is a three- to ten-membered monocyclic or fused bicyclic ring system which may be aromatic, saturated or partially saturated and may contain from 1 to 4 hetero atoms selected from nitrogen, oxygen and sulfur, and wherein the ring system is bound to the substituent X₁ directly or via a C₁-C₄alkylene, C₂-C₄alkenylene, C₂-C₄alkynylene, —N(R₁₈)—C₁-C₄alkylene, —S(O)—C₁-C₄alkylene or —SO₂—C₁-C₄alkylene chain, wherein each ring system may not be interrupted by —C(═O)—, —C(═S)—, —C(═NR₅₂)—, —N(═O)—, —S(═O)— or by —SO₂—, and each ring system may contain not more than 2 oxygen atoms and not more than two sulfur atoms, and the ring system itself may be substituted once, twice or three times by C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, hydroxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅-acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di(C₁-C₂alkyl)aminosulfonyl, di(C₁-C₄alkyl)amino, halogen, cyano, nitro, phenyl or by benzylthio, wherein phenyl and benzylthio may in turn be substituted on the phenyl ring by C₁-C₃alkyl, C₁-C₃haloalkyl, C₁-C₃alkoxy, C₁-C₃haloalkoxy, halogen, cyano or by nitro, and wherein the substituents on the nitrogen in the heterocyclic ring are other than halogen;

R₅ₐ is C₁-C₆alkyl, hydroxy, C₁-C₆alkoxy, cyano or nitro;

R₁₈ is hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl, or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;

R₆ is hydrogen, C₁-C₆alkyl, C₃-C₆alkenyl, C₃-C₆alkynyl, C₁-C₆haloalkyl, hydroxy, C₁-C₆alkoxy, —C(O)R₁₉ₐ or —C(S)R₂₀ₐ;

R₁₉ₐ and R₂₀ₐ are each independently of the other hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₂, R₂₂;

R₂₁ and R₂₂ are each independently of the other hydrogen, C₁-C₆alkyl, C₃-C₆alkenyl, C₃-C₆alkynyl or phenyl, and wherein phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃-alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₂₁ together with R₂₂ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₆ is -L₂-X₄—R₂₄; wherein

X₄ is oxygen, —NR₂₃—, —S—, —S(O)— or —S(O)₂—;

R₂₃ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₂₄ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₅)NR₂₅R₂₆, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₂₄ is C(O)—R₇₄ or C(S)—R₇₅;

X₅ is oxygen or sulfur;

R₂₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₂₆ is hydrogen, C₁-C₆alkyl, C₃-C₆alkenyl or C₃-C₆alkynyl;

or R₂₅ together with R₂₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₆ is -L₃-R₂₇;

R₂₇ is formyl, C₁-C₆alkylcarbonyl, C₃-C₆cycloalkylcarbonyl, benzoyl, C₁-C₆alkoxycarbonyl, cyano, C(X₆)NR₂₈R₂₉, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;

or R₂₇ is C₃-C₆cycloalkyl or C₅-C₆cycloalkenyl each of which may in turn be substituted once, twice or three times by C₁-C₄alkyl, halogen or by C₁-C₄alkoxy;

X₆ is oxygen or sulfur;

R₂₈ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₂₉ is hydrogen, C₁-C₆alkyl, C₃-C₆alkenyl or C₃-C₆alkynyl;

or R₂₈ together with R₂₉ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₇ is hydrogen, C₁-C₆alkyl, C₃-C₆alkenyl, C₃-C₆alkynyl, C₁-C₆haloalkyl, C₃-C₆cycloalkyl, phenyl, heteroaryl, C(X₇)R₃₀ or NR₃₃R₃₄;

X₇ is oxygen or sulfur;

R₃₀ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₃₁R₃₂;

R₃₁ and R₃₃ are each independently of the other hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₃₂ and R₃₄ are each independently of the other hydrogen, C₁-C₆alkyl, C₃-C₆alkenyl or C₃-C₆alkynyl;

or R₃₁ together with R₃₂ or R₃₃ together with R₃₄, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₇ is -L₄-X₈—R₃₅; wherein

X₈ is oxygen, —NR₃₆—, —S—, —S(O)— or —S(O)₂—;

R₃₆ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₃₅ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₉)NR₃₇R₃₈, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

X₉ is oxygen or sulfur;

R₃₇ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₃₈ is hydrogen, C₁-C₆alkyl, C₃-C₆alkenyl or C₃-C₆alkynyl;

or R₃₇ together with R₃₈ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₇ is -L₅-R₃₉;

R₃₉ is formyl, C₁-C₆alkylcarbonyl, C₃-C₆cycloalkylcarbonyl, benzoyl, C₁-C₆alkoxycarbonyl, cyano, C(X₁₀)NR₄₀R₄₁, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;

or R₃₉ is C₃-C₆cycloalkyl or C₅-C₆cycloalkenyl each of which may in turn be substituted once, twice or three times by C₁-C₄alkyl, halogen or by C₁-C₄alkoxy;

X₁₀ is oxygen or sulfur;

R₄₀ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₄₁ is hydrogen, C₁-C₆alkyl, C₃-C₆alkenyl or C₃-C₆alkynyl;

or R₄₀ together with R₄, and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₆ and R₇ together with the nitrogen atom to which they are bonded form a carbocyclic 3- to 7-membered, saturated or partially saturated or unsaturated monocyclic or bicyclic ring system which may be interrupted once by oxygen, once by sulfur, from one to three times by nitrogen and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃-haloalkylthio, cyano, nitro or by C₁-C₄alkoxycarbonyl; wherein each ring system may not be interrupted by —C(═O)—, —C(═S)—, —C(═NR₅ₐ)—, —N(═O)—, —S(═O)— or by —SO₂—;

R₅ₐ is C₁-C₆alkyl, hydroxy, C₁-C₆alkoxy, cyano or nitro;

X₂ is oxygen, —NR₄₂—, sulfur, —S(O)— or —S(O)₂—;

R₄₂ is hydrogen, C₁-C₆alkyl, C₃-C₆alkenyl, C₃-C₆alkynyl, C₁-C₆haloalkyl, C₃-C₆cycloalkyl, phenyl, heteroaryl, C(X₁₁)R₄₃ or NR₄₆R₄₇;

X₁₁ is oxygen or sulfur;

R₄₃ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₄₄R₄₅;

R₄₄ and R₄₅ are each independently of the other hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₄₅ and R₄₇ are each independently of the other hydrogen, C₁-C₆alkyl, C₃-C₆alkenyl or C₃-C₆alkynyl;

or R₄₄ together with R₄₅ or R₄₆ together with R₄₇, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₄₂ is -L₆-X₁₂—R₄₈; wherein

X₁₂ is oxygen, —NR₄₉—, —S—, —S(O)— or —S(O)₂;

R₄₉ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₄₈ is a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆alkylsulfonyl, cyano, C(X₁₃)NR₅₀R₅₁, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

X₁₃ is oxygen or sulfur;

R₅₀ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₅₁ is hydrogen, C₁-C₆alkyl, C₃-C₆alkenyl or C₃-C₆alkynyl;

or R₅₀ together with R₅, and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₄₂ is -L₇-R₅₂;

R₅₂ is formyl, C₁-C₆alkylcarbonyl, C₃-C₆cycloalkylcarbonyl, benzoyl, C₁-C₆alkoxycarbonyl, cyano, C(X₁₄)NR₅₃R₅₄, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl; or R₅₂ is C₃-C₆cycloalkyl or C₅-C₆cycloalkenyl each of which may in turn be substituted once, twice or three times by C₁-C₄alkyl, halogen or by C₁-C₄alkoxy;

X₁₄ is oxygen or sulfur;

R₅₃ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₅₄ is hydrogen, C₁-C₆alkyl, C₃-C₆alkenyl or C₃-C₆alkynyl;

or R₅₃ together with R₅₄ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₈ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₁₅)NR₅₅R₅₆, C₃-C₆cycloalkyl, phenyl, phenoxy or by 5- or 6-membered heteroaryl or heteroaryloxy, and wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

X₁₅ is oxygen or sulfur;

R₅₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₅₆ is hydrogen, C₁-C₆alkyl, C₃-C₆alkenyl or C₃-C₆alkynyl;

or R₅₅ together with R₅₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₈ is C(O)—R₇₆ or C(S)—R₇₇;

X₃ is oxygen, —NR₅₇—, sulfur, —S(O)— or —S(O)₂—;

R₅₇ is hydrogen, C₁-C₆alkyl, C₃-C₆alkenyl, C₃-C₆alkynyl, C₁-C₆haloalkyl, C₃-C₆cycloalkyl, phenyl, heteroaryl, C(X₁₆)R₅₈ or NR₆₁R₆₂;

X₁₆ is oxygen or sulfur;

R₅₈ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₅₉R₆₀;

R₅₉ and R₆₁ are each independently of the other hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₆₀ and R₆₂ are each independently of the other hydrogen, C₁-C₆alkyl, C₃-C₆alkenyl or C₃-C₆alkynyl;

or R₅₉ together with R₆₀ or R₆₁ together with R₆₂, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₅₇ is -L₈-X₁₇—R₆₃; wherein

X₁₇ is oxygen, —NR₆₄—, —S—, —S(O)— or —S(O)₂—;

R₆₄ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₆₃ is a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆alkylsulfonyl, cyano, C(X₁₈)NR₆₅R₆₆, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

X₁₈ is oxygen or sulfur;

R₆₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₆₆ is hydrogen, C₁-C₆alkyl, C₃-C₆alkenyl or C₃-C₆alkynyl;

or R₆₅ together with R₆₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₅₇ is -L₉-R₆₇;

R₆₇ is formyl, C₁-C₆alkylcarbonyl, C₃-C₆cycloalkylcarbonyl, benzoyl, C₁-C₆alkoxycarbonyl, cyano, C(X₁₉)NR₆₈R₆₉, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;

or R₆₇ is C₃-C₆cycloalkyl or C₅-C₆cycloalkenyl each of which may in turn be substituted once, twice or three times by C₁-C₄alkyl, halogen or by C₁-C₄alkoxy;

X₁₉ is oxygen or sulfur;

R₆₈ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₆₉ is hydrogen, C₁-C₆alkyl, C₃-C₆alkenyl or C₃-C₆alkynyl;

or R₆₈ together with R₆₉ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

L₁ is C₁-C₄alkylene which may be substituted once, twice or three times by C₁-C₄alkyl, halogen or by C₁-C₄alkoxy and to which C₁-C₄alkylene group there may be spirocyclically bound a further C₂-C₅alkylene group which may in turn be interrupted once or twice by oxygen, sulfur, sulfinyl or by sulfonyl and may be substituted by C₁-C₄alkyl or by C₁-C₄alkoxy;

or L₁ is C₁-C₄alkylene which may be substituted once, twice or three times by C₁-C₄alkyl, halogen or by C₁-C₄alkoxy, wherein a carbon atom of that C₁-C₄alkylene chain together with R₉ or with R₇₀ forms a further C₂-C₆alkylene chain which may be interrupted once or twice by oxygen, sulfur, sulfinyl or by sulfonyl and may be substituted by C₁-C₄alkyl or by C₁-C₄alkoxy;

R₉ is a group —X₂₀—R₇₀, wherein

X₂₀ is oxygen, —NR₇₁—, —S—, —S(O)— or —S(O)₂—;

R₇₁ is a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆alkylsulfonyl, cyano, C(X₂₁)NR₇₂R₇₃, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

X₂₁ is oxygen or sulfur;

R₇₂ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₇₃ is hydrogen, C₁-C₆alkyl, C₃-C₆alkenyl or C₃-C₆alkynyl;

or R₇₂ together with R₇₃ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₇₀ is hydrogen, C₁-C₆alkyl, C₃-C₆alkenyl, C₃-C₆alkynyl, C(O)—R₇₈, C(S)—R₇₉ or phenyl which may be substituted once, twice or three-times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

or R₉ is formyl, C₁-C₆alkylcarbonyl, C₃-C₆cycloalkylcarbonyl, benzoyl, C₁-C₆alkoxycarbonyl, cyano, C(X₃₅)NR₁₂₅R₁₂₆, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;

or R₉ is C₃-C₆cycloalkyl or C₅-C₆cycloalkenyl each of which may in turn be substituted once, twice or three times by C₁-C₄alkyl, halogen or by C₁-C₄alkoxy;

X₃₅ is oxygen or sulfur;

R₁₂₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₁₂₆ is hydrogen, C₁-C₆alkyl, C₃-C₆alkenyl or C₃-C₆alkynyl;

or R₁₂₅ together with R₁₂₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₇₄, R₇₅, R₇₆, R₇₇, R₇₈ and R₇₉ are each independently of the others hydrogen, C₁-C₆alkyl, C₃-C₆cycloalkyl, phenyl, heteroaryl, C₁-C₆alkoxy, C₃-C₆alkenyloxy, C₁-C₄alkylthio or NR₁₂₇R₁₂₈;

R₁₂₇ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₁₂₈ is hydrogen, C₁-C₆alkyl, C₃-C₆alkenyl or C₃-C₆alkynyl;

or R₁₂₇ together with R₁₂₈ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;

R₃ is hydroxy, O⁻M⁺ wherein M⁺ is a metal cation or an ammonium cation, or is halogen or S(O)ₚR₈₀ wherein R₈₀ is C₁-C₁₂alkyl, C₂-C₁₂alkenyl, C₂-C₁₂alkynyl, C₃-C₂allenyl, C₃-C₁₂-cycloalkyl or C₅-C₁₂cycloalkenyl; and p is 0, 1 or 2;

or R₈₀ is R₁₂₁—C₁-C₁₂alkylene or R₁₂₂—C₂-C₁₂alkenylene, wherein the alkylene or alkenylene chain may be interrupted by O—, —S—, —S(O)—, —SO₂— or by —C(O)— and/or substituted from one to five times by R₁₂₃; or R₈₀ is phenyl which may be substituted once, twice, three times, four times or five times by R₁₂₄;

R₁₂, and R₁₂₂ are each independently of the other halogen, cyano, rhodano, hydroxy, C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, C₂-C₆alkenylthio, C₂-C₆alkynylthio, C₁-C₆alkylsulfonyloxy, phenylsulfonyloxy, C₁-C₆alkylcarbonyloxy, benzoyloxy, C₁-C₄alkoxycarbonyloxy, C₁-C₆alkylcarbonyl, C₁-C₄-alkoxycarbonyl, benzoyl, aminocarbonyl, C₁-C₄alkylaminocarbonyl, C₃-C₆cycloalkyl, phenyl, phenoxy, phenylthio, phenylsulfinyl or phenylsulfonyl, wherein the phenyl-containing groups may in turn be substituted once, twice or three times by halogen, C₁-C₃alkyl, C₁-C₃haloalkyl, hydroxy, C₁-C₃alkoxy, C₁-C₃haloalkoxy, cyano or by nitro;

R₁₂₃ is hydroxy, halogen, C₁-C₆alkyl, C₁-C₆alkoxy, C₁-C₆alkylthio, C₁-C₆alkylsulfinyl, C₁-C₆alkylsulfonyl, cyano, carbamoyl, carboxy, C₁-C₄alkoxycarbonyl or phenyl, wherein phenyl may be substituted once, twice or three times by hydrogen, C₁-C₄alkyl, C₁-C₄haloalkyl, C₃-C₄alkenyl, C₃-C₄alkynyl or by C₁-C₄alkoxy;

R₂₄ is halogen, C₁-C₃alkyl, C₁-C₃haloalkyl, hydroxy, C₁-C₃alkoxy, C₁-C₃haloalkoxy, cyano or nitro;

A₁ is —C(R₁₁₂R₁₁₃)— or —NR₁₁₄—;

A₂ is —C(R₁₁₅R₁₁₆)ₘ—, —C(═O)—, —O—, —NR₁₁₇— or —S(O)_{q}—;

A₃ is —C(R₁₁₈R₁₁₉)— or —NR₁₂₀—;

with the proviso that A₂ is other than —O— or —S(O)_{q}— when A₁ is —NR₁₁₄— and/or A₃ is —NR₁₂₀;

R₁₁₂ and R₁₁₈ are each independently of the other hydrogen, C₁-C₄alkyl, C₂-C₄alkenyl, C₂-C₄alkynyl, C₁-C₄alkylthio, C₁-C₄alkylsulfinyl, C₁-C₄alkylsulfonyl, C₁-C₄alkoxycarbonyl, hydroxy, C₁-C₄alkoxy, C₃-C₄alkenyloxy, C₃-C₄alkynyloxy, hydroxy-C₁-C₄alkyl, C₁-C₄alkylsulfonyloxy-C₁-C₄alkyl, halogen, cyano or nitro;

R₁₁₃ and R₁₁₉ are each independently of the other hydrogen, C₁-C₄alkyl or C₁-C₄alkylthio, C₁-C₄alkylsulfinyl or C₁-C₄alkylsulfonyl;

or R₁₁₃ together with R₁₁₂ and/or R₁₁₉ together with R₁₁₈ form a C₂-C₅alkylene chain which may be interrupted by —O—, —C(O)O— or by —S(O)ᵣ—;

R₁₁₄ and R₁₂₀ are each independently of the other hydrogen, C₁-C₄alkyl, C₁-C₄haloalkyl, C₃-C₄alkenyl, C₃-C₄alkynyl or C₁-C₄alkoxy;

R₁₁₅ is hydrogen, hydroxy, C₁-C₄alkyl, C₁-C₄haloalkyl, C₁-C₃hydroxyalkyl, C₁-C₄alkoxy-C₁-C₃alkyl, C₁-C₄alkylthio-C₁-C₃alkyl, C₁-C₄alkylcarbonyloxy-C₁-C₃alkyl, C₁-C₄alkylsulfonyloxy-C₁-C₃alkyl, tosyloxy-C₁-C₃alkyl, di(C₁-C₄alkoxy)C₁-C₃alkyl, C₁-C₄alkoxycarbonyl, formyl, C₃-C₅oxacycloalkyl, C₃-C₅thiacycloalkyl, C₃-C₄dioxacycloalkyl, C₃-C₄dithiacycloalkyl, C₃-C₄-oxathiacycloalkyl, C₁-C₄alkoxyiminomethyl, carbamoyl, C₁-C₄alkylaminocarbonyl or di(C₁-C₄alkyl)aminocarbonyl;

or R₁₅ together with R₁₁₂ or R₁₁₃ or R₁₁₄ or R₁₁₆ or R₁₁₈ or R₁₁₉ or R₁₂₀ or, when m is 2, also with a second R₁₁₅ form a C₁-C₄alkylene bridge;

R₁₁₆ is hydrogen, C₁-C₃alkyl or C₁-C₃haloalkyl;

R₁₁₇ is hydrogen, C₁-C₃alkyl, C₁-C₃haloalkyl, C₁-C₄alkoxycarbonyl, C₁-C₄alkylcarbonyl or di(C₁-C₄alkyl)aminocarbonyl;

m is 1 or 2; and

q and r are each independently of the other 0, 1 or 2;

and also Examples 1.009, 1.010, 1.033 to 1.045, 1.092, 1.097 to 1.110, 1.153 to 1.156, 1.158 to 1.169, 1.189, 1.192, 1.195 to 1.201, 2.009, 2.010, 2.033 to 2.045, 2.091, 2.096 to 2.109, 2.152 to 2.155, 2.157 to 2.168, 2.188, 2.191, 2.194 to 2.200, 3.009, 3.010, 3.033 to 3.045, 3.091, 3.096 to 3.109, 3.152 to 3.155, 3.157 to 3.168, 3.188, 3.191, 3.194 to 3.200.

The compounds of formula I can be prepared by means of processes that are known per se and are described, for example, in WO 00/15615, EP-A-0 316 491, EP-A-1 352 901 and WO 02/16305.

Compounds of formula I can be prepared, for example, by
a) converting a compound of formula IIa 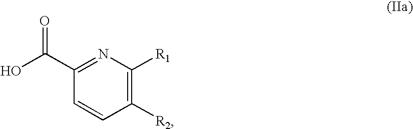
wherein R₁ and R₂ are as defined hereinbefore, into a compound of formula IIb 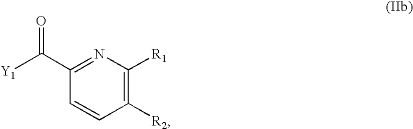
wherein Y₁ is a leaving group, e.g. halogen, cyano, acyloxy or phenoxy which may be substituted by an electron-withdrawing group, e.g. halogen, trifluoromethyl, nitro, cyano, C₁-C₄alkylcarbonyl, C₁-C₄alkoxycarbonyl or C₁-C₄alkylsulfonyl, or the like, and then reacting that compound in the presence of a base, e.g. triethylamine, Hünig's base, sodium hydrogen carbonate or potassium carbonate, with a dione of formula III 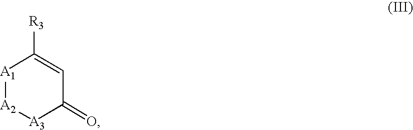
wherein A₁, A₂ and A₃ are as defined hereinbefore and R₃ is hydroxy or O⁻M⁺ wherein M⁺ is as defined hereinbefore, and then treating the reaction mixture in the presence of the base used, e.g. triethylamine, with the aid of a cyanide-containing catalyst, e.g. acetone cyanohydrin, trimethylsilyl cyanide, copper cyanide, sodium cyanide or potassium cyanide, or by means of fluoride ions, e.g. potassium fluoride, or by means of dimethylaminopyridine; or
b) converting a compound of formula XIIIa 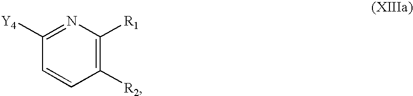
wherein R₁ and R₂ are as defined hereinbefore and Y₄ is halogen or trifluoromethanesulfonyloxy, under carbonylation conditions at elevated pressure and elevated temperature in the presence of a palladium catalyst having suitable ligands, e.g. PdCl₂(PPh₃)₂, Pd(PPh₃)₄, Pd₂(dba)₃, Pd(CH₃CN)₂(PPh₃)₂, Pd(OAc)₂ or (racBINAP)PdCl₂, and optionally in the presence of an auxiliary catalyst, e.g. triphenylphosphine, tri(tert-butyl)phosphine, (Ph₃)₂PCH₂CH₂P(Ph₃)₂ or (Ph₃)₂PCH₂CH₂CH₂P(Ph₃)₂, and in the presence of a base, e.g. triethylamine, and optionally further adjuvants, e.g. LiCl or Li₂CO₃, using carbon monoxide and a dione of formula III or using the tautomeric form of a dione of formula IIIa 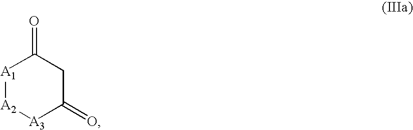
wherein A₁, A₂ and A₃ are as defined hereinbefore and R₃ is hydroxy, into a compound of formula XIa 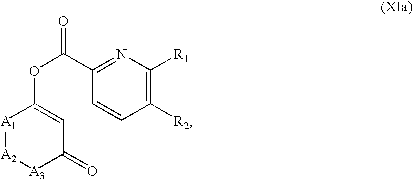
and/or its isomeric form XIb 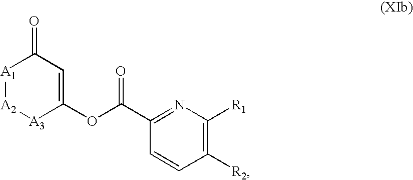
wherein A₁, A₂, A₃, R₁ and R₂ are as defined hereinbefore, and then treating that compound with the aid of a cyanide-containing catalyst, e.g. acetone cyanohydrin, trimethylsilyl cyanide, copper cyanide, sodium cyanide or potassium cyanide, in the presence of a trialkylamine base, e.g. triethylamine, to obtain a compound of formula I; or
c) when X₂ or X₃ is oxygen or sulfur, reacting a compound of formula Ia 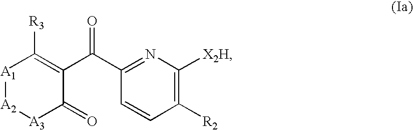
wherein A₁, A₂, A₃, R₂ and R₃ are as defined hereinbefore and X₂ is oxygen or sulfur, in the presence of a suitable base, e.g. potassium carbonate, anhydrous sodium hydroxide or sodium hydride, with an alkylating agent of formula IVa or IVb
Y₂—R₈ (IVa)
or
Y₂-L₁-R₉ (IVb),
wherein R₈ is an unsubstituted or substituted C₁-C₆alkyl, C₃-C₆alkenyl or C₃-C₆alkynyl group, and R₉ and L₁ are as defined hereinbefore, and Y₂ is a leaving group such as chlorine, bromine, iodine, mesyloxy or tosyloxy; or
d) when X₂ or X₃ is oxygen, reacting a compound of formula Ia 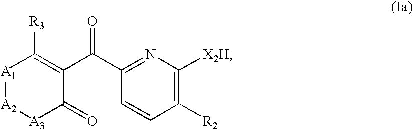
wherein A₁, A₂, A₃, R₂ and R₃ are as defined hereinbefore and X₂ is oxygen, in the presence of a bis-diaza-alkoxycarboxylate of formula ROC(O)—N═C═N—COOR or a bis-diazaalkyl-carbamoyl of formula RNHC(O)—N═C═N—C(O)NHR, wherein R is a C₁-C₆alkyl or C₅-C₆-cycloalkyl group, and a phosphine, e.g. triphenylphosphine or tri(tert-butyl)phosphine, with an alcohol of formula Va or Vb
HO—R₈ (Va)
or
HO-L₁-R₉ (Vb),
wherein R₈ is an unsubstituted or substituted C₁-C₆alkyl, C₃-C₆alkenyl or C₃-C₆alkynyl group, and R₉ and L₁ are as defined hereinbefore; or
e) reacting a compound of formula Ib 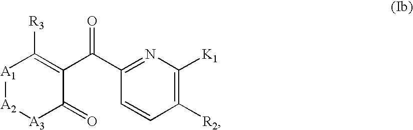
wherein A₁, A₂, A₃, R₂ and R₃ are as defined hereinbefore and K₁ is a leaving group, e.g. halogen or alkylsulfonyl, in the presence of a base, e.g. potassium tert-butanolate, sodium amylate, sodium hydride, dry sodium or potassium hydroxide, or an amine, e.g. triethylamine, Hünig's base or dimethylaminopyridine, with an alcohol or a mercaptan of formula Vc or Vd
HX₂—R₈ (Vc)
or
HX₃-L₁-R₉ (Vd),
wherein R₈ is an unsubstituted or substituted C₁-C₆alkyl, C₃-C₆alkenyl or C₃-C₆alkynyl group, and L₁ and R₉ are as defined hereinbefore, and X₂ or X₃ is oxygen or sulfur, or with an amine of formula Ve or VI
HNR₅₇-L₁-R₉ (Ve)
or
HNR₆R₇ (VI),
wherein L₁, R₆, R₇, R₉ and R₅₇ are as defined hereinbefore; or
f) reacting a compound of formula Ic 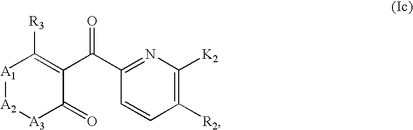
wherein A₁, A₂, A₃, R₂ and R₃ are as defined hereinbefore and K₂ is a group capable of appropriate functionalisation -L₁₀-K₂₀, -L₁₁-X₁—K₂₁, —X₂—K₂₂, —X₃-L₁-K₂₃ wherein L₁₀, L₁₁, L₁, X₁, X₂ and X₃ are as defined hereinbefore and K₂₀, K₂₁, K₂₂ and K₂₃ are a functionalisation group, e.g. hydroxy, chlorine, bromine, iodine, mesyloxy, tosyloxy, formyl or carbonyl, either with an appropriate alkylating agent of formula VII or VIIa
Y₃—R₉ (VII)
or
Y₃—X₂₀—R₇₀ (VIIa),
or with a ketalisation agent or a nucleophilic reagent of formula VIII
HX₂₀—R₇₀ (VII),
or its salt of formula VIIIa
M^{+ -}X₂₀—R₇₀ (VIIIa),
wherein R₉, R₇₀ and X₂₀ are as defined hereinbefore and Y₃ is a leaving group such as bromine, iodine, tosyloxy or C₁-C₄alkylsulfonyloxy, and M⁺ is a metal cation of an alkali metal base, such as lithium, sodium or potassium, optionally in the presence of an additional base, or, in the case of ketalisation of a carbonyl function, in the presence of an additional acid, e.g. p-toluenesulfonic acid, trifluoroacetic acid or sulfuric acid; or
g) when R₁ is -L₁₀-R₄ or -L₁₁-X₁—R₅ wherein L₁₀ or L₁₁ is especially an unsubstituted or substituted C₂-C₆alkenylene or a C₂-C₆alkynylene group and R₄, when L₁₀ is a direct bond, is especially a five- to ten-membered, monocyclic or fused bicyclic ring system which may be aromatic or partially saturated and which may contain from 1 to 4 hetero atoms selected from nitrogen, oxygen and sulfur, reacting a compound of formula Id 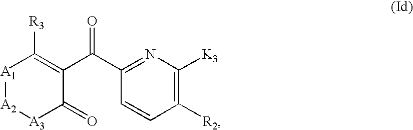
wherein A₁, A₂, A₃, R₂ and R₃ are as defined hereinbefore and K₃ is a group capable of functionalisation, such as chlorine, bromine, iodine or trifluoromethylsulfonyloxy, by means of a —C—C— linking Suzuki, Stille, Sonogashira or Heck reaction, in the presence of a noble metal catalyst having suitable ligands, e.g. PdCl₂(PPh₃)₂, Pd(PPh₃)₄, Pd₂(dba)₃, Pd(CH₃CN)₂(PPh₃)₂, Pd(OAc)₂, Rh, Cu, CuCl or CuI, and in the presence of a base, e.g. triethylamine, Hünig's base, sodium tert-butanolate, potassium tert-butanolate, sodium carbonate, potassium carbonate, caesium carbonate, potassium fluoride or caesium fluoride, and optionally further adjuvants, e.g. LiCl or Li₂CO₃, or an additional auxiliary catalyst, e.g. triphenylphosphine, tri(tert-butyl)phosphine, (Ph₃)₂PCH₂CH₂P(Ph₃)₂ or (Ph₃)₂PCH₂CH₂CH₂P(Ph₃)₂, with a boronic acid, e.g. of formula Xa or Xb
(HO)₂B-L₁₀-R₄ (Xa)
or
(HO)₂B-L₁₁-X₁—R₅ (Xb),
or with a tin compound of formula Xc or Xd
RₑR_{f}R_{g}Sn-L₁₀-R₄ (Xc)
or
RₑR_{f}R_{g}Sn-L₁₁-X₁—R₅ (Xd),
or with an ethynyl compound of formula Xe or Xf having a functionality in accordance with R₁
H—C≡C-L_{10b}-R₄ (Xe)
or
H—C≡C-L_{11b}-X₁—R₅ (Xf),
or with a vinyl compound of formula Xg or Xh having a functionality in accordance with R₁
H—CH═CH-L₁₀ₐ-R₄ (Xg)
or
H—CH═CH-L₁₁ₐ-X₁—R₅ (Xh),
wherein L₁₀, L₁₁, R₄, R₅ and X₁ are as defined hereinbefore, Rₑ, R_{f} and R_{g} are each independently of the others C₁-C₈alkyl and L₁₀ₐ, L_{10b}, L₁₁ₐ and L_{11b} are appropriate subgroups of the groups L₁₀ and L₁₁, such as especially a direct bond or a C₁-C₄alkylene group which may be substituted once, twice or three times by C₁-C₆alkyl, halogen, hydroxy, C₁-C₆alkoxy or by C₁-C₃alkoxy-C₁-C₃alkoxy; or
h) for the preparation of compounds of formula I wherein the substituent definitions include sulfinyl or sulfonyl groups, treating a compound of formula I wherein the corresponding substituent definitions include thio groups with an oxidising agent, e.g. peracetic acid, trifluoroperacetic acid, m-chloro-perbenzoic acid, hydrogen peroxide, sodium perbromate, sodium iodate, sodium hypochloride, chlorine or bromine.

In addition, compounds of formula I wherein R₃ is other than hydroxy or halogen can be prepared, in accordance with conversion methods generally known from the literature, by nucleophilic substitution reactions of chlorides of formula I wherein R₃ is chlorine, which can be obtained from compounds of formula I wherein R₃ is hydroxy, likewise in accordance with known methods, by reaction with a chlorinating agent, such as phosgene, thionyl chloride or oxalyl chloride. In that process, for example, mercaptans, thiophenols or heterocyclic thiols are reacted in the presence of a base, for example 5-ethyl-2-methylpyridine, diisopropylethylamine, triethylamine, sodium hydrogen carbonate, sodium acetate or potassium carbonate. Furthermore, compounds of formula I wherein the substituent R₃ is a mercapto group can be oxidised in analogy to known standard methods, for example using peracids, e.g. metachloroperbenzoic acid (m-CPBA) or peracetic acid, to form the corresponding sulfoxides and/or sulfones of formula I. In the process, the degree of oxidation at the sulfur atom (—S(O)— or —SO₂—) can be controlled by the amount of oxidising agent.

In the case of process a), as a result of reaction of a dione of formula III with an acid of formula IIa in the presence of a suitable coupling reagent, e.g. dicyclohexylcarbodiimide, N-ethyl-N′-(3-dimethylamino-propyl)-carbodiimide (EDC), 2-chloro-1-methyl-pyridinium iodide or N,N-dimethyl-(1-chloro-2-methyl-propen)amine, or as a result of reaction of a dione of formula III with an activated form of the acid, e.g. an acid chloride, of formula IIb, wherein Y₁ is chlorine, in the presence of a base, e.g. triethylamine, Hünig's base or potassium carbonate, there is obtained a corresponding enol ester compound of formula XIa and/or Xib 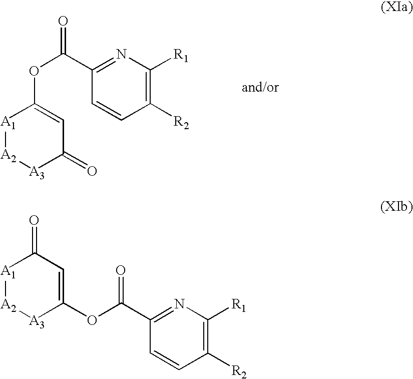
wherein A₁, A₂, A₃, R₁ and R₂ are as defined hereinbefore, which may then either be rearranged directly in situ by adding catalytic amounts of cyanide ions, e.g. from about 1% to about 15% acetone cyanohydrin, to form the compound of formula I, or may first be isolated and purified and then, in a second step, rearranged in the presence of catalytic amounts of from about 0.1% to about 5% potassium cyanide ions or from about 0.5% to about 10% acetone cyanohydrin, and a fresh amount of a trialkylamine base, e.g. from about 0.1 to about 3 equivalents of triethylamine, preferably from 1 to about 1.4 equivalents, to form the compound of formula I, as shown above using the exemplified formulae XIa and XIb to form compounds of formula I. This process may be shown in general terms in Scheme 1 using the example of the preparation of compounds of formula I.

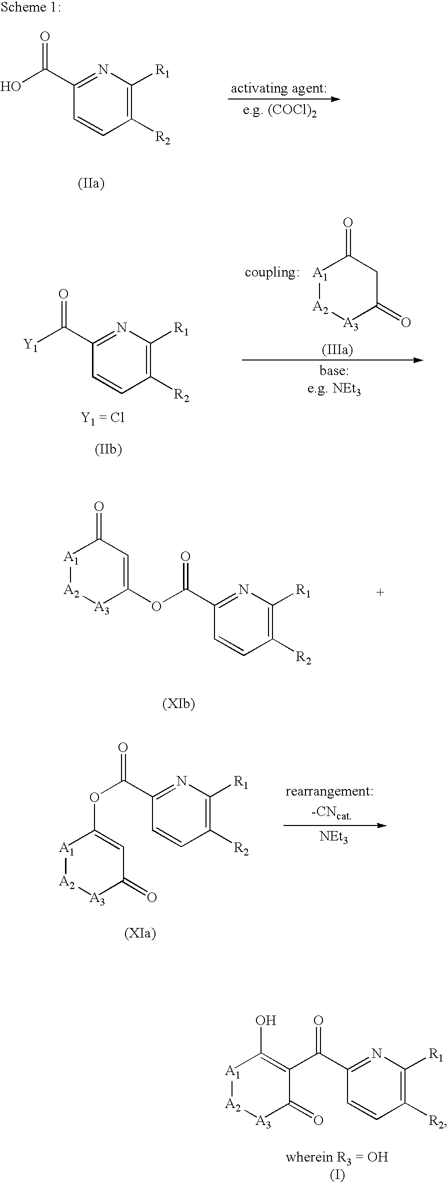

In a preferred process for the preparation of compounds of formula Ia 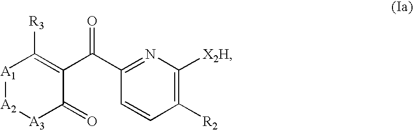
in which A₁, A₂, A₃, R₂ and R₃ are as defined hereinbefore and X₂ is oxygen or sulfur and which are used as starting materials in process variants c) and d), for example, either a compound of formula Ie 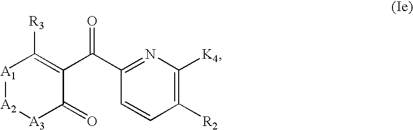
wherein A₁, A₂, A₃, R₂ and R₃ are as defined hereinbefore and K₄ is a cleavable group, such as methoxy when X₂ is oxygen, or is a disulfide bridge of a dimeric compound 1e, is reacted in the presence of an ether-cleaving reagent, e.g. boron trichloride, boron tribromide, aluminium chloride, sodium methylmercaptide, sodium ethylmercaptide or trimethylsilyl iodide, or a compound of formula Ie wherein K₄ is benzyloxy or dimeric disulfide is reduced catalytically in the presence of hydrogen.

The compounds of formula If used in process variants c), d), e), f) and g) 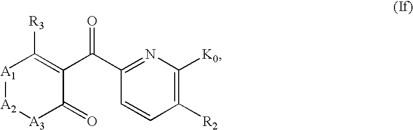
wherein A₁, A₂, A₃, R₂ and R₃ are as defined hereinbefore and K₀ is a sub-group of R₁ capable of functionalisation, as defined especially in accordance with the meanings of K₁, K₂, K₃ and/or K₄ above, such as especially fluorine, chlorine, bromine, iodine, hydroxy, methylthio, methylsulfonyl, bromomethyl, hydroxymethyl, formyl, methylcarbonyl, 2-hydroxyethoxy, 2-bromoethoxy, benzyloxy or dimeric disulfide, can likewise be prepared in accordance with process variant a) or in accordance with process variant b) from the corresponding compounds of formula XII or XIII 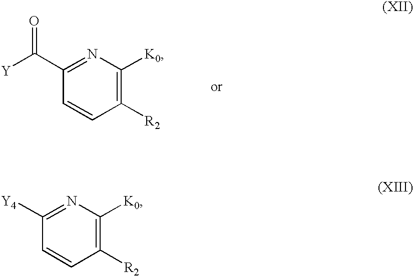
wherein K₀, R₂ and Y₄ are as defined hereinbefore; Y is C₁-C₄alkoxy, benzyloxy, hydroxy, fluorine, chlorine, cyano or phenoxy which may be substituted by an electron-withdrawing group, e.g. halogen, trifluoromethyl, nitro, cyano, C₁-C₄alkylcarbonyl, C₁-C₄alkoxycarbonyl or C₁-C₄alkylsulfonyl, Y being especially C₁-C₄alkoxy, benzyloxy, hydroxy, chlorine or cyano; and K₀ being especially hydroxy (corresponding to formula Ia wherein X₂ is oxygen), K₁ wherein K₁ is, for example, halogen or methylsulfonyl (corresponding to formula Ib), K₃ wherein K₃ is, for example, chlorine, bromine or iodine (corresponding to formula Id), K₄ wherein K₄ is, for example, methylthio, benzyloxy or methoxy (corresponding to formula Ie), or a disulfide bridge of a dimeric compound XII or XIII (corresponding to formula Ie wherein X₂ is sulfur),
aa) by reacting the compound in question with a dione of formula III 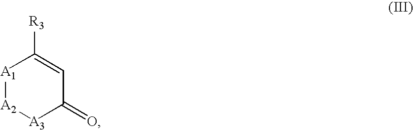
wherein R₃ is hydroxy or O⁻M⁺ and A₁, A₂, A₃ and M⁺ are as defined hereinbefore, or
bb) by reacting a hydroxy or mercapto compound of formula Ia 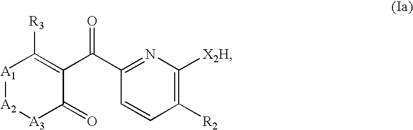
wherein A₁, A₂, A₃, R₂ and R₃ are as defined hereinbefore and X₂ is oxygen or sulfur, in accordance with process variant c) with an appropriate alkylating agent of formula IVc
Y₂-L₁-K₂₃ (IVc),
or
cc) in accordance with process variant d) with the appropriate alcohol of formula Vf
HO-L₁-K₂₃ (Vf),
or
dd) in accordance with process variant e) by treating a compound of formula Ib 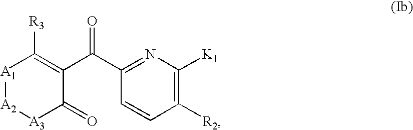
wherein A₁, A₂, A₃, R₂ and R₃ are as defined hereinbefore and K₁ is halogen or alkylsulfonyl, with an alcohol or mercaptan of formula Vg or Vh
HK₁ (Vg)
or
HX₃-L₁-K₂₃ (Vh),
or with an amine of formula VIc
HNR₆-L₁-K₂₃ (VIc),
wherein K₁ is methoxy or methylthio and X₃ is oxygen or sulfur, and K₂₃, L₁, R₆ and Y₂ are as defined hereinbefore.

The starting materials of formula IIa 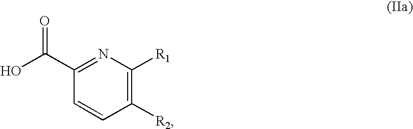
and of formula IIb 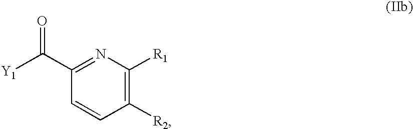
wherein R₁, R₂ and Y₁ are as defined hereinbefore, and also compounds of formula IId 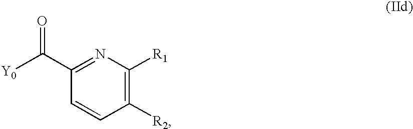
wherein Y₀ is C₁-C₄alkoxy, benzyloxy or phenoxy which may be unsubstituted or substituted by an electron-withdrawing group such as halogen, trifluoromethyl, nitro, cyano, C₁-C₄alkylcarbonyl, C₁-C₄alkoxycarbonyl or C₁-C₄alkylsulfonyl, which are used as starting materials in the preparation of compounds of formula IIa, can be prepared analogously to known methods, as described e.g. in EP-A-0 353 187, by converting a compound of formula XIII 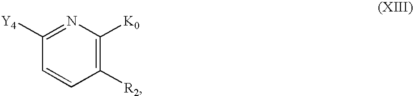
wherein R₂ is as defined hereinbefore, K₀ is hydrogen, methoxy, methylthio, methylsulfonyl, halogen or another group R₁ that is stable in this process, and Y₄ is chlorine, bromine or trifluoromethylsulfonyloxy, under carbonylation conditions at elevated pressure and elevated temperatures in the presence of a palladium catalyst having suitable ligands, e.g. PdCl₂(PPh₃)₂, Pd(PPh₃)₄, Pd(CH₃CN)₂(PPh₃)₂, Pd₂(dba)₃, Pd(OAc)₂ or (racBINAP)PdCl₂, and optionally in the presence of an auxiliary catalyst, e.g. triphenylphosphine, tri(tert-butyl)phosphine, (Ph₃)₂PCH₂CH₂P(Ph₃)₂ or (Ph₃)₂PCH₂CH₂CH₂P(Ph₃)₂, and in the presence of a base, e.g. triethylamine, using carbon monoxide and an alcohol of formula IX
R₀—OH (IX)
wherein R₀ is C₁-C₄alkyl, benzyl or phenyl which may be unsubstituted or substituted by an electron-withdrawing group such as halogen, trifluoromethyl, nitro, cyano, C₁-C₄alkylcarbonyl, C₁-C₄alkoxycarbonyl or C₁-C₄alkylsulfonyl, into a compound of formula XIIa 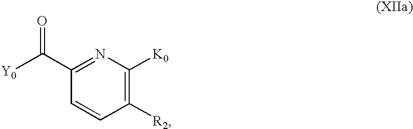
wherein K₀, R₂ and Y₀ are as defined hereinbefore, and then converting that compound in known conversion reactions, e.g. hydrolysis, addition and/or substitution reactions and subsequent hydrolysis, into the compound of formula XII 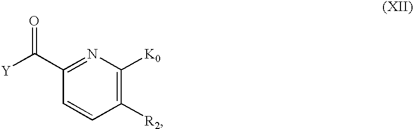
or of formula II 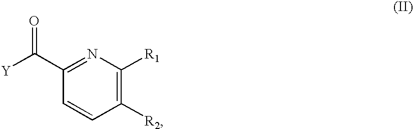
wherein K₀, R₁ and R₂ are as defined hereinbefore and Y is C₁-C₄alkoxy, benzyloxy, hydroxy, fluorine, chlorine, bromine, cyano or phenoxy which may be unsubstituted or substituted by an electron-withdrawing group, e.g. halogen, trifluoromethyl, nitro, cyano, C₁-C₄alkylcarbonyl, C₁-C₄alkoxycarbonyl or C₁-C₄alkylsulfonyl.

For example, a compound of formula XIIIb 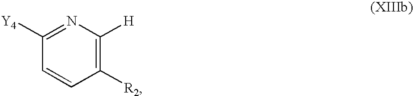
wherein R₂ and Y₄ are as defined hereinbefore, can be converted by carbonylation or by means of a Grignard reaction and CO₂ into a compound of formula XIIb 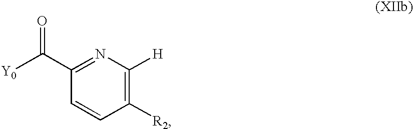
wherein R₂ and Y₀ are as defined hereinbefore, which is then converted in the presence of an oxidising agent, e.g. hydrogen peroxide or the hydrogen peroxide/urea adduct in the presence of trifluoroacetic anhydride, into an N-oxido compound of formula XV 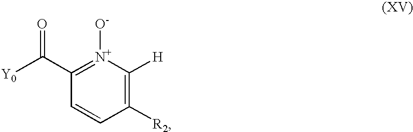
wherein R₂ and Y₀ are as defined hereinbefore, which is then either
a) reacted in the presence of phosphorus oxychloride or trifluoroacetic anhydride to form a hydroxy compound of formula XIIc 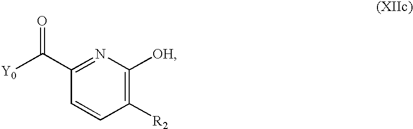
or
b) when a suitable nucleophile of formula Vi is used
HR₁ (Vi)
or when a nucleophile of formula VIb is used
HNR₆C(O)R₃₀ (VIb),
wherein R₁, R₆, R₃₀ are as defined hereinbefore, converted in the presence of an activating reagent, e.g. oxalyl chloride or trifluoroacetic anhydride, and optionally in the presence of an acid-binding agent, e.g. triethylamine or Hünig's base, directly into the compounds of formula IId 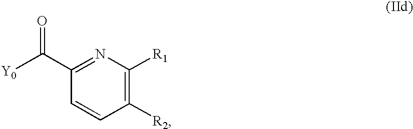
wherein R₁, R₂ and Y₀ are as defined hereinbefore and R₁ is especially a group —NR₄C(O)R₃₀, which can then be converted in accordance with a) for the isolation of an intermediate of formula XIIc by known and general conversion methods such as halogenation, e.g. by means of dichlorophenyl phosphate, further nucleophilic reactions with alcohols, mercaptans or amines of formula
HX₃-L₁-R₉ (V)
or
HNR₆R₇ (VI),
wherein X₃, L₁, R₆, R₇, R₉ are as defined hereinbefore, as described above under process conditions c) to h) into compounds of formula XII 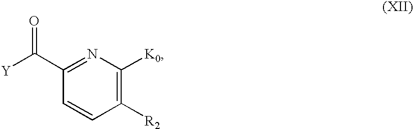
or of formula II 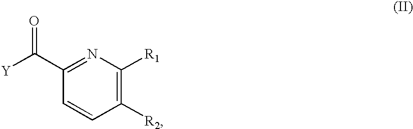
wherein K₀, R₁, R₂ and Y are as defined hereinbefore.

The starting materials of formula IId 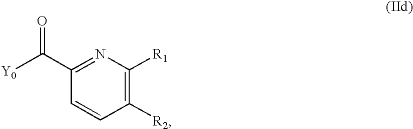
wherein R₁ is a group —X₂—R₈ or —X₃-L₁-R₉, X₂ and X₃ are oxygen, and L₁, R₂, R₉ and Y₀ are as defined hereinbefore, and R₈ is an unsubstituted or substituted C₁-C₆alkyl, C₃-C₆alkenyl or C₃-C₆alkynyl group, can also be prepared either by treating a compound of formula XIId 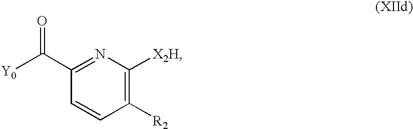
wherein R₂ and Y₀ are as defined hereinbefore, and X₂ is oxygen or sulfur, in accordance with process variant c) in the presence of a suitable base with an alkylating agent of formula
Y₂—R₈ (IVa)
or
Y₂-L₁-R₉ (IVb),
wherein L₁, R₉ and Y₂ are as defined hereinbefore and R₈ is an unsubstituted or substituted C₁-C₆alkyl, C₃-C₆alkenyl or C₃-C₆alkynyl group; or
by reacting a compound of formula XIId 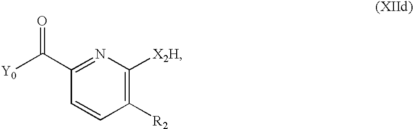
wherein R₂ and Y₀ are as defined hereinbefore and X₂ is oxygen, in accordance with process variant d) simultaneously in the presence of a bis-diaza-alkoxycarboxylate of formula ROC(O)—N═C═N—COOR or a bis-diaza-alkylcarbamoyl of formula RNHC(O)—N═C═N—C(O)NHR, wherein R is a C₁-C₆alkyl or C₅-C₆cycloalkyl group, and in the presence of a phosphine, e.g. triphenylphosphine or tri(tert-butyl)phosphine, with an alcohol of formula
HO—R₈ (Va)
or
HO-L₁-R₉ (Vb),
wherein R₈ is an unsubstituted or substituted C₁-C₆alkyl, C₃-C₆alkenyl or C₃-C₆alkynyl group, and R₉ and L₁ are as defined hereinbefore, respectively.

The reaction described in accordance with process variant d) is generally known as a Mitsunobu reaction and is especially suitable for the preparation of those compounds of formulae I and lid wherein R₁ is a group —X₂—R₈, —X₃-L₁-R₉ or —X₁-L₁-X₂₀—R₇₀ and X₂ and X₃ are oxygen and R₈ and L₁ are a C₁-C₄alkylene group branched in the alpha-position or substituted in that position by halogen or by alkoxy, or together with R₉ or with R₇₀ forms, by means of a further C₂-C₆alkylene chain, a 3- to 6-membered ring system.

Furthermore, the starting materials of formula IId 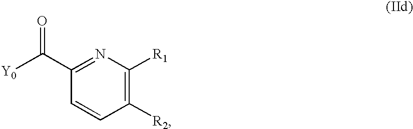
wherein R₁ is a group -L₁₀-R₄ or -L₁₁-X₁—R₅ and wherein L₁₀ and L₁₁ are especially an unsubstituted or substituted C₂-C₆alkenylene or a C₂-C₆alkynylene group and R₄, when L₁₀ is a direct bond, is especially a three- to ten-membered, monocyclic or fused bicyclic ring system which may be aromatic or partially saturated, and which may contain from 1 to 4 hetero atoms selected from nitrogen, oxygen and sulfur, and R₂, R₅, X₁ and Y₀ are as defined hereinbefore, can advantageously be prepared by reacting a compound of formula XIIe 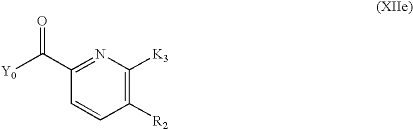
wherein R₂ and Y₀ are as defined hereinbefore, and K₃ is group capable of further functionalisation, such as chlorine, bromine, iodine or trifluoromethylsulfonyloxy, in accordance with process variant g) by means of a —C—C— linking Suzuki, Stille, Sonogashira or Heck reaction, in the presence of a noble metal catalyst having suitable ligands, e.g. PdCl₂(PPh₃)₂, Pd(PPh₃)₄, Pd₂(dba)₃, Pd(CH₃CN)₂(PPh₃)₂, Pd(OAc)₂, Rh, Cu, CuCl or CuI, and in the presence of a base, e.g. triethylamine, Hünig's base, sodium tert-butanolate, potassium tert-butanolate, sodium carbonate, potassium carbonate, caesium carbonate, potassium fluoride or caesium fluoride, and optionally further adjuvants, e.g. LiCl or Li₂CO₃, or an additional auxiliary catalyst, e.g. triphenylphosphine, tri(tert-butyl)phosphine, (Ph₃)₂PCH₂CH₂P(Ph₃)₂ or (Ph₃)₂PCH₂CH₂CH₂P(Ph₃)₂, with a boronic acid, e.g. of formula Xa or Xb
(HO)₂B-L₁₀-R₄ (Xa)
or
(HO)₂B-L₁₁-X₁—R₅ (Xb),
or with a tin compound of formula Xc or Xd
RₐR_{b}R_{c}Sn-L₁₀-R₄ (Xc)
or
RₐR_{b}R_{c}Sn-L₁₁-X₁—R₅ (Xd),
or with an ethynyl compound of formula Xe or Xf having a functionality in accordance with R₁
H—C≡C-L_{10b}-R₄ (Xe)
or
H—C≡C-L_{11b}-X₁—R₅ (Xf),
or with a vinyl compound of formula Xg or Xh having a functionality in accordance with R₁
H—CH═CH-L₁₀ₐ-R₄ (Xg)
or
H—CH═CH-L₁₁ₐ-X₁—R₅ (Xh),
wherein L₁₀, L₁₁, R₄, R₅ and X₁ are as defined hereinbefore, Rₐ, R_{b} and R_{c} are each independently of the others C₁-C₈alkyl and L₁₀ₐ, L_{10b}, L₁₁ₐ and L_{11b} are appropriate subgroups of the groups L₁₀ and L₁₁, such as especially a direct bond or a C₁-C₄alkylene group which may be substituted once, twice or three times by C₁-C₆alkyl, halogen, hydroxy, C₁-C₆alkoxy or by C₁-C₃alkoxy-C₁-C₃alkoxy, and wherein the reagents Xg and Xh can each result in one or more regio-isomeric products, for example

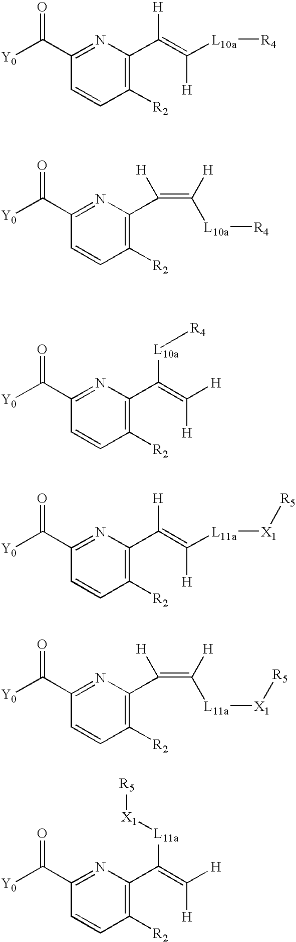

Those process sequences are described in greater detail in Reaction schemes 2 to 5 below.

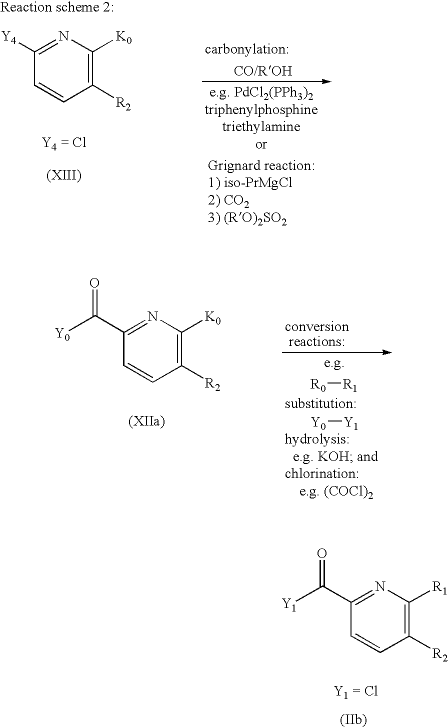

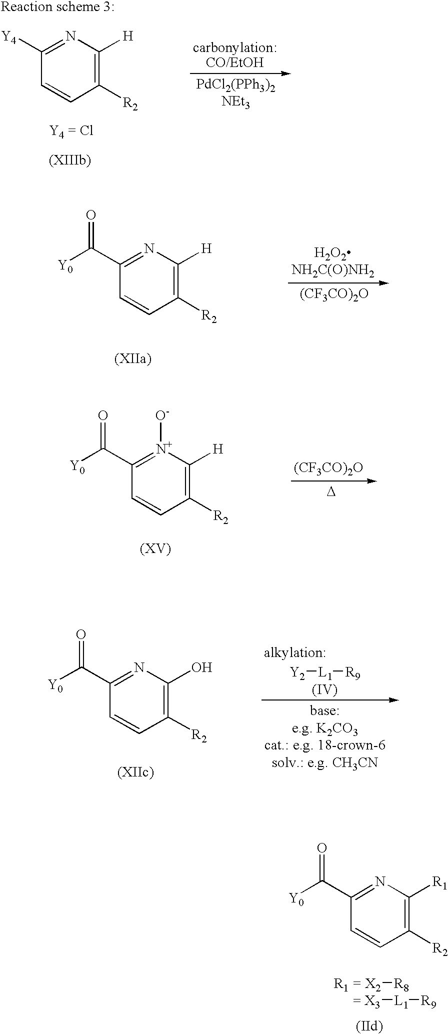

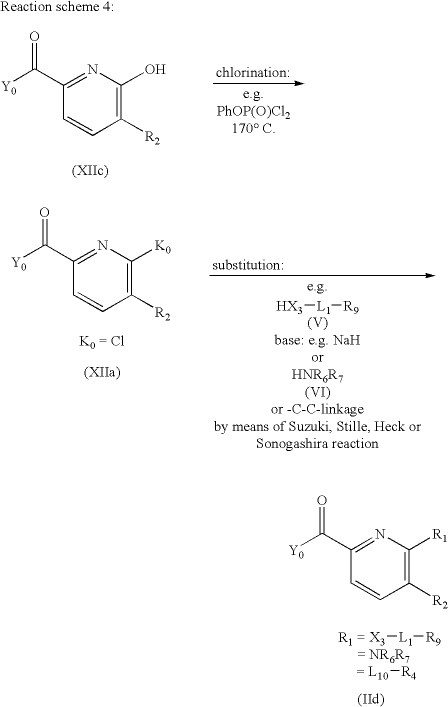

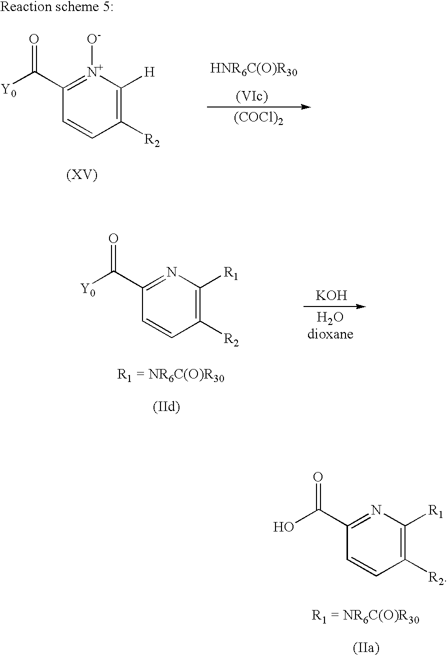

The compounds of formula XII 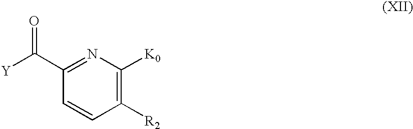
and XIIf 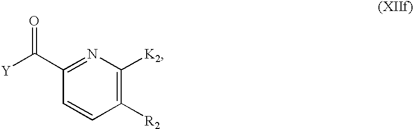
wherein K₀, K₂ and R₂ are as defined hereinbefore and Y is accordingly Y₀, hydroxy or Y₁, which are used as starting materials in the preparation of compounds of general formulae If and especially Ic, can likewise be prepared in accordance with the generally known methods or in accordance with the preparation processes c) to h) given above for formulae I and IIc.

The compounds of formula II used as starting materials 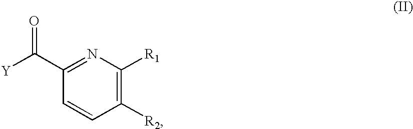
wherein R₁ and R₂ are as defined hereinbefore and Y is C₁-C₄alkoxy, benzyloxy, hydroxy, fluorine, chlorine, bromine, cyano or phenoxy which may be unsubstituted or substituted by an electron-withdrawing group, e.g. halogen, trifluoromethyl, nitro, cyano, C₁-C₄alkylcarbonyl, C₁-C₄alkoxycarbonyl or C₁-C₄alkylsulfonyl, are novel and the present Application relates also to the use thereof in the preparation of compounds of formula I. In compounds of formula II, Y is preferably C₁-C₄alkoxy, benzyloxy, hydroxy, chlorine or cyano.

The compounds of formula III used as starting materials are generally known. For example, diones of formula III 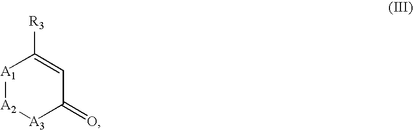
and of formula IIIa 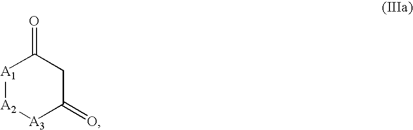
wherein A₁, A₂ and A₃ are as defined hereinbefore and R₃ is hydroxy or O⁻M⁺ wherein M⁺ is as defined hereinbefore, are known, for example, from DE-A-3902818, WO 00/39094, or they can be prepared in accordance with the methods described therein.

The starting materials of formulae IVa, IVb, IVc, V, Va, Vb, Vc, Vd, Ve, Vf, Vg, Vh, Vi, VI, VIa, VIb, VIc, VII, VIIa, VIII, VIIIa, IX, Xa, Xb, Xc, Xd, Xe, Xf, Xg, Xh, XIIIa, XIIIb are likewise generally known or they can be prepared analogously to known methods.

All the reactions according to the preparation processes a) to h) for forming compounds of formula I and also intermediates of formula II are advantageously carried out in aprotic and inert organic solvents. Such solvents are hydrocarbons, e.g. benzene, toluene, xylene or cyclohexane, chlorinated hydrocarbons, e.g. dichloromethane, chloroform, tetrachloromethane or chlorobenzene, ethers, e.g. diethyl ether, ethylene glycol dimethyl ether, diethylene glycol dimethyl ether, tetrahydrofuran or dioxane, nitriles, e.g. acetonitrile or propionitrile, and amides, e.g. N,N-dimethylformamide, diethylformamide or N-methylpyrrolidinone. The temperatures in those reactions are preferably from −20° C. to +120° C. When the reactions are exothermic, they can usually be carried out at room temperature. In order to shorten the reaction time or also in order to initiate the reaction, the reaction mixture may, where appropriate, be heated briefly up to its boiling point. Relatively new application techniques such as ultrasound and the use of microwaves are also highly suitable. It is often possible, especially when using microwaves, for the reaction times to be substantially reduced at relatively mild temperatures of from about 100° C. to about 150° C. The reaction times can likewise be shortened by adding a suitable base as reaction catalyst. Suitable bases are, especially, tertiary amines such as trimethylamine, triethylamine, quinuclidine, 1,4-diazabicyclo[2.2.2]octane, 1,5-diazabicyclo[4.3.0]non-5-ene and 1,5-diazabicyclo[5.4.0]-undec-7-ene. However, it is also possible to use inorganic bases, such as hydrides, e.g. sodium or calcium hydride, hydroxides, e.g. dry sodium or potassium hydroxide, carbonates, e.g. sodium or potassium carbonate, or hydrogen carbonates, e.g. sodium or potassium hydrogen carbonate.

In accordance with process a), preparation of the compounds of formulae I, If, and II, IIb and XII, wherein Y₁ and Y are chlorine, is carried out using a chlorinating agent, e.g. oxalyl chloride, thionyl chloride, phosgene, (1-chloro-2-methyl-propenyl)-dimethyl-amine, phosphorus pentachloride, phosphorus oxychloride or dichlorophosphate, preferably using oxalyl chloride. The reaction is preferably carried out in an inert, organic solvent, for example in an aliphatic, halogenated aliphatic, aromatic or halogenated aromatic hydrocarbon, e.g. n-hexane, benzene, toluene, xylenes, dichloromethane, 1,2-dichloroethane or chlorobenzene, at reaction temperatures in the range from −20° C. up to the reflux temperature of the reaction mixture, preferably at about 40-100° C., and in the presence of a catalytic amount of N,N-dimethylformamide. It can also, where appropriate, be carried out directly in the chlorinating agent used, without additional solvent.

The end products of formula I can be isolated in conventional manner by concentrating or evaporating off the solvent and can be separated and purified by recrystallising or by triturating the solid residue in solvents in which they are not readily soluble, such as ethers, aromatic hydrocarbons or chlorinated hydrocarbons, or by distillation or by means of column chromatography or by means of HPLC techniques using a suitable eluant.

The person skilled in the art will also be familiar with the order in which the reactions should be performed in order to avoid subsidiary reactions as far as possible. Unless synthesis is specifically aimed at the isolation of pure isomers, the product may be obtained in the form of a mixture of two or more isomers, e.g. chiral centres in the case of alkyl groups or cis/trans isomerism in the case of alkenyl groups or ‘E’ or ‘Z’ forms. All those isomers can be separated using methods known per se, e.g. chromatography or fractional crystallisation, or, by specifically controlling the reactions, a desired form can be produced in a relatively high concentration or in pure form.

The compounds of formula I according to the invention can be used as herbicides in unmodified form, that is to say as obtained in the synthesis, but they are generally formulated in various ways, using formulation adjuvants such as carriers, solvents and surface-active substances, to form herbicidal compositions. The formulations can be in various physical forms, e.g. in the form of dusting powders, gels, wettable powders, water-dispersible granules, water-dispersible tablets, effervescent tablet compacts, emulsifiable concentrates, micro-emulsifiable concentrates, oil-in-water emulsions, aqueous dispersions, dispersions in oil, suspoemulsions, water-soluble concentrates (having water or a water-miscible organic solvent as carrier), impregnated polymer films, or in other forms that are known, for example from the Manual on Development and Use of FAO Specifications for Plant Protection Products, 5th Edition, 1999. Those formulations either can be used directly or are diluted before use. The dilutions can be produced using, for example, water, liquid fertilisers, micro-nutrients, biological organisms, oil or solvents.

The formulations can be produced, for example, by mixing the active ingredient with the formulation adjuvants to obtain compositions in the form of finely divided solids, granules, spherules, solutions, dispersions or emulsions. The active ingredients can also be formulated with other adjuvants, such as finely divided solids, mineral oils, organic solvents, water, surface-active substances or combinations thereof. The active ingredients can also be contained in very fine microcapsules consisting of a polymer. Microcapsules contain the active ingredients in a porous carrier. This enables active ingredients to be released into the surroundings in controlled amounts. Microcapsules usually have a diameter of from 0.1 to 500 microns. They contain active ingredient in an amount of about from 25 to 95% by weight of the capsule weight. The active ingredients can be present in the form of a monolithic solid, in the form of fine particles in solid or liquid dispersion or in the form of a suitable solution. The encapsulating membranes comprise, for example, natural and synthetic gums, cellulose, styrene-butadiene copolymers, polyacrylonitrile, polyacrylate, polyester, polyamides, polyureas, polyurethane or chemically modified polymers and starch xanthates or other polymers known in this context to the person skilled in the art. Alternatively, it is possible for very fine microcapsules to be formed wherein the active ingredient is present in the form of finely divided particles in a solid matrix of a base substance, but in that case the microcapsules are not encapsulated.

The formulation adjuvants that are suitable for producing the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidinone, ethyl acetate, 2-ethylhexanol, ethylene carbonate, 1,1,1-trichloroethane, 2-heptanone, alpha-pinene, d-limonene, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol (PEG400), propionic acid, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol monomethyl ether, diethylene glycol monomethyl ether, methanol, ethanol, isopropanol, and alcohols of higher molecular weight such as amyl alcohol, tetrahydrofurfuryl alcohol, hexanol, octanol, etc., ethylene glycol, propylene glycol, glycerol, N-methyl-2-pyrrolidinone (NMP), and the like. Water is generally the carrier of choice for dilution of the concentrates. Suitable solid carriers are, for example, talc, titanium dioxide, pyrophyllite clay, silica, attapulgite clay, kieselguhr, chalk, calcium carbonate, bentonite, calcium montmorillonite, cottonseed husks, wheat flour, soybean flour, pumice, wood flour, ground walnut shells, lignin and similar substances such as are described, for example, in CFR 180.1001. (c) & (d).

A large number of surface-active substances can advantageously be used both in solid and in liquid formulations, especially in those which can be diluted with a carrier before application.

Surface-active substances can be anionic, cationic, non-ionic or polymeric, and they can be used as emulsifying agents, wetting agents or suspension agents or for other purposes. Typical surface-active substances include, for example, salts of alkyl sulfates, e.g. diethanolammonium lauryl sulfate; salts of alkylarylsulfonates, e.g. calcium dodecylbenzenesulfonate; addition products of alkylphenols and alkylene oxides, e.g. nonylphenol ethoxylates; addition products of alcohols and alkylene oxides, e.g. tridecylalcohol ethoxylates; soaps, e.g. sodium stearate; salts of alkylnaphthalenesulfonates, e.g. sodium dibutyl-naphthalenesulfonate; dialkyl esters of sulfosuccinate salts, e.g. sodium di(2-ethylhexyl)sulfosuccinate; sorbitol esters, e.g. sorbitol oleate; quaternary amines, e.g. lauryl trimethylammonium chloride, polyethylene glycol esters of fatty acids, e.g. polyethylene glycol stearate; block copolymers of ethylene oxide and propylene oxide; and salts of mono- and di-alkyl phosphate esters; and also further substances described, for example, in “McCutcheon's Detergents and Emulsifiers Annual” MC Publishing Corp., Ridgewood N.J., 1981.

Further adjuvants which can usually be used in herbicidal formulations include crystallisation inhibitors, viscosity-modifying substances, suspension agents, dyes, antioxidants, foaming agents, light-absorbing agents, mixing adjuvants, anti-foams, complex-formers, neutralising or pH-modifying substances and buffers, corrosion inhibitors, fragrances, wetting agents, take-up enhancers, micro-nutrients, plasticisers, glidants, lubricants, dispersants, thickening agents, antifreeze agents, microbicidal agents, and also liquid and solid fertilisers.

The formulations may also comprise additional active substances, e.g. further herbicides, herbicide safeners, plant growth regulators, fungicides and/or insecticides.

The compositions according to the invention may additionally include an additive comprising an oil of vegetable or animal origin, a mineral oil, alkyl esters thereof or mixtures of such oils and oil derivatives. The amounts of oil additive used in the composition according to the invention are generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhône-Poulenc Canada Inc.), alkyl esters of oils of vegetable origin, for example the methyl derivatives, or an oil of animal origin, such as fish oil or beef tallow. A preferred additive e.g. NOV233 contains as active components essentially 80% by weight alkyl esters of fish oils and 15% by weight methylated rapeseed oil, and also 5% by weight of customary emulsifiers and pH modifiers.

Especially preferred oil additives comprise alkyl esters of C₈-C₂₂ fatty acids, the methyl derivatives of C₁₂-C₁₈ fatty acids, for example the methyl esters of lauric acid, palmitic acid and oleic acid, being especially important. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). These and other oil derivatives are also known from the Compendium of Herbicide Adjuvants, 5th Edition, Southern Illinois University, 2000.

The application and action of the oil additives can be further improved by combining them with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltrisiloxanes, which are commercially available, for example, as Silwet L-77®, and also perfluorinated surfactants. The concentration of surface-active substances in relation to the total additive is generally from 1 to 30% by weight. Examples of oil additives that consist of mixtures of oils or mineral oils or derivatives thereof with surfactants are Edenor ME SU®, Turbocharge® (Zeneca Agro, CA) and Actipron® (BP Oil UK Limited, GB).

Where appropriate, the mentioned surface-active substances can also be used alone, that is to say without oil additives, in the formulations.

The addition of an organic solvent to the oil additive/surfactant mixture can also bring about a further enhancement of action. Suitable solvents are, for example, Solvesso® (ESSO) and Aromatic Solvent® (Exxon Corporation). The concentration of such solvents can be from 10 to 80% by weight of the total weight. Such oil additives, which are present in admixture with solvents, are described, for example, in U.S. Pat. No. 4,834,908. A commercially available oil additive known therefrom is known by the name MERGE® (BASF Corporation). A further oil additive that is preferred according to the invention is SCORE® (Syngenta Crop Protection Canada).

In addition to the oil additives listed above, it is also possible, for the purpose of enhancing the action of the compositions according to the invention, to add formulations of alkyl pyrrolidones (e.g. Agrimax®) to the spray mixture. Formulations of synthetic latices, such as, for example, polyacrylamide, polyvinyl compounds or poly-1-p-menthene (e.g. Bond®, Courier® or Emerald®), can also be used for the purpose. Solutions comprising propionic acid, for example Eurogkem Pen-e-trate®, can also be admixed as action-enhancing agents with the spray mixture.

The herbicidal formulations generally contain from 0.1 to 99% by weight, especially from 0.1 to 95% by weight, of compound of formula I and from 1 to 99.9% by weight of a formulation adjuvant which preferably contains from 0 to 25% by weight of a surface-active substance. Whereas commercial products will preferably be formulated as concentrates, the end user will normally employ dilute formulations.

The rates of application of compounds of formula I may vary within wide limits and depend on the nature of the soil, the method of application (pre- or post-emergence; seed dressing; application to the seed furrow; no tillage application etc.), the crop plant, the weed or grass to be controlled, the prevailing climatic conditions, and other factors governed by the method of application, the time of application and the target crop. The compounds of formula I according to the invention are generally applied at a rate of from 1 to 2000 g/ha.

The invention relates also to a method of selectively controlling grasses and weeds in crops of useful plants, which comprises treating the useful plants or the area of cultivation or locus thereof with the compounds of formula I.

The weeds to be controlled may be either monocotyledonous or dicotyledonous weeds, such as, for example, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus, Abutilon, Sida, Xanthium, Amaranthus, Chenopodium, Ipomoea, Chrysanthemum, Galium, Viola and Veronica.

As crops of useful plants in which the composition according to the invention can be used there come into consideration especially cereals, cotton, soybeans, sugar beet, sugar cane, plantation crops, rape, maize and rice. Crops are to be understood as including those which have been made tolerant to herbicides or classes of herbicides (e.g. ALS-, GS-, EPSPS- and HPPD-inhibitors) by means of conventional breeding or genetic engineering methods. An example of a crop that has been made tolerant by conventional breeding methods to, for example, imidazolinones such as imazamox is Clearfield® summer rape (canola). Examples of crops made tolerant to herbicides by genetic engineering methods are maize varieties resistant to, for example, glyphosate or glufosinate, which are commercially available under the trade names RoundupReady® and LibertyLink®.

Useful plants are to be understood as expressly including pest-resistant and/or fungus-resistant transgenic useful plants.

In the context of the present invention, pest-resistant transgenic crop plants are to be understood as being those which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popliae; or insecticidal proteins from Bacillus thuringiensis, such as δ-endotoxins, e.g. CryIA(b), CryIA(c), CryIF, CryIF(a2), CryIIA(b), CryIIIA, CryIIIB(b1) or Cry9c, or vegetative insecticidal proteins (VIP), e.g. VIP1, VIP2, VIP3 or VIP3A; or insecticidal proteins of bacteria-colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins; plant lectins, such as pea lectins, barley lectins or snowdrop lectins; agglutinins; proteinase inhibitors, such as trypsine inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by S-endotoxins, for example CryIA(b), CryIA(c), CryIF, CryIF(a2), CryIIA(b), CryIIIA, CryIIIB(b1) or Cry9c, or vegetative insecticidal proteins (VIP), for example VIP1, VIP2, VIP3 or VIP3A, expressly also hybrid toxins, truncated toxins and modified toxins. Hybrid toxins are produced recombinantly by a new combination of different domains of those proteins (see, for example, WO 02/15701). An example of a truncated toxin is a truncated CryIA(b), which is expressed in Bt11 maize of Syngenta Seeds SAS, as described hereinbelow. In the case of modified toxins, one or more amino acids of the naturally occurring toxin is/are replaced. In such amino acid replacements, preferably non-naturally present protease recognition sequences are inserted into the toxin, such as, for example, in the case of CryIIIA055, a cathepsin-D-recognition sequence is inserted into a CryIIIA toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants provides the plants with tolerance to harmful insects. Such insects can occur in any taxonomic group of insects, but are especially commonly found in beetles (Coleoptera), two-winged insects (Diptera) and butterflies (Lepidoptera).

The following harmful insects from different taxonomic groups are especially common in maize crops:
Ostrinia nubilalis, European corn borer
Agrotis epsilon, black cutworm
Helicoverpa zea, corn earworm
Spodoptera frugiperda, fall armyworm
Diatraea grandiosella, southwestern corn borer
Elasmopalpus lignosellus, lesser cornstalk borer
Diatraea saccharalis, sugarcane borer
Diabrotica virgifera virgifera, western corn rootworm
Diabrotica longicornis barberi, northern corn rootworm
Diabrotica undecimpunctata howardi, southern corn rootworm
Melanotus spp., wireworms
Cyclocephala borealis, northern masked chafer (white grub)
Cyclocephala immaculate, southern masked chafer (white grub)
Popillia japonica, Japanese beetle
Chaetocnema pulicaria, corn flea beetle
Sphenophorus maidis, maize billbug

Rhopalosiphum maidis, corn leaf aphid
Anuraphis maidiradicis, corn root aphid
Blissus leucopterus leucopterus, chinch bug
Melanoplus femurrubrum, red-legged grasshopper
Melanoplus sanguinipes, migratory grasshopper
Hylemya platura, seedcorn maggot
Agromyza parvicornis, corn blotch leafminer
Anaphothrips obscurus, grass thrips
Solenopsis milesta, thief ant 1Tetranychus urticae, two-spotted spider mite

Transgenic plants containing one or more genes that code for an insecticidal resistance and express one or more toxins are known and some of them are commercially available. Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Boligard I® (cotton variety that expresses a CryIA(c) toxin); Boligard II® (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated CryIA(b) toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a CryIA(b) toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified CryIIIA toxin. This toxin is Cry3A055 modified by insertion of a cathepsin-D-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a CryIIIB(b1) toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIA(b) toxin obtained from Bacillus thuringiensis subsp. kurstaki which brings about tolerance to certain Lepidoptera, including the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

In the context of the present invention, fungus-resistant transgenic crop plants are to be understood as being those which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defense (so-called “plant disease resistance genes”, as described in WO 03/000906).

In the context of the present invention, pest-resistant and/or fungus-resistant transgenic useful plants include expressly those useful plants which, in addition to pest-resistance and/or fungus-resistance, also possess herbicide tolerance. Of the group of herbicide-tolerant useful plants, preference is given according to the invention to those having tolerance to glyphosate, glufosinate ammonium, ALS (acetolactate synthase) inhibitors, e.g. sulfonylureas, for example primisulfuron, prosulfuron and trifloxysulfuron, or bromoxynil, such as Bt11 maize or Herculex I® maize.

Areas of cultivation are areas of land on which the crop plants are already growing and also areas of land on which it is intended to grow those crop plants.

The following Examples further illustrate, but do not limit, the invention.

### PREPARATION EXAMPLES

### Example P1

### Preparation of 1-oxy-5-trifluoromethyl-pyridine-2-carboxylic acid ethyl ester

197 g (2.1 mol) of hydrogen peroxide in the form of the urea adduct are stirred into a solution of 132 g (0.6 mol) of 5-trifluoromethyl-pyridine-2-carboxylic acid ethyl ester in 1000 ml of 1,2-dichloroethane. 346 g (1.65 mol) of trifluoroacetic anhydride are slowly added over 2.5 hours at a temperature of −10° C., with cooling (CO₂/acetone bath). The reaction mixture is then stirred for 2 hours at a temperature of 0° C. and then for 12 hours at ambient temperature. The reaction mixture is then poured into ice-water and adjusted to pH 6-7 with 30% sodium hydroxide solution. The mixture is extracted several times with 1,2-dichloroethane, dried over sodium sulfate and concentrated to dryness by evaporation. Chromatography on silica gel (eluant:ethyl acetate/hexane 1:4) is carried out in order to separate off subsidiary products. After removal of the eluant, 98.4 g of 1-oxy-5-trifluoromethyl-pyridine-2-carboxylic acid ethyl ester (m.p. 64.5 to 65° C.) are obtained.

### Example P2

### Preparation of 6-hydroxy-5-trifluoromethyl-pyridine-2-carboxylic acid ethyl ester

450 ml of trifluoroacetic anhydride are added dropwise to a mixture of 77.6 g (0.33 mol) of 1-oxy-5-trifluoromethyl-pyridine-2-carboxylic acid ethyl ester in 900 ml of dimethylformamide at a temperature of 0° C. over 3.5 hours. The mixture is then heated to a temperature of from 45 to 50° C. and is stirred for 2.5 hours. The reaction mixture is then concentrated under reduced pressure (2.5 kPa). The oily residue is poured into ice-water and adjusted to pH 5.5 with 30% sodium hydroxide solution. A crystallised product is obtained which is filtered off, washed with water and dried in vacuo at a temperature of 80° C. 61.6 g (79.4%) of 6-hydroxy-5-trifluoromethyl-pyridine-2-carboxylic acid ethyl ester (m.p. 141-141.5° C.) are obtained.

### Example P3

### Preparation of 6-chloro-5-trifluoromethyl-pyridine-2-carboxylic acid ethyl ester

16.5 g (70 mmol) of 6-hydroxy-5-trifluoromethyl-pyridine-2-carboxylic acid ethyl ester in 20 ml of phenyl dichlorophosphate are heated in a pressure reactor for 30 minutes at a temperature of 170° C. The cooled reaction mixture is taken up in ethyl acetate, washed once with cold sodium chloride solution, dried over sodium sulfate and then concentrated. To remove phosphate-containing constituents, the residue that remains behind is chromatographed on silica gel (eluant:ethyl acetate/hexane 1:4) and is then concentrated to dryness by evaporation. 16.2 g (91.3%) of 6-chloro-5-trifluoromethyl-pyridine-2-carboxylic acid ethyl ester are obtained in the form of an oil; ¹H NMR (CDCl₃): 8.17, m, 2H, 4.52, q, 2H, 1.44, t, 3H.

### Example P4

### Preparation of 6-(morpholin-4-yl)-5-trifluoromethyl-pyridine-2-carboxylic acid ethyl ester

6.45 g (25 mmol) of 6-chloro-5-trifluoromethyl-pyridine-2-carboxylic acid ethyl ester are heated at a temperature of 110° C. for 1 hour in the presence of 5.5 g (63 mmol) of morpholine and a catalytic amount of 4-N,N-dimethylaminopyridine in 50 ml of N-methylpyrrolidone. The reaction mixture is adjusted to pH 4 with dilute hydrochloric acid, extracted with ethyl acetate, dried over magnesium sulfate and then concentrated. In order to remove polar subsidiary products, the residue is chromatographed on silica gel and then concentrated to dryness by evaporation, yielding 7.08 g of 6-(morpholin-4-yl)-5-trifluoromethyl-pyridine-2-carboxylic acid ethyl ester in the form of an oil; ¹H NMR (CDCl₃): 7.97, d, 1H, 7.68, d, 1H, 4.42, q, 2H, 3.83, m, 4H, 3.40, m, 4H, 1.42, t, 3H.

### Example P5

### Preparation of 6-(morpholin-4-yl)-5-trifluoromethyl-pyridine-2-carboxylic acid

7 g (23 mmol) of 6-(morpholin-4-yl)-5-trifluoromethyl-pyridine-2-carboxylic acid ethyl ester are added to a mixture of 30 ml of dioxane and 25 ml of water in the presence of 1.55 g of potassium hydroxide and the reaction mixture is stirred at ambient temperature for 30 minutes. The reaction mixture is then adjusted to pH 3 and extracted with ethyl acetate, dried over sodium sulfate and concentrated. Addition of hexane causes 6-(morpholin-4-yl)-5-trifluoromethyl-pyridine-2-carboxylic acid (yield 93.2%) to precipitate out (m.p.: 116-117° C.).

### Example P6

### Preparation of 6-(morpholin-4-yl)-5-trifluoromethyl-pyridine-2-carboxylic acid chloride

0.83 g (3 mmol) of 6-(morpholin-4-yl)-5-trifluoromethyl-pyridine-2-carboxylic acid and 0.46 g (3.6 mmol) of oxalyl chloride in 10 ml of dichloromethane are heated at boiling temperature for 15 minutes in the presence of a drop of dimethylformamide. The solution is then concentrated by evaporation, yielding 6-(morpholin-4-yl)-5-trifluoromethyl-pyridine-2-carboxylic acid chloride in the form of a crystalline product (m.p.: 72-73° C.).

### Example P7

### Preparation of 6-(acetyl-methyl-amino)-5-trifluoromethyl-pyridine-2-carboxylic acid ethyl ester

At a temperature of 0° C., with cooling, a solution of 1.46 g (11.5 mmol) of oxalyl chloride in ml of dichloromethane is added dropwise to a solution of 0.84 g (11.5 mmol) of N-methylacetamide and 2.45 g (22.9 mmol) of lutidine in 40 ml of dichloromethane. After 20 minutes, 2.45 g (10.4 mmol) of 1-oxy-5-trifluoromethyl-pyridine-2-carboxylic acid ethyl ester dissolved in 5 ml of dichloromethane are added. The reaction mixture is heated to ambient temperature and then heated at boiling temperature for 1 hour. The reaction mixture is then extracted with water against dichloromethane, dried over MgSO₄ and concentrated to dryness by evaporation. The resulting residue is chromatographed on silica gel (eluant:ethyl acetate/hexane 3:7), 6-(acetyl-methyl-amino)-5-trifluoromethyl-pyridine-2-carboxylic acid ethyl ester being isolated as main component (m.p.: 145-145.5° C.).

### Example P8

### Preparation of 6-([1,3]dioxolan-2-ylmethoxy)-5-trifluoromethyl-pyridine-2-carboxylic acid ethyl ester

1.88 g (8 mmol) of 6-hydroxy-5-trifluoromethyl-pyridine-2-carboxylic acid ethyl ester and 1.47 g (8 mmol) of 2-bromomethyl-1,3-dioxolane in 30 ml of acetonitrile are heated at reflux temperature in the presence of 1.22 g (8.8 mmol) of potassium carbonate and catalytic amounts of potassium iodide and 18-crown-6 for 6 hours. The reaction mixture is then extracted with ethyl acetate against water and dilute acid at pH 3, dried over sodium sulfate and concentrated by evaporation. The residue is chromatographed on silica gel (eluant:ethyl acetate/hexane 15:85), yielding 6-([1,3]dioxolan-2-ylmethoxy)-5-trifluoromethyl-pyridine-2-carboxylic acid ethyl ester in the form of an oil; ¹H NMR (CDCl₃): 8.00, d, 1H, 7.76, d, 1H, 5.40, t, 1H, 4.61, d, 2H, 4.42, q, 2H, 4.09, m, 2H, 3.93, m, 2H, 1.42, t, 3H.

### Example P9

### Preparation of 6-(tetrahydro-furan-3-yloxy)-5-trifluoromethyl-pyridine-2-carboxylic acid ethyl ester

2.53 g (14.5 mmol) of azodicarboxylic acid diethyl ester (DEAD) are added dropwise to a solution of 2.35 g (10 mmol) of 6-hydroxy-5-trifluoromethyl-pyridine-2-carboxylic acid ethyl ester and 3.93 g (15 mmol) of triphenylphosphine in 30 ml of dimethoxyethane, the temperature being maintained at a maximum of 35° C. After 1 hour at ambient temperature, the reaction mixture is concentrated to dryness by evaporation. The residue is chromatographed on silica gel (eluant:ethyl acetate/hexane 1:4). 2.85 g (93.4%) of 6-(tetrahydrofuran-3-yloxy)-5-trifluoromethyl-pyridine-2-carboxylic acid ethyl ester are obtained (m.p.: 45-45.5° C.).

### Example P10

### 6-Methyl-5-trifluoromethyl-pyridinecarboxylic acid ethyl ester

6.9 g (6 mmol) of tetrakis(triphenylphosphine)palladium and 8.3 g (66 mmol) of 2,4,6-trimethyl-cyclotriboroxane are added to a solution of 15.2 g (60 mmol) of 6-chloro-5-trifluoromethyl-pyridinecarboxylic acid ethyl ester and 33.1 g (0.24 mol) of potassium carbonate in 150 ml of dioxane and the mixture is then heated for 2.5 hours at reflux temperature. The end point of the reaction is determined by thin-layer chromatography. The reaction mixture is cooled, poured into ice-water and is then adjusted to pH 5 with concentrated hydrochloric acid. To separate off solid constituents, a filtration aid (Hyflo®) is added and filtered off. The filtrate is extracted with ethyl acetate. The filtrate, dried over sodium sulfate, is concentrated to dryness by evaporation and chromatographed on silica gel (eluant:ethyl acetate/hexane 7.5:92.5). 11.28 g (87.8%) of 6-methyl-5-trifluoromethyl-pyridinecarboxylic acid ethyl ester are obtained in the form of an oil; ¹H NMR (CDCl₃): 8.05, “s”, 2H, 4.48, q, 2H, 2.31, 2, 3H, 1.42, t, 3H.

### Example P11

### 6-Bromomethyl-5-trifluoromethyl-pyridine-2-carboxylic acid ethyl ester

A catalytic amount of aza,aza-diisobutyronitrile is added to 1 g (4.3 mmol) of 6-methyl-5-trifluoromethyl-pyridine-2-carboxylic acid ethyl ester and 0.92 g of N-bromo-succinimide in 20 ml of carbon tetrachloride. The mixture is heated at reflux temperature using a light source (200 W lamp). Cooling is then carried out and the reaction product is filtered off and purified using an HPLC technique (eluant:ethyl acetate/hexane 1:4). 6-Bromomethyl-5-trifluoromethyl-pyridine-2-carboxylic acid ethyl ester is obtained; ¹H NMR (CDCl₃): 6.14, “s”, 2H, 4.78, s, 2H, 4.49, q, 2H, 1.45, t, 3H.

### Example P12

### 6-(2-Methoxy-ethoxymethyl)-5-trifluoromethyl-pyridine-2-carboxylic acid

0.6 g (2 mmol) of 6-bromomethyl-5-trifluoromethyl-pyridine-2-carboxylic acid ethyl ester, dissolved in 3 ml of tetrahydrofuran, is introduced into 0.25 g (5.8 mmol) of sodium hydride (as a 55% dispersion in oil) in 10 ml of dry tetrahydrofuran and stirring is carried out for 2 hours at room temperature. The end point of the reaction is determined by means of thin-layer chromatography. Water is then added. After hydrolysis of the ester group is complete (again demonstrated by means of thin-layer chromatography), extraction with diethyl ether is carried out. The aqueous phase, which contains the reaction product, is then adjusted to pH 2 with hydrochloric acid. Extraction with ethyl acetate is then carried out, followed by drying over sodium sulfate and concentration to dryness by evaporation. 6-(2-Methoxyethoxymethyl)-5-trifluoromethyl-pyridine-2-carboxylic acid is obtained; ¹H NMR (CDCl₃): 8.26, d, 1H, 8.17, d, 1H, 7.3, b, OH; 4.96, s, 2H, 3.91, m, 2H, 3.71, m, 2H, 3.48, s, 3H.

### Example P13

### 2-(6-Thiomorpholin-4-yl-5-trifluoromethyl-pyridine-2-carbonyl)-cyclohexane-1,3-dione

0.31 g (1 mmol) of 6-thiomorpholin-4-yl-5-trifluoromethyl-pyridine-2-carboxylic acid chloride, prepared with oxalyl chloride, is added to 0.11 g (1 mmol) of cyclohexane-1,3-dione and 0.25 g (2.5 mmol) of triethylamine in 15 ml of acetonitrile and the reaction mixture is stirred at room temperature for 2 hours. 2 drops of acetone cyanohydrin are then added and the mixture is stirred for 12 hours. The reaction mixture is taken up in ethyl acetate and extracted with dilute hydrochloric acid at pH 3. After concentration of the organic phase by evaporation, the residue that remains behind is chromatographed on silica gel (eluant:ethyl acetate/methanol/triethylamine 85:10:5). After concentration by evaporation, the triethylammonium salt of 2-(6-thiomorpholin-4-yl-5-trifluoromethyl-pyridine-2-carbonyl)-cyclohexane-1,3-dione is obtained in the form of a resinous product. In order to free the desired end product, the resinous product is taken up in ethyl acetate, extracted again with dilute hydrochloric acid, dried over sodium sulfate and again concentrated to dryness by evaporation. After recrystallization with ethyl acetate and hexane, 2-(6-thiomorpholin-4-yl-5-trifluoromethyl-pyridine-2-carbonyl)-cyclohexane-1,3-dione is obtained in crystalline form (m.p.: 106-106.5° C.).

### Example P14

### 2-(6-Pyrazol-1-yl-5-trifluoromethyl-pyridine-2-carbonyl)-cyclohexane-1,3-dione

0.32 g (1 mmol) of 2-(6-chloro-5-trifluoromethyl-pyridine-2-carbonyl)-cyclohexane-1,3-dione and 82 mg (1.2 mmol) of pyrazole are added in succession at room temperature to 0.11 g of a 55% sodium hydride dispersion (2.5 mmol) in 8 ml of N-methylpyrrolidone. The reaction mixture is stirred for 1.5 hours at a temperature of 120° C. When the reaction mixture is cool, water is added and the mixture is adjusted to pH 2 and extracted with ethyl acetate. The reaction mixture is then concentrated to dryness by evaporation. The residue is chromatographed on silica gel (eluant:ethyl acetate/hexane/formic acid 49.5:49.5:1). 2-(6-Pyrazol-1-yl-5-trifluoromethyl-pyridine-2-carbonyl)-cyclohexane-1,3-dione is obtained in the form of a resinous product; ¹H NMR (CDCl₃): 15.78, b, OH, 8.30, d, 1H, 8.07, d, 1H, 7.69, d, 1H, 7.54, d, 1H, 6.44, m, 1H, 2.80, m, 2H, 2.48, m, 2H, 2.10, m, 2H.

### Example P15

### 2-[6-(4-Methoxy-phenyl)-5-trifluoromethyl-pyridine-2-carbonyl]-cyclohexane-1,3-dione

16 mg (0.05 mmol) of 2-(6-chloro-5-trifluoromethyl-pyridine-2-carbonyl)-cyclohexane-1,3-dione and 9 mg (0.05 mmol) of p-methoxyphenyl-boronic acid are dissolved in 0.98 ml of dimethoxyethane/water/ethanol (7:3:2) and, in a microwave oven, heated at 140° C. for 400 seconds in the presence of a catalytic amount of bis(triphenyl)phosphine palladium dichloride and 49 mg (0.15 mmol) of caesium carbonate. When the reaction solution is cool, it is transferred to a deep-well plate. By supplying a stream of nitrogen, the volatile constituents of the reaction solution are removed by evaporation. 0.1 ml of trifluoroacetic acid and 0.4 ml of dimethylformamide are added to the residue and stirring is carried out for 10 minutes. The soluble component is purified by means of HPLC under reversed-phase conditions (eluant:gradient of water and acetonitrile). 2-[6-(4-Methoxy-phenyl)-5-trifluoromethyl-pyridine-2-carbonyl]-cyclohexane-1,3-dione is obtained in the form of a resin.

### Example P16

### 5-Trifluoromethyl-6-vinyl-pyridine-2-carboxylic acid ethyl ester

3 g (11.8 mmol) of chloro-5-trifluoromethyl-pyridine-2-carboxylic acid ethyl ester are dissolved in degassed dioxane, and 4.13 g (13.02 mmol) of tributylvinyltin are added. After the addition of 273 mg (0.236 mmol) of Pd(Ph₃P)₄, the reaction mixture is stirred for 20 hours at 95° C. The reaction mixture is concentrated and is chromatographed on silica gel (eluant:ethyl acetate/isohexane 1:5). 2 g of 5-trifluoromethyl-6-vinyl-pyridine-2-carboxylic acid ethyl ester are obtained. ¹H NMR (CDCl₃): 8.07 (d), J=8.3 Hz, 1H, 8.034 (d), J=8.3 Hz, 1H, 7.06-7.18 (m), Jab=16.6 Hz, Jac=10.5 Hz, 1Ha; 6.77 (dd), Jba=16.6 Hz, Jbc=2 Hz, 1Hb; 5.74 (dd), Jca=10.5 Hz, Jcb=2 Hz, 1Hc; 4.487 (q), 2H, 1.455 (t), 3H.

### Example P17

### 6-(1-Ethoxy-vinyl)-5-trifluoromethyl-pyridine-2-carboxylic acid ethyl ester

1.6 g (6.31 mmol) of chloro-5-trifluoromethyl-pyridine-2-carboxylic acid ethyl ester are dissolved in 32 ml of degassed dioxane, and 2.5 g (6.92 mmol) of 1-ethoxy-1-tributylvinyltin are added. After the addition of 146 mg (0.126 mmol) of Pd(Ph₃P)₄, the reaction mixture is stirred for 20 hours at 95° C. The reaction mixture is concentrated and is chromatographed on silica gel (eluant:ethyl acetate/isohexane 1:5). 0.9 g of 6-(1-ethoxy-vinyl)-5-trifluoromethyl-pyridine-2-carboxylic acid ethyl ester are obtained. ¹H NMR (CDCl₃): 8.148 (s), 2H, 4.732 (d), Jab=3 Hz, 1Ha; 4.509 (d), Jba=3 Hz, 1Hb; 4.497 (q), CH₂O (ester); 2H, 3.952 (q), CH₂O (ether); 2H 1.436 (t), CH₃ (ester), 3H, 1.376 (t), CH₃ (ether).

By that means it is possible, for example, also for the compounds of formula I listed in the following Table to be prepared, those compounds that are defined as an oil, resin, wax or amorphous solid at least having been characterised in pure form by means of ¹H NMR (nuclear resonance spectroscopy) and/or MS (mass spectrometry).

In general, substituents R₁ are bonded to the pyridyl ring by way of the free valence at the atom on the left-hand side of the substituent R₁, as in the case, for example, of compound 1.001. In that compound, the substituent R₁═OCH₂OCH₃ is bonded to the pyridyl ring by way of the free valence of the oxygen atom on the left-hand side of the substituent. In individual cases, the linking atom is, in addition, marked by means of an apostrophe, as in the case, for example, of compound no. 1.137.

In the case of cyclic substituents R₁ that are bonded directly to the pyridyl ring, the ring atom of the cyclic substituent R₁ linked to the pyridyl is given special emphasis by means of an apostrophe, as, for example, in

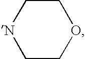

where the linkage site is located at the nitrogen atom denoted by ′N.

TABLE 1
Compounds of formula IA

| | |
|---|---|
| | (IA) |
| 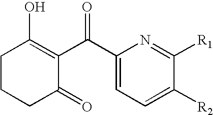 | |

| Ex. No. | R₁ | R₂ | Physical properties |
|---|---|---|---|
| 1.001 | OCH₂OCH₃ | CF₃ | |
| 1.002 | OCH₂OCH₂CH₃ | CF₃ | |
| 1.003 | OCH₂CH₂OCH₃ | CF₃ | resin |
| 1.004 | OCH₂CH₂OCH₂CH₃ | CF₃ | |
| 1.005 | OCH₂CH₂CH₂OCH₃ | CF₃ | |
| 1.006 | OCH(CH₃)CH₂OCH₃ | CF₃ | |
| 1.007 | OCH₂CH₂OCH₂CH═CH₂ | CF₃ | |
| 1.008 | OCH₂CH₂OCH₂C≡CH | CF₃ | |
| 1.009 | OCH₂CH₂O-benzyl | CF₃ | |
| 1.010 | OCH₂CH₂ON═C(CH₃)₂ | CF₃ | |
| 1.011 | OCH₂CH₂OCH₂CH₂OCH₃ | CF₃ | |
| 1.012 | OCH₂CH(OCH₃)₂ | CF₃ | resin |
| 1.013 | OCH₂CH(OCH₂CH₃)₂ | CF₃ | |
| 1.014 | 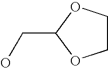 | CF₃ | |
| 1.015 | 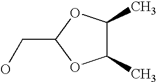 | CF₃ | |
| 1.016 | 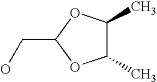 | CF₃ | |
| 1.017 | 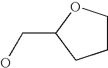 | CF₃ | |
| 1.018 | 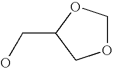 | CF₃ | |
| 1.019 | 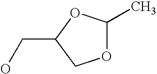 | CF₃ | |
| 1.020 | 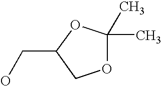 | CF₃ | |
| 1.021 | 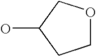 | CF₃ | resin |
| 1.022 | O-benzyl | CF₃ | resin |
| 1.023 | OCH₂CH₂SCH₃ | CF₃ | |
| 1.024 | OCH₂CH₂SCH₂CH₃ | CF₃ | |
| 1.025 | OCH₂CH₂S(O)₂CH₃ | CF₃ | |
| 1.026 | OCH₂CH₂S(O)₂CH₂CH₃ | CF₃ | |
| 1.027 | SCH₂CH₂OCH₃ | CF₃ | |
| 1.028 | SCH₂CH₂OCH₂CH₃ | CF₃ | |
| 1.029 | OCH₂CH₂OC(O)CH₃ | CF₃ | |
| 1.030 | OCH₂CH₂OC(O)-phenyl | CF₃ | |
| 1.031 | OCH₂CH₂OC(O)OCH₂CH₃ | CF₃ | |
| 1.032 | OCH₂CH₂OC(O)NHCH₂CH₃ | CF₃ | |
| 1.033 | OCH₂CH₂NH₂ | CF₃ | |
| 1.034 | OCH₂CH₂NHC(O)CH₃ | CF₃ | |
| 1.035 | OCH₂CH₂NHC(O)CH₂CH₃ | CF₃ | |
| 1.036 | OCH₂CH₂NHC(O)CH(CH₃)₂ | CF₃ | |
| 1.037 | OCH₂CH₂NHC(O)-cyclopropyl | CF₃ | |
| 1.038 | OCH₂CH₂NHC(O)C(CH₃)₃ | CF₃ | |
| 1.039 | OCH₂CH₂NHC(O)-phenyl | CF₃ | |
| 1.040 | OCH₂CH₂NHC(O)OCH₃ | CF₃ | |
| 1.041 | OCH₂CH₂NHC(O)OCH₂CH₃ | CF₃ | resin |
| 1.042 | OCH₂CH₂NHC(O)NHCH₃ | CF₃ | |
| 1.043 | OCH₂CH₂NHC(O)NHCH₂CH₃ | CF₃ | |
| 1.044 | OCH₂CH₂NHC(O)N(CH₃)₂ | CF₃ | |
| 1.045 | OCH₂CH₂NHC(O)N(CH₂CH₃)₂ | CF₃ | |
| 1.046 | NHCH₃ | CF₃ | |
| 1.047 | NHCH₂CH₃ | CF₃ | |
| 1.048 | NHCH₂CH₂CH₃ | CF₃ | |
| 1.049 | NHCH₂CH₂CH₂CH₃ | CF₃ | |
| 1.050 | NHCH(CH₃)₂ | CF₃ | |
| 1.051 | NHC(CH₃)₃ | CF₃ | |
| 1.052 | NHCH₂-cyclopropyl | CF₃ | |
| 1.053 | NH-phenyl | CF₃ | |
| 1.054 | NH-benzyl | CF₃ | |
| 1.055 | NHCH₂CH═CH₂ | CF₃ | |
| 1.056 | NHCH₂C≡CH | CF₃ | |
| 1.057 | N(CH₂CH═CH₂)₂ | CF₃ | |
| 1.058 | N(CH₂C≡CH)₂ | CF₃ | |
| 1.059 | N(CH₃)₂ | CF₃ | viscous oil |
| 1.060 | N(CH₂CH₃)₂ | CF₃ | viscous oil |
| 1.061 | N(CH₂CH₂CH₃)₂ | CF₃ | |
| 1.062 | N(CH₂CH₂CH₂CH₃)₂ | CF₃ | |
| 1.063 | NHCH₂CH₂OH | CF₃ | |
| 1.064 | NHCH₂CH₂OCH₃ | CF₃ | resin |
| 1.065 | NHCH(CH₃)CH₃OCH₃ | CF₃ | |
| 1.066 | NHCH₂CH(OCH₃)₂ | CF₃ | |
| 1.067 | NHCH₂CH(OCH₂CH₃)₂ | CF₃ | |
| 1.068 | 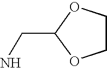 | CF₃ | |
| 1.069 | NHCH₂C(O)OCH₃ | CF₃ | |
| 1.070 | NHCH(CH₃)C(O)OCH₃ | CF₃ | |
| 1.071 | NHCH₂C(O)OCH₂CH₃ | CF₃ | |
| 1.072 | NHCH(CH₃)C(O)OCH₂CH₃ | CF₃ | |
| 1.073 | 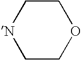 | CF₃ | resin |
| 1.074 | 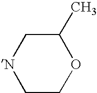 | CF₃ | |
| 1.075 | 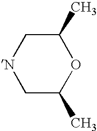 | CF₃ | m.p.: 82-83° C. |
| 1.076 | 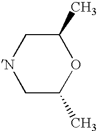 | CF₃ | |
| 1.077 | 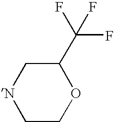 | CF₃ | |
| 1.078 | 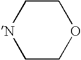 | CHF₂ | |
| 1.079 | 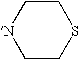 | CF₃ | m.p.: 106-106.5° C. [P13] |
| 1.080 | 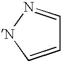 | CF₃ | resin, [P14] |
| 1.081 | 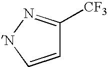 | CF₃ | m.p. 137-138° C. |
| 1.082 | 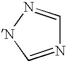 | CF₃ | resin |
| 1.083 | 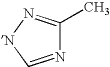 | CF₃ | |
| 1.084 | 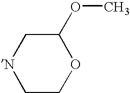 | CF₃ | |
| 1.085 | 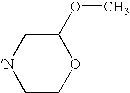 | CHF₂ | |
| 1.086 | 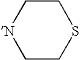 | CHF2 | |
| 1.087 | 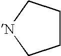 | CHF₃ | |
| 1.088 | N(CH₃)O(O)H | CF₃ | |
| 1.089 | N(CH₃)C(O)CH₃ | CF₃ | m.p. 130-131° C. |
| 1.090 | N(CH₃)O(O)CH₂CH₃ | CF₃ | m.p. 120-121° C. |
| 1.091 | N(CH₃)C(O)-phenyl | CF₃ | m.p. 141-142° C. |
| 1.092 | N(CH₃)O(O)-benzyl | CF₃ | resin |
| 1.093 | N(CH₂CH₃)C(O)CH₃ | CF₃ | |
| 1.094 | 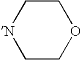 | OCH₂CF₃ | resin |
| 1.095 | Cl | CF₃ | amorphous crystals |
| 1.096 | OCH₃ | CF₃ | m.p. 80-81° C. |
| 1.097 | CH₂OH | CF₃ | |
| 1.098 | CH₂Cl | CF₃ | |
| 1.099 | CH₂Br | CF₃ | |
| 1.100 | CH₂OSO₂CH₃ | CF₃ | |
| 1.101 | CH₂OC(O)CH₃ | CF₃ | |
| 1.102 | CH₂OC(O)C(CH₃)₃ | CF₃ | |
| 1.103 | CH₂OC(O)phenyl | CF₃ | |
| 1.104 | CH₂OC(O)OCH₂CH₃ | CF₃ | |
| 1.105 | CH₂OCH₃ | CF₃ | |
| 1.106 | CH₂OCH₂CH₃ | CF₃ | |
| 1.107 | CH₂CH₂OCH₃ | CF₃ | |
| 1.108 | CH₂CH₂OCH₂CH₃ | CF₃ | |
| 1.109 | CH₂CH₂CH₂OCH₃ | CF₃ | |
| 1.110 | CH(CH₃)CH₂OCH₃ | CF₃ | |
| 1.111 | CH₂OCH₂CH═CH₂ | CF₃ | |
| 1.112 | CH₂OCH₂C≡CH | CF₃ | |
| 1.113 | CH₂OCH₂C≡CCH₃ | CF₃ | |
| 1.114 | CH₂OCH₂CH₂C≡CH | CF₃ | |
| 1.115 | CH₂OCH₂CH₂C≡CCH₃ | CF₃ | |
| 1.116 | CH₂O-benzyl | CF₃ | |
| 1.117 | CH₂OCH₂CF₃ | CF₃ | |
| 1.118 | CH₂OCH₂CH₂F | CF₃ | |
| 1.119 | CH₂OCH₂CH₂Cl | CF₃ | |
| 1.120 | CH₂OCH₂CH₂Br | CF₃ | |
| 1.121 | CH₂OCH₂CH₂C≡N | CF₃ | |
| 1.122 | CH₂OCH₂C≡N | CF₃ | |
| 1.123 | CH₂OCH₂OCH₃ | CF₃ | |
| 1.124 | CH₂OCH₂OCH₂CH₃ | CF₃ | |
| 1.125 | CH₂OCH₂CH₂OH | CF₃ | |
| 1.126 | CH₂OCH₂CH₂OCH₃ | CF₃ | resin, [P12] |
| 1.127 | CH₂OCH₂CH₂OCH₂CH₃ | CF₃ | |
| 1.128 | CH₂OCH₂CH₂CH₂OCH₃ | CF₃ | |
| 1.129 | CH₂OCH(CH₃)CH₂OCH₃ | CF₃ | |
| 1.130 | CH₂OCH₂CH₂OCH₂CH═CH₂ | CF₃ | |
| 1.131 | CH₂OCH₂CH₂OCH₂C≡CH | CF₃ | |
| 1.132 | CH₂OCH₂CH₂O-benzyl | CF₃ | |
| 1.133 | CH₂OCH₂CH₂ON═C(CH₃)₂ | CF₃ | |
| 1.134 | CH₂OCH₂CH₂OCH₂CH₂OCH₃ | CF₃ | |
| 1.135 | CH₂OCH₂CH(OCH₃)₂ | CF₃ | |
| 1.136 | CH₂OCH₂CH(OCH₂CH₃)₂ | CF₃ | |
| 1.137 | 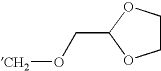 | CF₃ | |
| 1.138 | 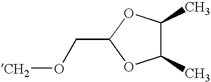 | CF₃ | |
| 1.139 | 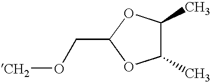 | CF₃ | |
| 1.140 | 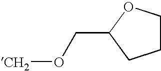 | CF₃ | |
| 1.141 | 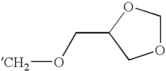 | CF₃ | |
| 1.142 | 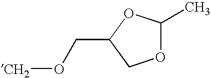 | CF₃ | |
| 1.143 | 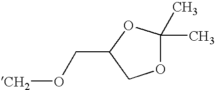 | CF₃ | |
| 1.144 | 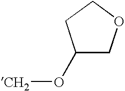 | CF₃ | |
| 1.145 | 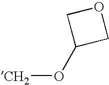 | CF₃ | |
| 1.146 | 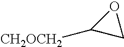 | CF₃ | |
| 1.147 | CH₂OCH₂CH₂SCH₃ | CF₃ | |
| 1.148 | CH₂OCH₂CH₂SCH₂CH₃ | CF₃ | |
| 1.149 | CH₂OCH₂CH₂S(O)₂CH₃ | CF₃ | |
| 1.150 | CH₂OCH₂CH₂S(O)₂CH₂CH₃ | CF₃ | |
| 1.151 | CH₂SCH₂CH₂OCH₃ | CF₃ | |
| 1.152 | CH₂SCH₂CH₂OCH₂CH₃ | CF₃ | |
| 1.153 | CH₂OCH₂CH₂OC(O)CH₃ | CF₃ | |
| 1.154 | CH₂OCH₂CH₂OC(O)-phenyl | CF₃ | |
| 1.155 | CH₂OCH₂CH₂OC(O)OCH₂CH₃ | CF₃ | |
| 1.156 | CH₂OCH₂CH₂OC(O)NHCH₂CH₃ | CF₃ | |
| 1.157 | CH₂OCH₂CH₂NH₂ | CF₃ | |
| 1.158 | CH₂OCH₂CH₂NHC(O)CH₃ | CF₃ | |
| 1.159 | CH₂OCH₂CH₂NHC(O)CH₂CH₃ | CF₃ | |
| 1.160 | CH₂OCH₂CH₂NHC(O)CH(CH₃)₂ | CF₃ | |
| 1.161 | CH₂OCH₂CH₂NHC(O)-cyclopropyl | CF₃ | |
| 1.162 | CH₂OCH₂CH₂NHC(O)C(CH₃)₃ | CF₃ | |
| 1.163 | CH₂OCH₂CH₂NHC(O)-phenyl | CF₃ | |
| 1.164 | CH₂OCH₂CH₂NHC(O)OCH₃ | CF₃ | |
| 1.165 | CH₂OCH₂CH₂NHC(O)OCH₂CH₃ | CF₃ | |
| 1.166 | CH₂OCH₂CH₂NHC(O)NHCH₃ | CF₃ | |
| 1.167 | CH₂OCH₂CH₂NHC(O)NHCH₂CH₃ | CF₃ | |
| 1.168 | CH₂OCH₂CH₂NHC(O)N(CH₃)₂ | CF₃ | |
| 1.169 | CH₂OCH₂CH₂NHC(O)N(CH₂CH₃)₂ | CF₃ | |
| 1.170 | CH₂N(SO₂CH₃)CH₃ | CF₃ | |
| 1.171 | CH₂N(SO₂CH₃)CH₂CH₃ | CF₃ | |
| 1.172 | CH₂N(SO₂CH₂CH₃)CH₂CH₃ | CF₃ | |
| 1.173 | CH₂N(SO₂CH₃)CH₂CF₃ | CF₃ | |
| 1.174 | CH₂N(SO₂CH₃)CH₂CHOCH₃ | CF₃ | |
| 1.175 | CH₂N(SO₂CH₃)CH₂-cyclopropyl | CF₃ | |
| 1.176 | CH₂N(SO₂CH₃)-phenyl | CF₃ | |
| 1.177 | CH₂N(SO₂CH₃)-benzyl | CF₃ | |
| 1.178 | CH₂N(SO₂CH₃)CH₂CH═CH₂ | CF₃ | |
| 1.179 | CH₂N(SO₂CH₃)CH₂C≡CH | CF₃ | |
| 1.180 | CH₂N(CH₃)C(O)H | CF₃ | |
| 1.181 | CH₂N(CH₃)C(O)CH₃ | CF₃ | |
| 1.182 | CH₂N(CH₃)C(O)CH₂CH₃ | CF₃ | |
| 1.183 | CH₂N(CH₃)C(O)-phenyl | CF₃ | |
| 1.184 | CH₂N(CH₃)C(O)-benzyl | CF₃ | |
| 1.185 | CH₂N(CH₂CH₃)C(O)CH₃ | CF₃ | |
| 1.186 | 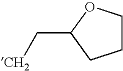 | CF₃ | |
| 1.187 | 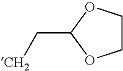 | CF₃ | |
| 1.188 | 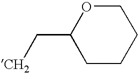 | CF₃ | |
| 1.189 | C(OCH₂CH₃)═CH₂ | CF₃ | m.p. 142-143° C. |
| 1.190 | CH₂C(O)CH₃ | CF₃ | |
| 1.191 | C(OCH₃)₂ | CF₃ | |
| 1.192 | 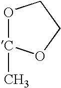 | CF₃ | waxy |
| 1.193 | CH₂C(O)CH₂OCH₃ | CF₃ | |
| 1.194 | CH₂C(O)CH₂OCH₂CH₂OCH₃ | CF₃ | |
| 1.195 | CH₂C(O)CH₂N(SO₂CH₃)CH₃ | CF₃ | |
| 1.196 | C(CH₂OCH₃)═CH₂ | CF₃ | |
| 1.197 | 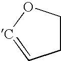 | CF₃ | |
| 1.198 | 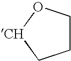 | CF₃ | |
| 1.199 | 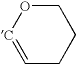 | CF₃ | crystalline solid |
| 1.200 | 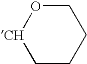 | CF₃ | |
| 1.201 | 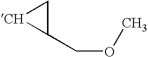 | CF₃ | cis and/or trans |
| 1.202 | 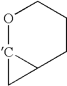 | CF₃ | |
| 1.203 | 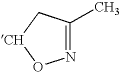 | CF₃ | amorphous solid |
| 1.204 | 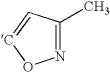 | CF₃ | amorphous solid |
| 1.205 | C(O)CH₃ | CF₃ | |
| 1.206 | C(O)CH₂OCH₃ | CF₃ | |
| 1.207 | C(O)CH₂OCH₂CH₂OCH₃ | CF₃ | |
| 1.208 | C(O)CH₂N(SO₂CH₃)CH₃ | CF₃ | |
| 1.209 | 4-CF₃-phenyl | CF₃ | resin |
| 1.210 | 4-MeO-phenyl | CF₃ | resin, [P15] |
| 1.211 | CH₃ | CF₃ | resin |
| 1.212 | C≡N | CF₃ | resin |
| 1.213 | 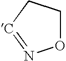 | CF₃ | |
| 1.214 | 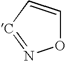 | CF₃ | |
| 1.215 | 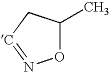 | CF₃ | |
| 1.216 | 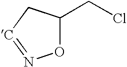 | CF₃ | |
| 1.217 | 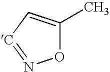 | CF₃ | |
| 1.218 | C(OCH₂CH₂CH₂CH₃)═CH₂ | CF₃ | |
| 1.219 | CH₂CH₂OCH₂CH₂CH₂CH₃ | CF₃ | |
| 1.220 | CH₂O-phenyl | CF₃ | |
| 1.221 | CH₂NHSO₂-phenyl | CF₃ | |
| 1.222 | CH₂N(SO₂CH₃)-cyclopropyl | CF₃ | |
| 1.223 | CH₂N(SO₂CH₂CH₃)-phenyl | CF₃ | |
| 1.224 | CH₂NHC(O)-cyclopropyl | CF₃ | |
| 1.225 | 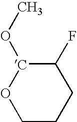 | CF₃ | |

TABLE 2
Compounds of formula IB

| | |
|---|---|
| | (IB) |
| 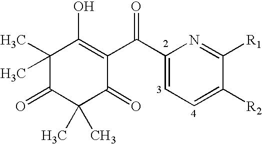 | |

| Ex. No. | R₁ | R₂ | Physical properties |
|---|---|---|---|
| 2.001 | OCH₂OCH₃ | CF₃ | |
| 2.002 | OCH₂OCH₂CH₃ | CF₃ | |
| 2.003 | OCH₂CH₂OCH₃ | CF₃ | resin |
| 2.004 | OCH₂CH₂OCH₂CH₃ | CF₃ | |
| 2.005 | OCH₂CH₂CH₂OCH₃ | CF₃ | |
| 2.006 | OCH(CH₃)CH₂OCH₃ | CF₃ | |
| 2.007 | OCH₂CH₂OCH₂CH═CH₂ | CF₃ | |
| 2.008 | OCH₂CH₂OCH₂C≡CH | CF₃ | |
| 2.009 | OCH₂CH₂O-benzyl | CF₃ | |
| 2.010 | OCH₂CH₂ON═C(CH₃)₂ | CF₃ | resin |
| 2.011 | OCH₂CH₂OCH₂CH₂OCH₃ | CF₃ | |
| 2.012 | OCH₂CH(OCH₃)₂ | CF₃ | |
| 2.013 | OCH₂CH(OCH₂CH₃)₂ | CF₃ | |
| 2.014 | 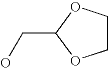 | CF₃ | |
| 2.015 | 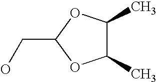 | CF₃ | |
| 2.016 | 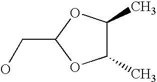 | CF₃ | |
| 2.017 | 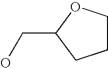 | CF₃ | |
| 2.018 | 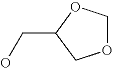 | CF₃ | |
| 2.019 | 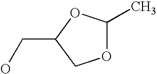 | CF₃ | |
| 2.020 | 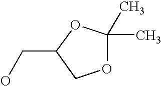 | CF₃ | |
| 2.021 | 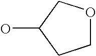 | CF₃ | resin |
| 2.022 | O-benzyl | CF₃ | resin |
| 2.023 | OCH₂CH₂SCH₃ | CF₃ | |
| 2.024 | OCH₂CH₂SCH₂CH₃ | CF₃ | |
| 2.025 | OCH₂CH₂S(O)₂CH₃ | CF₃ | |
| 2.026 | OCH₂CH₂S(O)₂CH₂CH₃ | CF₃ | |
| 2.027 | SCH₂CH₂OCH₃ | CF₃ | |
| 2.028 | SCH₂CH₂OCH₂CH₃ | CF₃ | |
| 2.029 | OCH₂CH₂OC(O)CH₃ | CF₃ | |
| 2.030 | OCH₂CH₂OC(O)-phenyl | CF₃ | |
| 2.031 | OCH₂CH₂OC(O)OCH₂CH₃ | CF₃ | |
| 2.032 | OCH₂CH₂OC(O)NHCH₂CH₃ | CF₃ | |
| 2.033 | OCH₂CH₂NH₂ | CF₃ | |
| 2.034 | OCH₂CH₂NHC(O)CH₃ | CF₃ | |
| 2.035 | OCH₂CH₂NHC(O)CH₂CH₃ | CF₃ | |
| 2.036 | OCH₂CH₂NHC(O)CH(CH₃)₂ | CF₃ | |
| 2.037 | OCH₂CH₂NHC(O)-cyclopropyl | CF₃ | |
| 2.038 | OCH₂CH₂NHC(O)C(CH₃)₃ | CF₃ | |
| 2.039 | OCH₂CH₂NHC(O)-phenyl | CF₃ | |
| 2.040 | OCH₂CH₂NHC(O)OCH₃ | CF₃ | |
| 2.041 | OCH₂CH₂NHC(O)OCH₂CH₃ | CF₃ | |
| 2.042 | OCH₂CH₂NHC(O)NHCH₃ | CF₃ | |
| 2.043 | OCH₂CH₂NHC(O)NHCH₂CH₃ | CF₃ | |
| 2.044 | OCH₂CH₂NHC(O)N(CH₃)₂ | CF₃ | |
| 2.045 | OCH₂CH₂NHC(O)N(CH₂CH₃)₂ | CF₃ | |
| 2.046 | NHCH₃ | CF₃ | |
| 2.047 | NHCH₂CH₃ | CF₃ | |
| 2.048 | NHCH₂CH₂CH₃ | CF₃ | |
| 2.049 | NHCH₂CH₂CH₂CH₃ | CF₃ | |
| 2.050 | NHCH(CH₃)₂ | CF₃ | |
| 2.051 | NHC(CH₃)₃ | CF₃ | |
| 2.052 | NHCH₂-cyclopropyl | CF₃ | |
| 2.053 | NH-phenyl | CF₃ | |
| 2.054 | NH-benzyl | CF₃ | |
| 2.055 | NH—CH₂CH═CH₂ | CF₃ | |
| 2.056 | NHCH₂C≡CH | CF₃ | |
| 2.057 | N(CH₂CH═CH₂)₂ | CF₃ | |
| 2.058 | N(CH₂C≡CH)₂ | CF₃ | |
| 2.059 | N(CH₃)₂ | CF₃ | m.p.: 95-96° C. |
| 2.060 | N(CH₂CH₃)₂ | CF₃ | m.p.: 85-86° C. |
| 2.061 | N(CH₂CH₂CH₃)₂ | CF₃ | |
| 2.062 | N(CH₂CH₂CH₂CH₃)₂ | CF₃ | |
| 2.063 | NHCH₂CH₂OH | CF₃ | |
| 2.064 | NHCH₂CH₂OCH₃ | CF₃ | resin |
| 2.065 | NHCH(CH₃)CH₃OCH₃ | CF₃ | |
| 2.066 | NHCH₂CH(OCH₃)₂ | CF₃ | |
| 2.067 | NHCH₂CH(OCH₂CH₃)₂ | CF₃ | |
| 2.068 | 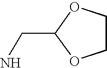 | CF₃ | |
| 2.069 | NHCH₂C(O)OCH₃ | CF₃ | |
| 2.070 | NHCH(CH₃)C(O)OCH₃ | CF₃ | |
| 2.071 | NHCH₂C(O)OCH₂CH₃ | CF₃ | |
| 2.072 | NHCH(CH₃)C(O)OCH₂CH₃ | CF₃ | |
| 2.073 | 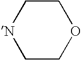 | CF₃ | m.p.: 123-124° C. |
| 2.074 | 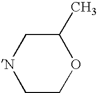 | CF₃ | |
| 2.075 | 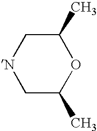 | CF₃ | m.p.: 134-135° C. |
| 2.076 | 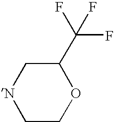 | CF₃ | |
| 2.077 | 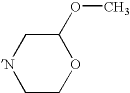 | CF₃ | |
| 2.078 | 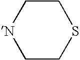 | CF₃ | m.p.: 120-121° C. |
| 2.079 | 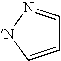 | CF₃ | |
| 2.080 | 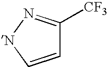 | CF₃ | m.p.: 99-100° C. |
| 2.081 | 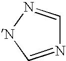 | CF₃ | |
| 2.082 | 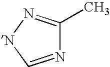 | CF₃ | |
| 2.083 | 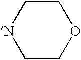 | CHF₂ | |
| 2.084 | 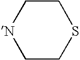 | CHF₂ | |
| 2.085 | 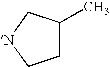 | CHF₂ | |
| 2.086 | 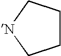 | CHF₂ | |
| 2.087 | N(CH₃)C(O)H | CF₃ | |
| 2.088 | N(CH₃)C(O)CH₃ | CF₃ | m.p.: 150-151° C. |
| 2.089 | N(CH₃)C(O)CH₂CH₃ | CF₃ | m.p.: 117-118° C. |
| 2.090 | N(CH₃)C(O)-phenyl | CF₃ | resin |
| 2.091 | N(CH₃)C(O)-benzyl | CF₃ | m.p.: 107-108° C. |
| 2.092 | N(CH₂CH₃)C(O)CH₃ | CF₃ | |
| 2.093 | OH | CF₃ | m.p.: 203-204° C. |
| 2.094 | OCH₃ | CF₃ | m.p.: 120-121° C. |
| 2.095 | OCH₂CH₃ | CF₃ | m.p.: 117-118° C. |
| 2.096 | CH₂OH | CF₃ | |
| 2.097 | CH₂Cl | CF₃ | |
| 2.098 | CH₂Br | CF₃ | |
| 2.099 | CH₂OSO₂CH₃ | CF₃ | |
| 2.100 | CH₂OC(O)CH₃ | CF₃ | |
| 2.101 | CH₂OC(O)C(CH₃)₃ | CF₃ | |
| 2.102 | CH₂OC(O)phenyl | CF₃ | |
| 2.103 | CH₂OC(O)OCH₂CH₃ | CF₃ | |
| 2.104 | CH₂OCH₃ | CF₃ | |
| 2.105 | CH₂OCH₂CH₃ | CF₃ | |
| 2.106 | CH₂CH₂OCH₃ | CF₃ | |
| 2.107 | CH₂CH₂OCH₂CH₃ | CF₃ | |
| 2.108 | CH₂CH₂CH₂OCH₃ | CF₃ | |
| 2.109 | CH(CH₃)CH₂OCH₃ | CF₃ | |
| 2.110 | CH₂OCH₂CH═CH₂ | CF₃ | |
| 2.111 | CH₂OCH₂C≡CH | CF₃ | |
| 2.112 | CH₂OCH₂C≡CCH₃ | CF₃ | |
| 2.113 | CH₂OCH₂CH₂C≡CH | CF₃ | |
| 2.114 | CH₂OCH₂CH₂C≡CCH₃ | CF₃ | |
| 2.115 | CH₂O-benzyl | CF₃ | |
| 2.116 | CH₂OCH₂CF₃ | CF₃ | |
| 2.117 | CH₂OCH₂CH₂F | CF₃ | |
| 2.118 | CH₂OCH₂CH₂Cl | CF₃ | |
| 2.119 | CH₂OCH₂CH₂Br | CF₃ | |
| 2.120 | CH₂OCH₂CH₂C≡N | CF₃ | |
| 2.121 | CH₂OCH₂C≡N | CF₃ | |
| 2.122 | CH₂OCH₂OCH₃ | CF₃ | |
| 2.123 | CH₂OCH₂OCH₂CH₃ | CF₃ | |
| 2.124 | CH₂OCH₂CH₂OH | CF₃ | |
| 2.125 | CH₂OCH₂CH₂OCH₃ | CF₃ | |
| 2.126 | CH₂OCH₂CH₂OCH₂CH₃ | CF₃ | |
| 2.127 | CH₂OCH₂CH₂CH₂OCH₃ | CF₃ | |
| 2.128 | CH₂OCH(CH₃)CH₂OCH₃ | CF₃ | |
| 2.129 | CH₂OCH₂CH₂OCH₂CH═CH₂ | CF₃ | |
| 2.130 | CH₂OCH₂CH₂OCH₂C≡CH | CF₃ | |
| 2.131 | CH₂OCH₂CH₂O-benzyl | CF₃ | |
| 2.132 | CH₂OCH₂CH₂ON═C(CH₃)₂ | CF₃ | |
| 2.133 | CH₂OCH₂CH₃OCH₂CH₂OCH₃ | CF₃ | |
| 2.134 | CH₂OCH₂CH(OCH₃)₂ | CF₃ | |
| 2.135 | CH₂OCH₂CH(OCH₂CH₃)₂ | CF₃ | |
| 2.136 | 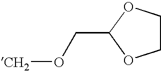 | CF₃ | |
| 2.137 | 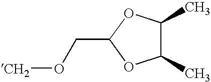 | CF₃ | |
| 2.138 | 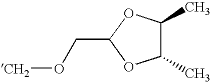 | CF₃ | |
| 2.139 | 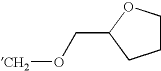 | CF₃ | |
| 2.140 | 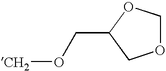 | CF₃ | |
| 2.141 | 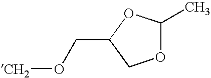 | CF₃ | |
| 2.142 | 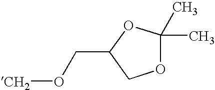 | CF₃ | |
| 2.143 | 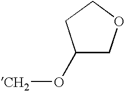 | CF₃ | |
| 2.144 | 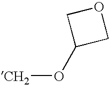 | CF₃ | |
| 2.145 | 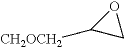 | CF₃ | |
| 2.146 | CH₂OCH₂CH₂SCH₃ | CF₃ | |
| 2.147 | CH₂OCH₂CH₂SCH₂CH₃ | CF₃ | |
| 2.148 | CH₂OCH₂CH₂S(O)₂CH₃ | CF₃ | |
| 2.149 | CH₂OCH₂CH₂S(O)₂CH₂CH₃ | CF₃ | |
| 2.150 | CH₂SCH₂CH₂OCH₃ | CF₃ | |
| 2.151 | CH₂SCH₂CH₂OCH₂CH₃ | CF₃ | |
| 2.152 | CH₂OCH₂CH₂OC(O)CH₃ | CF₃ | |
| 2.153 | CH₂OCH₂CH₂OC(O)-phenyl | CF₃ | |
| 2.154 | CH₂OCH₂CH₂OC(O)OCH₂CH₃ | CF₃ | |
| 2.155 | CH₂OCH₂CH₂OC(O)NHCH₂CH₃ | CF₃ | |
| 2.156 | CH₂OCH₂CH₂NH₂ | CF₃ | |
| 2.157 | CH₂OCH₂CH₂NHC(O)CH₃ | CF₃ | |
| 2.158 | CH₂OCH₂CH₂NHC(O)CH₂CH₃ | CF₃ | |
| 2.159 | CH₂OCH₂CH₂NHC(O)CH(CH₃)₂ | CF₃ | |
| 2.160 | CH₂OCH₂CH₂NHC(O)-cyclopropyl | CF₃ | |
| 2.161 | CH₂OCH₂CH₂NHC(O)C(CH₃)₃ | CF₃ | |
| 2.162 | CH₂OCH₂CH₂NHC(O)-phenyl | CF₃ | |
| 2.163 | CH₂OCH₂CH₂NHC(O)OCH₃ | CF₃ | |
| 2.164 | CH₂OCH₂CH₂NHC(O)OCH₂CH₃ | CF₃ | |
| 2.165 | CH₂OCH₂CH₂NHC(O)NHCH₃ | CF₃ | |
| 2.166 | CH₂OCH₂CH₂NHC(O)NHCH₂CH₃ | CF₃ | |
| 2.167 | CH₂OCH₂CH₂NHC(O)N(CH₃)₂ | CF₃ | |
| 2.168 | CH₂OCH₂CH₂NHC(O)N(CH₂CH₃)₂ | CF₃ | |
| 2.169 | CH₂N(SO₂CH₃)CH₃ | CF₃ | |
| 2.170 | CH₂N(SO₂CH₃)CH₂CH₃ | CF₃ | |
| 2.171 | CH₂N(SO₂CH₂CH₃)CH₂CH₃ | CF₃ | |
| 2.172 | CH₂N(SO₂CH₃)CH₂CF₃ | CF₃ | |
| 2.173 | CH₂N(SO₂CH₃)CH₂CHOCH₃ | CF₃ | |
| 2.174 | CH₂N(SO₂CH₃)CH₂-cyclopropyl | CF₃ | |
| 2.175 | CH₂N(SO₂CH₃)-phenyl | CF₃ | |
| 2.176 | CH₂N(SO₂CH₃)-benzyl | CF₃ | |
| 2.177 | CH₂N(SO₂CH₃)CH₂CH═CH₂ | CF₃ | |
| 2.178 | CH₂N(SO₂CH₃)CH₂C≡CH | CF₃ | |
| 2.179 | CH₂N(CH₃)C(O)H | CF₃ | |
| 2.180 | CH₂N(CH₃)C(O)CH₃ | CF₃ | |
| 2.181 | CH₂N(CH₃)C(O)CH₂CH₃ | CF₃ | |
| 2.182 | CH₂N(CH₃)C(O)-phenyl | CF₃ | |
| 2.183 | CH₂N(CH₃)C(O)-benzyl | CF₃ | |
| 2.184 | CH₂N(CH₂CH₃)C(O)CH₃ | CF₃ | |
| 2.185 | 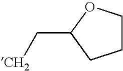 | CF₃ | |
| 2.186 | 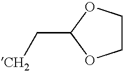 | CF₃ | |
| 2.187 | 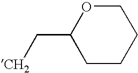 | CF₃ | |
| 2.188 | O(OCH₂CH₃)═CH₂ | CF₃ | waxy |
| 2.189 | CH₂C(O)CH₃ | CF₃ | |
| 2.190 | C(OCH₃)₂ | CF₃ | |
| 2.191 | 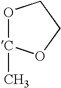 | CF₃ | m.p.: 108-110° C. |
| 2.192 | CH₂C(O)CH₂OCH₃ | CF₃ | |
| 2.193 | CH₂C(O)CH₂OCH₂CH₂OCH₃ | CF₃ | |
| 2.194 | CH₂C(O)CH₂N(SO₂CH₃)CH₃ | CF₃ | |
| 2.195 | C(CH₂OCH₃)═CH₂ | CF₃ | |
| 2.196 | 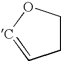 | CF₃ | |
| 2.197 | 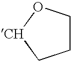 | CF₃ | |
| 2.198 | 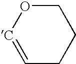 | CF₃ | crystalline solid |
| 2.199 | 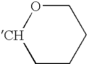 | CF₃ | |
| 2.200 | 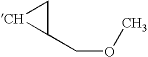 | CF₃ | |
| 2.201 | 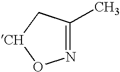 | CF₃ | amorphous solid |
| 2.202 | 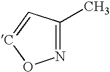 | CF₃ | amorphous solid |
| 2.203 | CH₃ | CF₃ | |
| 2.204 | C(O)CH₃ | CF₃ | |
| 2.205 | C(O)CH₂OCH₃ | CF₃ | |
| 2.206 | C(O)CH₂OCH₂CH₂OCH₃ | CF₃ | |
| 2.207 | C(O)CH₂N(SO₂CH₃)CH₃ | CF₃ | |
| 2.208 | C≡N | CF₃ | resin |
| 2.209 | CH₃ | C≡N | resin |
| 2.210 | 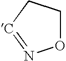 | CF₃ | |
| 2.211 | 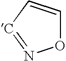 | CF₃ | |
| 2.212 | 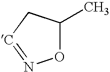 | CF₃ | |
| 2.213 | 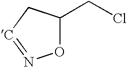 | CF₃ | |
| 2.214 | 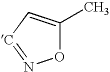 | CF₃ | |
| 2.215 | C(OCH₂CH₂CH₂CH₃)═CH₂ | CF₃ | |
| 2.216 | CH₂CH₂OCH₂CH₂CH₂CH₃ | CF₃ | |
| 2.217 | CH₂O-phenyl | CF₃ | |
| 2.218 | CH₂NHSO₂-phenyl | CF₃ | |
| 2.219 | CH₂N(SO₂CH₃)-cyclopropyl | CF₃ | |
| 2.220 | CH₂N(SO₂CH₂CH₃)-phenyl | CF₃ | |
| 2.221 | CH₂NHC(O)-cyclopropyl | CF₃ | |
| 2.222 | 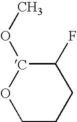 | CF₃ | isomer A (cis or trans), crystalline solid |
| 2.223 | 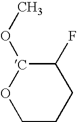 | CF₃ | isomer B (cis or trans), crystalline solid |

TABLE 3
Compounds of formula IC

| | |
|---|---|
| | (IC) |
| 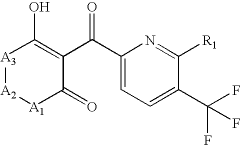 | |

| Ex. | | | | | Physical |
|---|---|---|---|---|---|
| No. | A₁ | A₂ | A₃ | R₁ | properties |
| 3.001 | CH₂ | CH₂ | CH(CH₃) | OCH₂OCH₃ | |
| 3.002 | CH₂ | CH₂ | CH(CH₃) | OCH₂OCH₂CH₃ | |
| 3.003 | CH₂ | CH₂ | CH(CH₃) | OCH₂CH₂OCH₃ | |
| 3.004 | CH₂ | CH₂ | CH(CH₃) | OCH₂CH₂OCH₂CH₃ | |
| 3.005 | CH₂ | CH₂ | CH(CH₃) | OCH₂CH₂CH₂OCH₃ | |
| 3.006 | CH₂ | CH₂ | CH(CH₃) | OCH(CH₃)CH₂OCH₃ | |
| 3.007 | CH₂ | CH₂ | CH(CH₃) | OCH₂CH₂OCH₂CH═CH₂ | |
| 3.008 | CH₂ | CH₂ | CH(CH₃) | OCH₂CH₂OCH₂C≡CH | |
| 3.009 | CH₂ | CH₂ | CH(CH₃) | OCH₂CH₂O-benzyl | |
| 3.010 | CH₂ | CH₂ | CH(CH₃) | OCH₂CH₂ON═C(CH₃)₂ | |
| 3.011 | CH₂ | CH₂ | CH(CH₃) | OCH₂CH₂OCH₂CH₂OCH₃ | |
| 3.012 | CH₂ | CH₂ | CH(CH₃) | OCH₂CH(OCH₃)₂ | |
| 3.013 | CH₂ | CH₂ | CH(CH₃) | OCH₂OH(OCH₂CH₃)₂ | |
| 3.014 | CH₂ | CH₂ | CH(CH₃) | 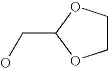 | |
| 3.015 | CH₂ | CH₂ | CH(CH₃) | 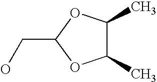 | |
| 3.016 | CH₂ | CH₂ | CH(CH₃) | 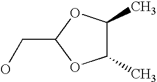 | |
| 3.017 | CH₂ | CH₂ | CH(CH₃) | 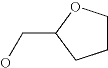 | |
| 3.018 | CH₂ | CH₂ | CH(CH₃) | 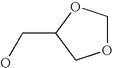 | |
| 3.019 | CH₂ | CH₂ | CH(CH₃) | 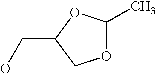 | |
| 3.020 | CH₂ | CH₂ | CH(CH₃) | 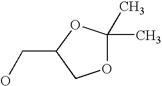 | |
| 3.021 | CH₂ | CH₂ | CH(CH₃) | 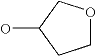 | |
| 3.022 | CH₂ | CH₂ | CH(CH₃) | O-benzyl | |
| 3.023 | CH(CH₃) | CH₂ | CH₂ | OCH₂CH₂SCH₃ | |
| 3.024 | CH₂ | CH₂ | CH(CH₃) | OCH₂CH₂SCH₂CH₃ | |
| 3.025 | CH₂ | CH₂ | CH(CH₃) | OCH₂CH₂S(O)₂CH₃ | |
| 3.026 | CH₂ | CH₂ | CH(CH₃) | OCH₂CH₂S(O)₂CH₂CH₃ | |
| 3.027 | CH₂ | CH₂ | CH(CH₃) | SCH₂CH₂OCH₃ | |
| 3.028 | CH₂ | CH₂ | CH(CH₃) | SCH₂CH₂OCH₂CH₃ | |
| 3.029 | CH₂ | CH₂ | CH(CH₃) | OCH₂CH₂OC(O)CH₃ | |
| 3.030 | CH₂ | CH₂ | CH(CH₃) | OCH₂CH₂OC(O)-phenyl | |
| 3.031 | CH₂ | CH₂ | CH(CH₃) | OCH₂CH₂OC(O)OCH₂CH₃ | |
| 3.032 | CH₂ | CH₂ | CH(CH₃) | OCH₂CH₂OC(O)NHCH₂CH₃ | |
| 3.033 | CH₂ | CH₂ | CH(CH₃) | OCH₂CH₂NH₂ | |
| 3.034 | CH₂ | CH₂ | CH(CH₃) | OCH₂CH₂NHC(O)CH₃ | |
| 3.035 | CH₂ | CH₂ | CH(CH₃) | OCH₂CH₂NHC(O)CH₂CH₃ | |
| 3.036 | CH₂ | CH₂ | CH(CH₃) | OCH₂CH₂NHC(O)CH(CH₃)₂ | |
| 3.037 | CH₂ | CH₂ | CH(CH₃) | OCH₂CH₂NHC(O)-cyclopropyl | |
| 3.038 | CH₂ | CH₂ | CH(CH₃) | OCH₂CH₂NHC(O)C(CH₃)₃ | |
| 3.039 | CH₂ | CH₂ | CH(CH₃) | OCH₂CH₂NHC(O)-phenyl | |
| 3.040 | CH₂ | CH₂ | CH(CH₃) | OCH₂CH₂NHC(O)OCH₃ | |
| 3.041 | CH₂ | CH₂ | CH(CH₃) | OCH₂CH₂NHC(O)OCH₂CH₃ | |
| 3.042 | CH₂ | CH₂ | CH(CH₃) | OCH₂CH₂NHC(O)NHCH₃ | |
| 3.043 | CH₂ | CH₂ | CH(CH₃) | OCH₂CH₂NHC(O)NHCH₂CH₃ | |
| 3.044 | CH₂ | CH₂ | CH(CH₃) | OCH₂CH₂NHC(O)N(CH₃)₂ | |
| 3.045 | CH₂ | CH₂ | CH(CH₃) | OCH₂CH₂NHC(O)N(CH₂CH₃)₂ | |
| 3.046 | CH₂ | CH₂ | CH(CH₃) | NHCH₃ | |
| 3.047 | CH₂ | CH₂ | CH(CH₃) | NHCH₂CH₃ | |
| 3.048 | CH₂ | CH₂ | CH(CH₃) | NHCH₂CH₂CH₃ | |
| 3.049 | CH₂ | CH₂ | CH(CH₃) | NHCH₂CH₂CH₂CH₃ | |
| 3.050 | CH₂ | CH₂ | CH(CH₃) | NHCH(CH₃)₂ | |
| 3.051 | CH₂ | CH₂ | CH(CH₃) | NHC(CH₃)₃ | |
| 3.052 | CH₂ | CH₂ | CH(CH₃) | NHCH₂-cyclopropyl | |
| 3.053 | CH₂ | CH₂ | CH(CH₃) | NH-phenyl | |
| 3.054 | CH₂ | CH₂ | CH(CH₃) | NH-benzyl | |
| 3.055 | CH₂ | CH₂ | CH(CH₃) | N(CH₂CH₃)₂ | |
| 3.056 | CH₂ | CH₂ | CH(CH₃) | N(CH₂CH₂CH₃)₂ | |
| 3.057 | CH₂ | CH₂ | CH(CH₃) | N(CH₂CH₂CH₂CH₃)₂ | |
| 3.058 | CH₂ | CH₂ | CH(CH₃) | NHCH₂CH₂OH | |
| 3.059 | CH₂ | CH₂ | CH(CH₃) | NHCH₂CH₂OCH₃ | |
| 3.060 | CH₂ | CH₂ | CH(CH₃) | NHCH(CH₃)CH₃OCH₃ | |
| 3.061 | CH₂ | CH₂ | CH(CH₃) | NHCH₂CH(OCH₃)₂ | |
| 3.062 | CH₂ | CH₂ | CH(CH₃) | NHCH₂CH(OCH₂CH₃)₂ | |
| 3.063 | CH₂ | CH₂ | CH(CH₃) | 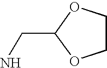 | |
| 3.064 | CH₂ | CH₂ | CH(CH₃) | NHCH₂C(O)CCH₃ | |
| 3.065 | CH₂ | CH₂ | CH(CH₃) | NHCH(CH₃)C(O)OCH₂ | |
| 3.066 | CH₂ | CH₂ | CH(CH₃) | NHCH₂C(O)OCH₂CH₃ | |
| 3.067 | CH₂ | CH₂ | CH(CH₃) | NHCH(CH₃)C(O)OCH₂CH₃ | |
| 3.068 | CH₂ | CH₂ | 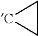 | 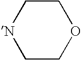 | resin |
| 3.069 | CH₂ | CH(CH₃) | CH₂ | 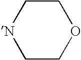 | m.p.: 80-81° C. |
| 3.070 | 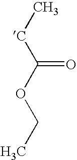 | CH₂ | CH₂ | 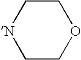 | resin |
| 3.071 | CH(CH₃) | CH₂ | CH₂ | 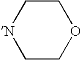 | resin |
| 3.072 | C(CH₃)₂ | CH₂ | CH₂ | 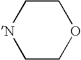 | resin |
| 3.073 | C(CH₃)₂ | CH(CH₃) | CH₂ | 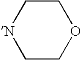 | resin |
| 3.074 | C(CH₃)₂ | CH₂ | CH(CH₃) | 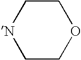 | resin |
| 3.075 | C(CH₃)₂ | CH₂ | C(CH₃)₂ | 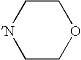 | resin |
| 3.076 | C(CH₃)₂ | O | C(CH₃)₂ | 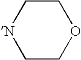 | resin |
| 3.077 | CH₂ | C(CH₃)₂ | CH₂ | 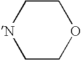 | m.p.: 121-122° C. |
| 3.078 | CH(CH₃) | CH₂ | CH₂ | 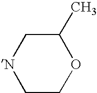 | |
| 3.079 | CH(CH₃) | CH₂ | CH₂ | 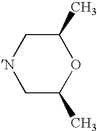 | |
| 3.080 | CH(CH₃) | CH₂ | CH₂ | 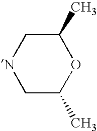 | |
| 3.081 | CH(CH₃) | CH₂ | CH₂ | 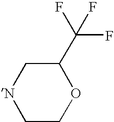 | |
| 3.082 | CH(CH₃) | CH₂ | CH₂ | 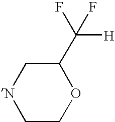 | |
| 3.083 | CH(CH₃) | CH₂ | CH₂ | 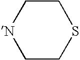 | |
| 3.084 | CH(CH₃) | CH₂ | CH₂ | 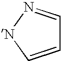 | |
| 3.085 | CH(CH₃) | CH₂ | CH₂ | 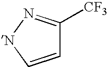 | |
| 3.086 | CH(CH₃) | CH₂ | CH₂ | 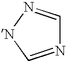 | |
| 3.087 | CH(CH₃) | CH₂ | CH₂ | 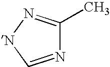 | |
| 3.088 | CH(CH₃) | CH₂ | CH₂ | 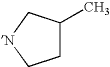 | |
| 3.089 | CH(CH₃) | CH₂ | CH₂ | 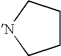 | |
| 3.090 | CH(CH₃) | CH₂ | CH₂ | N(CH₃)C(O)H | |
| 3.091 | CH(CH₃) | CH₂ | CH₂ | N(CH₃)C(O)CH₃ | |
| 3.092 | CH(CH₃) | CH₂ | CH₂ | N(CH₃)C(O)CH₂CH₃ | |
| 3.093 | CH(CH₃) | CH₂ | CH₂ | N(CH₃)C(O)-phenyl | |
| 3.094 | CH(CH₃) | CH₂ | CH₂ | N(CH₃)C(O)-benzyl | |
| 3.095 | CH(CH₃) | CH₂ | CH₂ | N(CH₂CH₃)C(O)CH₃ | |
| 3.096 | CH₂ | CH(CH₃) | CH₂ | CH₂OH | |
| 3.097 | CH₂ | CH(CH₃) | CH₂ | CH₂Cl | |
| 3.098 | CH₂ | CH(CH₃) | CH₂ | CH₂Br | |
| 3.099 | CH₂ | CH(CH₃) | CH₂ | CH₂OSO₂CH₃ | |
| 3.100 | CH₂ | CH(CH₃) | CH₂ | CH₂OC(O)CH₃ | |
| 3.101 | CH₂ | CH(CH₃) | CH₂ | CH₂OC(O)C(CH₃)₃ | |
| 3.102 | CH₂ | CH(CH₃) | CH₂ | CH₂OC(O)phenyl | |
| 3.103 | CH₂ | CH(CH₃) | CH₂ | CH₂OC(O)OCH₂CH₃ | |
| 3.104 | CH₂ | CH(CH₃) | CH₂ | CH₂OCH₃ | |
| 3.105 | CH₂ | CH(CH₃) | CH₂ | CH₂OCH₂CH₃ | |
| 3.106 | CH₂ | CH(CH₃) | CH₂ | CH₂CH₂OCH₃ | |
| 3.107 | CH₂ | CH(CH₃) | CH₂ | CH₂CH₂OCH₂CH₃ | |
| 3.108 | CH₂ | CH(CH₃) | CH₂ | CH₂CH₂CH₂OCH₃ | |
| 3.109 | CH₂ | CH(CH₃) | CH₂ | CH(CH₃)CH₂OCH₃ | |
| 3.110 | CH₂ | CH(CH₃) | CH₂ | CH₂OCH₂OH═CH₂ | |
| 3.111 | CH₂ | CH(CH₃) | CH₂ | CH₂OCH₂C≡CH | |
| 3.112 | CH₂ | CH(CH₃) | CH₂ | CH₂OCH₂C≡CCH₃ | |
| 3.113 | CH₂ | CH(CH₃) | CH₂ | CH₂OCH₂CH₂C≡CH | |
| 3.114 | CH₂ | CH(CH₃) | CH₂ | CH₂OCH₂CH₂C≡CCH₃ | |
| 3.115 | CH₂ | CH(CH₃) | CH₂ | CH₂O-benzyl | |
| 3.116 | CH₂ | CH(CH₃) | CH₂ | CH₂OCH₂CF₃ | |
| 3.117 | CH₂ | CH(CH₃) | CH₂ | CH₂OCH₂CH₂F | |
| 3.118 | CH₂ | CH(CH₃) | CH₂ | CH₂OCH₂CH₂Cl | |
| 3.119 | CH₂ | CH(CH₃) | CH₂ | CH₂OCH₂CH₂Br | |
| 3.120 | CH₂ | CH(CH₃) | CH₂ | CH₂OCH₂CH₂C≡N | |
| 3.121 | CH₂ | CH(CH₃) | CH₂ | CH₂OCH₂C≡N | |
| 3.122 | CH₂ | CH(CH₃) | CH₂ | CH₂OCH₂OCH₃ | |
| 3.123 | CH₂ | CH(CH₃) | CH₂ | CH₂OCH₂OCH₂CH₃ | |
| 3.124 | CH₂ | CH(CH₃) | CH₂ | CH₂OCH₂CH₂OH | |
| 3.125 | CH₂ | CH(CH₃) | CH₂ | CH₂OCH₂CH₂OCH₃ | |
| 3.126 | CH₂ | CH(CH₃) | CH₂ | CH₂OCH₂CH₂OCH₂CH₃ | |
| 3.127 | CH₂ | CH(CH₃) | CH₂ | CH₂OCH₂CH₂CH₂OCH₃ | |
| 3.128 | CH₂ | CH(CH₃) | CH₂ | CH₂OCH(CH₃)CH₂OCH₃ | |
| 3.129 | CH₂ | CH(CH₃) | CH₂ | CH₂OCH₂CH₂OCH₂CH═CH₂ | |
| 3.130 | CH₂ | CH(CH₃) | CH₂ | CH₂OCH₂CH₂OCH₂C≡CH | |
| 3.131 | CH₂ | CH(CH₃) | CH₂ | CH₂OCH₂CH₂O-benzyl | |
| 3.132 | CH₂ | CH(CH₃) | CH₂ | CH₂OCH₂CH₂ON═C(CH₃)₂ | |
| 3.133 | CH₂ | CH(CH₃) | CH₂ | CH₂OCH₂CH₂OCH₂CH₂OCH₃ | |
| 3.134 | CH₂ | CH(CH₃) | CH₂ | CH₂OCH₂CH(OCH₃)₂ | |
| 3.135 | CH₂ | CH(CH₃) | CH₂ | CH₂OCH₂CH(OCH₂CH₃)₂ | |
| 3.136 | CH₂ | CH(CH₃) | CH₂ | 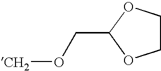 | |
| 3.137 | CH₂ | CH(CH₃) | CH₂ | 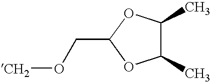 | |
| 3.138 | CH₂ | CH(CH₃) | CH₂ | 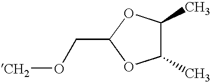 | |
| 3.139 | CH₂ | CH(CH₃) | CH₂ | 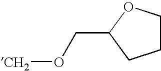 | |
| 3.140 | CH₂ | CH(CH₃) | CH₂ | 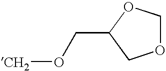 | |
| 3.141 | CH₂ | CH(CH₃) | CH₂ | 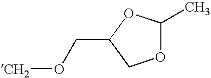 | |
| 3.142 | CH₂ | CH(CH₃) | CH₂ | 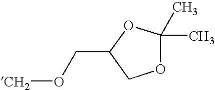 | |
| 3.143 | CH₂ | CH(CH₃) | CH₂ | 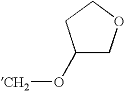 | |
| 3.144 | CH₂ | CH(CH₃) | CH₂ | 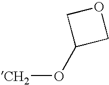 | |
| 3.145 | CH₂ | CH(CH₃) | CH₂ | 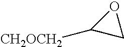 | |
| 3.146 | CH₂ | CH(CH₃) | CH₂ | CH₂OCH₂CH₂SCH₃ | |
| 3.147 | CH₂ | CH(CH₃) | CH₂ | CH₂OCH₂CH₂SCH₂CH₃ | |
| 3.148 | CH₂ | CH(CH₃) | CH₂ | CH₂OCH₂CH₂S(O)₂CH₃ | |
| 3.149 | CH₂ | CH(CH₃) | CH₂ | CH₂OCH₂CH₂S(O)₂CH₂CH₃ | |
| 3.150 | CH₂ | CH(CH₃) | CH₂ | CH₂SCH₂CH₂OCH₃ | |
| 3.151 | CH₂ | CH(CH₃) | CH₂ | CH₂SCH₂CH₂OCH₂CH₃ | |
| 3.152 | CH₂ | CH(CH₃) | CH₂ | CH₂OCH₂CH₂OC(O)CH₃ | |
| 3.153 | CH₂ | CH(CH₃) | CH₂ | CH₂OCH₂CH₂OC(O)-phenyl | |
| 3.154 | CH₂ | CH(CH₃) | CH₂ | CH₂OCH₂CH₂OC(O)OCH₂CH₃ | |
| 3.155 | CH₂ | CH(CH₃) | CH₂ | CH₂OCH₂CH₂OC(O)NHCH₂CH₃ | |
| 3.156 | CH₂ | CH(CH₃) | CH₂ | CH₂OCH₂CH₂NH₂ | |
| 3.157 | CH₂ | CH(CH₃) | CH₂ | CH₂OCH₂CH₂NHC(O)CH₃ | |
| 3.158 | CH₂ | CH(CH₃) | CH₂ | CH₂OCH₂CH₂NHC(O)CH₂CH₃ | |
| 3.159 | CH₂ | CH(CH₃) | CH₂ | CH₂OCH₂CH₂NHC(O)CH(CH₃)₂ | |
| 3.160 | CH₂ | CH(CH₃) | CH₂ | CH₂OCH₂CH₂NHC(O)-cyclopropyl | |
| 3.161 | CH₂ | CH(CH₃) | CH₂ | CH₂OCH₂CH₂NHC(O)C(CH₃)₃ | |
| 3.162 | CH₂ | CH(CH₃) | CH₂ | CH₂OCH₂CH₂NHC(O)-phenyl | |
| 3.163 | CH₂ | CH(CH₃) | CH₂ | CH₂OCH₂CH₂NHC(O)OCH₃ | |
| 3.164 | CH₂ | CH(CH₃) | CH₂ | CH₂OCH₂CH₂NHC(O)OCH₂CH₃ | |
| 3.165 | CH₂ | CH(CH₃) | CH₂ | CH₂OCH₂CH₂NHC(O)NHCH₃ | |
| 3.166 | CH₂ | CH(CH₃) | CH₂ | CH₂OCH₂CH₂NHC(O)NHCH₂CH₃ | |
| 3.167 | CH₂ | CH(CH₃) | CH₂ | CH₂OCH₂CH₂NHC(O)N(CH₃)₂ | |
| 3.168 | CH₂ | CH(CH₃) | CH₂ | CH₂OCH₂CH₂NHC(O)N(CH₂CH₃)₂ | |
| 3.169 | CH₂ | CH(CH₃) | CH₂ | CH₂N(SO₂CH₃)CH₃ | |
| 3.170 | CH₂ | CH(CH₃) | CH₂ | CH₂N(SO₂CH₃)CH₂CH₃ | |
| 3.171 | CH₂ | CH(CH₃) | CH₂ | CH₂N(SO₂CH₂CH₃)CH₂CH₃ | |
| 3.172 | CH₂ | CH(CH₃) | CH₂ | CH₂N(SO₂CH₃)CH₂CF₃ | |
| 3.173 | CH₂ | CH(CH₃) | CH₂ | CH₂N(SO₂CH₃)CH₂CHOCH₃ | |
| 3.174 | CH₂ | CH(CH₃) | CH₂ | CH₂N(SO₂CH₃)CH₂-cyclopropyl | |
| 3.175 | CH₂ | CH(CH₃) | CH₂ | CH₂N(SO₂CH₃)-phenyl | |
| 3.176 | CH₂ | CH(CH₃) | CH₂ | CH₂N(SO₂CH₃)-benzyl | |
| 3.177 | CH₂ | CH(CH₃) | CH₂ | CH₂N(SO₂CH₃)CH₂CH═CH₂ | |
| 3.178 | CH₂ | CH(CH₃) | CH₂ | CH₂N(SO₂CH₃)CH₂C≡CH | |
| 3.179 | CH₂ | CH(CH₃) | CH₂ | CH₂N(CH₃)C(O)H | |
| 3.180 | CH₂ | CH(CH₃) | CH₂ | CH₂N(CH₃)C(O)CH₃ | |
| 3.181 | CH₂ | CH(CH₃) | CH₂ | CH₂N(CH₃)C(O)CH₂CH₃ | |
| 3.182 | CH₂ | CH(CH₃) | CH₂ | CH₂N(CH₃)C(O)-phenyl | |
| 3.183 | CH₂ | CH(CH₃) | CH₂ | CH₂N(CH₃)C(O)-benzyl | |
| 3.184 | CH₂ | CH(CH₃) | CH₂ | CH₂N(CH₂CH₃)C(O)CH₃ | |
| 3.185 | CH₂ | CH(CH₃) | CH₂ | 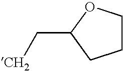 | |
| 3.186 | CH₂ | CH(CH₃) | CH₂ | 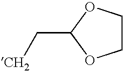 | |
| 3.187 | CH₂ | CH(CH₃) | CH₂ | 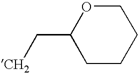 | |
| 3.188 | CH₂ | CH(CH₃) | CH₂ | C(OCH₂CH₃)═CH₂ | |
| 3.189 | CH₂ | CH(CH₃) | CH₂ | CH₂C(O)CH₃ | |
| 3.190 | CH₂ | CH(CH₃) | CH₂ | C(OCH₃)₂ | |
| 3.191 | CH₂ | CH(CH₃) | CH₂ | 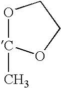 | |
| 3.192 | CH₂ | CH(CH₃) | CH₂ | CH₂C(O)CH₂OCH₃ | |
| 3.193 | CH₂ | CH(CH₃) | CH₂ | CH₂C(O)CH₂OCH₂CH₂OCH₃ | |
| 3.194 | CH₂ | CH(CH₃) | CH₂ | CH₂C(O)CH₂N(SO₂CH₃)CH₃ | |
| 3.195 | CH₂ | CH(CH₃) | CH₂ | C(CH₂OCH₃)═CH₂ | |
| 3.196 | CH₂ | CH(CH₃) | CH₂ | 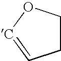 | |
| 3.197 | CH₂ | CH(CH₃) | CH₂ | 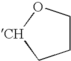 | |
| 3.198 | CH₂ | CH(CH₃) | CH₂ | 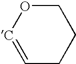 | |
| 3.199 | CH₂ | CH(CH₃) | CH₂ | 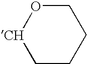 | |
| 3.200 | CH₂ | CH(CH₃) | CH₂ | 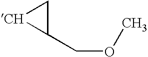 | |
| 3.201 | CH₂ | CH(CH₃) | CH₂ | Cl | resin |
| 3.202 | CH₂ | CH(CH₃) | CH₂ | CH₃ | resin |
| 3.203 | CH₂ | CH(CH₃) | CH₂ | OCH₂CH₃ | resin |
| 3.204 | CH₂ | CH₂ | C(CH₃)₂ | CH₃ | resin |
| 3.205 | CH₂ | CH₂ | C(CH₃)₂ | OCH₂CH₃ | resin |
| 3.206 | CH₂ | CH(CH₃) | C(CH₃)₂ | CH₃ | resin |
| 3.207 | CH₂ | CH(CH₃) | C(CH₃)₂ | OCH₂CH₃ | resin |
| 3.208 | CH(CH₃) | CH₂ | C(CH₃)₂ | CH₃ | resin |
| 3.209 | CH(CH₃) | CH₂ | C(CH₃)₂ | OCH₂CH₃ | resin |
| 3.210 | CH₂ | CH₂ | 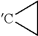 | CH₃ | resin |
| 3.211 | CH₂ | CH₂ | 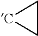 | OCH₂CH₃ | resin |
| 3.212 | CH₂ | CH(C≡N) | CH₂ | 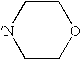 | resin |
| 3.213 | CH₂ | CH(CH₃) | CH₂ | 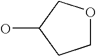 | resin |
| 3.214 | C(CH₃)₂ | CH₂ | CH₂ | 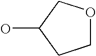 | resin |
| 3.215 | 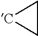 | CH₂ | CH₂ | 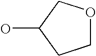 | resin |
| 3.216 | CH₂ | CH(CH₃) | CH₂ | 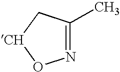 | |
| 3.217 | CH₂ | CH(CH₃) | CH₂ | 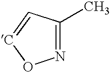 | |
| 3.218 | CH₂ | CH(CH₃) | CH₂ | 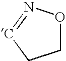 | |
| 3.219 | CH₂ | CH(CH₃) | CH₂ | 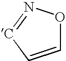 | |
| 3.220 | CH₂ | CH(CH₃) | CH₂ | C(OCH₂CH₂CH₂CH₃)═CH₂ | |
| 3.221 | CH₂ | CH(CH₃) | CH₂ | CH₂CH₂OCH₂CH₂CH₂CH₃ | |

TABLE Z1
Intermediates of formula II

| | |
|---|---|
| | (II) |
| 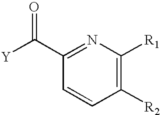 | |

| Ex. No. | Y | R₁ | R₂ | Physical properties |
|---|---|---|---|---|
| Z1.001 | OH | OCH₂OCH₃ | CF₃ | |
| Z1.002 | OH | OCH₂OCH₂CH₃ | CF₃ | |
| Z1.003 | OH | OCH₂CH₂OCH₃ | CF₃ | m.p.: 66-67° C. |
| Z1.004 | OH | OCH₂CH₂OCH₂CH₃ | CF₃ | |
| Z1.005 | OH | OCH₂CH₂CH₂OCH₃ | CF₃ | |
| Z1.006 | OH | OCH(CH₃)CH₂OCH₃ | CF₃ | |
| Z1.007 | OH | OCH₂CH₂OCH₂CH═CH₂ | CF₃ | |
| Z1.008 | OH | OCH₂CH₂OCH₂C≡CH | CF₃ | |
| Z1.009 | OH | OCH₂CH₂O-benzyl | CF₃ | |
| Z1.010 | OH | OCH₂CH₂ON═C(CH₃)₂ | CF₃ | m.p.: 106-107° C. |
| Z1.011 | OH | OCH₂CH₂OCH₂CH₂OCH₃ | CF₃ | |
| Z1.012 | OH | OCH₂CH(OCH₃)₂ | CF₃ | m.p.: 53-54° C. |
| Z1.013 | OH | OCH₂CH(OCH₂CH₃)₂ | CF₃ | |
| Z1.014 | OH | 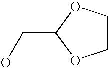 | CF₃ | amorphous |
| Z1.015 | OH | 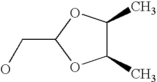 | CF₃ | |
| Z1.016 | OH | 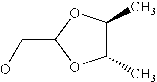 | CF₃ | |
| Z1.017 | OH | 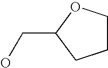 | CF₃ | |
| Z1.018 | OH | 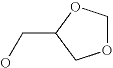 | CF₃ | |
| Z1.019 | OH | 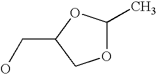 | CF₃ | |
| Z1.020 | OH | 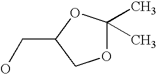 | CF₃ | |
| Z1.021 | OH | 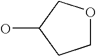 | CF₃ | m.p.: 124-125° C. |
| Z1.022 | OH | 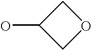 | CF₃ | waxy |
| Z1.023 | OH | O-benzyl | CF₃ | m.p.: 96-97° C. |
| Z1.024 | OH | OCH₂CH₂SCH₃ | CF₃ | |
| Z1.025 | OH | OCH₂CH₂SCH₂CH₃ | CF₃ | |
| Z1.026 | OH | OCH₂CH₂S(O)₂CH₃ | CF₃ | |
| Z1.027 | OH | OCH₂CH₂S(O)₂CH₂CH₃ | CF₃ | |
| Z1.028 | OH | SCH₂CH₂OCH₃ | CF₃ | |
| Z1.029 | OH | SCH₂CH₂OCH₂CH₃ | CF₃ | |
| Z1.030 | OH | OCH₂CH₂OC(O)CH₃ | CF₃ | |
| Z1.031 | OH | OCH₂CH₂OC(O)-phenyl | CF₃ | |
| Z1.032 | OH | OCH₂CH₂OC(O)OCH₂CH₃ | CF₃ | |
| Z1.033 | OH | OCH₂CH₂OC(O)NHCH₂CH₃ | CF₃ | |
| Z1.034 | OH | OCH₂CH₂NH₂ | CF₃ | |
| Z1.035 | OH | OCH₂CH₂NHC(O)CH₃ | CF₃ | |
| Z1.036 | OH | OCH₂CH₂NHC(O)CH₂CH₃ | CF₃ | |
| Z1.037 | OH | OCH₂CH₂NHC(O)CH(CH₃)₂ | CF₃ | |
| Z1.038 | OH | OCH₂CH₂NHC(O)-cyclopropyl | CF₃ | |
| Z1.039 | OH | OCH₂CH₂NHC(O)C(CH₃)₃ | CF₃ | |
| Z1.040 | OH | OCH₂CH₂NHC(O)-phenyl | CF₃ | |
| Z1.041 | OH | OCH₂CH₂NHC(O)OCH₃ | CF₃ | |
| Z1.042 | OH | OCH₂CH₂NHC(O)OCH₂CH₃ | CF₃ | m.p.: 118-119° C. |
| Z1.043 | OH | OCH₂CH₂NHC(O)NHCH₃ | CF₃ | |
| Z1.044 | OH | OCH₂CH₂NHC(O)NHCH₂CH₃ | CF₃ | |
| Z1.045 | OH | OCH₂CH₂NHC(O)N(CH₃)₂ | CF₃ | |
| Z1.046 | OH | OCH₂CH₂NHC(O)N(CH₂CH₃)₂ | CF₃ | |
| Z1.047 | OH | NHCH₃ | CF₃ | |
| Z1.048 | OH | NHCH₂CH₃ | CF₃ | |
| Z1.049 | OH | NHCH₂CH₂CH₃ | CF₃ | |
| Z1.050 | OH | NHCH₂CH₂CH₂CH₃ | CF₃ | |
| Z1.051 | OH | NHCH(CH₃)₂ | CF₃ | |
| Z1.052 | OH | NHC(CH₃)₃ | CF₃ | |
| Z1.053 | OH | NHCH₂-cyclopropyl | CF₃ | |
| Z1.054 | OH | NH-phenyl | CF₃ | |
| Z1.055 | OH | NH-benzyl | CF₃ | |
| Z1.056 | OH | NH—CH₂CH═CH₂ | CF₃ | |
| Z1.057 | OH | NHCH₂C≡CH | CF₃ | |
| Z1.058 | OH | N(CH₂CH═CH₂)₂ | CF₃ | |
| Z1.059 | OH | N(CH₂C≡CH)₂ | CF₃ | |
| Z1.060 | OH | N(CH₃)₂ | CF₃ | m.p.: 53-54° C. |
| Z1.061 | OH | N(CH₂CH₃)₂ | CF₃ | viscous oil |
| Z1.062 | OH | N(CH₂CH₂CH₃)₂ | CF₃ | |
| Z1.063 | OH | N(CH₂CH₂CH₂CH₃)₂ | CF₃ | |
| Z1.064 | OH | NHCH₂CH₂OH | CF₃ | |
| Z1.065 | OEt | NHCH₂CH₂OCH₃ | CF₃ | oil |
| Z1.066 | OH | NHCH(CH₃)CH₃OCH₃ | CF₃ | |
| Z1.067 | OH | NHCH₂CH(OCH₃)₂ | CF₃ | |
| Z1.068 | OH | NHCH₂CH(OCH₂CH₃)₂ | CF₃ | |
| Z1.069 | OH | 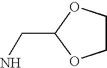 | CF₃ | |
| Z1.070 | OH | NHCH₂C(O)OCH₃ | CF₃ | |
| Z1.071 | OH | NHCH(CH₃)C(O)OCH₃ | CF₃ | |
| Z1.072 | OH | NHCH₂C(O)OCH₂CH₃ | CF₃ | |
| Z1.073 | OH | NHCH(CH₃)C(O)OCH₂CH₃ | CF₃ | |
| Z1.074 | OH | 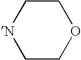 | CF₃ | m.p.: 115-116° C., [P5] |
| Z1.075 | OH | 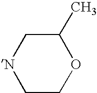 | CF₃ | |
| Z1.076 | OH | 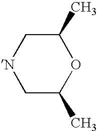 | CF₃ | m.p.: 127-128° C. |
| Z1.077 | OH | 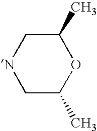 | CF₃ | |
| Z1.078 | OH | 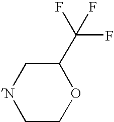 | CF₃ | |
| Z1.079 | OH | 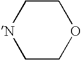 | CHF₂ | |
| Z1.080 | OH | 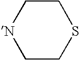 | CF₃ | m.p.: 103-104° C. |
| Z1.081 | OH | 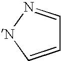 | CF₃ | |
| Z1.082 | OH | 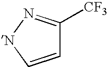 | CF₃ | amorphous |
| Z1.083 | OH | 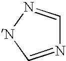 | CF₃ | |
| Z1.084 | OH | 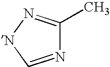 | CF₃ | |
| Z1.085 | OH | 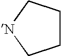 | CF₃ | |
| Z1.086 | OH | N(CH₃)C(O)H | CF₃ | |
| Z1.087 | OH | N(CH₃)C(O)CH₃ | CF₃ | m.p.: 164-165° C. |
| Z1.088 | OH | N(CH₃)C(O)CH₂CH₃ | CF₃ | m.p.: 76-77° C. |
| Z1.089 | OH | N(CH₃)C(O)-phenyl | CF₃ | m.p.: 137-138° C. |
| Z1.090 | OH | N(CH₃)C(O)-benzyl | CF₃ | m.p.: 154-156° C. |
| Z1.091 | OH | N(CH₂CH₃)C(O)CH₃ | CF₃ | |
| Z1.092 | OH | OH | CF₃ | m.p.: 220° C. |
| Z1.093 | OH | Cl | CF₃ | m.p.: 166-167° C. |
| Z1.094 | OH | OCH₃ | CF₃ | m.p.: 139-140° C. |
| Z1.095 | OH | OCH₂CH₃ | CF₃ | m.p.: 112-114° C. |
| Z1.096 | OH | 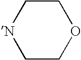 | OCH₂CF₃ | m.p.: 133-134° C. |
| Z1.097 | OEt | OH | CF₃ | m.p.: 141-141.5° C., [P2] |
| Z1.098 | OEt | SCH₃ | CF₃ | oil |
| Z1.099 | OEt | OCH₃ | CF₃ | m.p.: 71-73° C. |
| Z1.100 | OEt | OCH₂CF₃ | CF₃ | oil |
| Z1.101 | OEt | Cl | CF₃ | oil, [P3] |
| Z1.102 | OEt | CH₃ | CF₃ | oil, [P10] |
| Z1.103 | OEt | CH₂Br | CF₃ | oil, [P11] |
| Z1.104 | OEt | CHBr₂ | CF₃ | |
| Z1.105 | OEt | C(═O)H | CF₃ | |
| Z1.106 | OH | CH₂OH | CF₃ | |
| Z1.107 | OEt | CH₂Cl | CF₃ | |
| Z1.108 | OEt | CH₂OSO₂CH₃ | CF₃ | |
| Z1.109 | OH | CH₂OC(O)CH₃ | CF₃ | |
| Z1.110 | OH | CH₂OC(O)C(CH₃)₃ | CF₃ | |
| Z1.111 | OH | CH₂OC(O)phenyl | CF₃ | |
| Z1.112 | OH | CH₂OC(O)OCH₂CH₃ | CF₃ | |
| Z1.113 | OH | CH₂OCH₃ | CF₃ | |
| Z1.114 | OH | CH₂OCH₂CH₃ | CF₃ | |
| Z1.115 | OH | CH₂CH₂OCH₃ | CF₃ | |
| Z1.116 | OH | CH₂CH₂OCH₂CH₃ | CF₃ | |
| Z1.117 | OH | CH₂CH₂CH₂OCH₃ | CF₃ | |
| Z1.118 | OH | CH(CH₃)CH₂OCH₃ | CF₃ | |
| Z1.119 | OH | CH₂OCH₂CH═CH₂ | CF₃ | |
| Z1.120 | OH | CH₂OCH₂C≡CH | CF₃ | |
| Z1.121 | OH | CH₂OCH₂C≡CCH₃ | CF₃ | |
| Z1.122 | OH | CH₂OCH₂CH₂C≡CH | CF₃ | |
| Z1.123 | OH | CH₂OCH₂CH₂C≡CCH₃ | CF₃ | |
| Z1.124 | OH | CH₂O-benzyl | CF₃ | |
| Z1.125 | OH | CH₂OCH₂CF₃ | CF₃ | |
| Z1.126 | OH | CH₂OCH₂CH₂F | CF₃ | |
| Z1.127 | OH | CH₂OCH₂CH₂Cl | CF₃ | |
| Z1.128 | OH | CH₂OCH₂CH₂Br | CF₃ | |
| Z1.129 | OH | CH₂OCH₂CH₂C≡N | CF₃ | |
| Z1.130 | OH | CH₂OCH₂C≡N | CF₃ | |
| Z1.131 | OH | CH₂OCH₂OCH₃ | CF₃ | |
| Z1.132 | OH | CH₂OCH₂OCH₂CH₃ | CF₃ | |
| Z1.133 | OH | CH₂OCH₂CH₂OH | CF₃ | |
| Z1.134 | OH | CH₂OCH₂CH₂OCH₃ | CF₃ | waxy crystals |
| Z1.135 | OH | CH₂OCH₂CH₂OCH₂CH₃ | CF₃ | |
| Z1.136 | OH | CH₂OCH₂CH₂CH₂OCH₃ | CF₃ | |
| Z1.137 | OH | CH₂OCH(CH₃)CH₂OCH₃ | CF₃ | |
| Z1.138 | OH | CH₂OCH₂CH₂OCH₂CHCH₂ | CF₃ | |
| Z1.139 | OH | CH₂OCH₂CH₂OCH₂C≡CH | CF₃ | |
| Z1.140 | OH | CH₂OCH₂CH₂O-benzyl | CF₃ | |
| Z1.141 | OH | CH₂OCH₂CH₂ON═C(CH₃)₂ | CF₃ | |
| Z1.142 | OH | CH₂OCH₂CH₂OCH₂CH₂OCH₃ | CF₃ | |
| Z1.143 | OH | CH₂OCH₂CH(OCH₃)₂ | CF₃ | |
| Z1.144 | OH | CH₂OCH₂CH(OCH₂CH₃)₂ | CF₃ | |
| Z1.145 | OH | 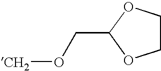 | CF₃ | |
| Z1.146 | OH | 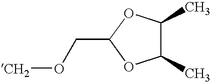 | CF₃ | |
| Z1.147 | OH | 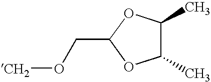 | CF₃ | |
| Z1.148 | OH | 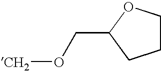 | CF₃ | |
| Z1.149 | OH | 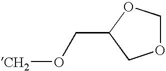 | CF₃ | |
| Z1.150 | OH | 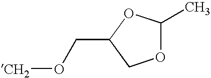 | CF₃ | |
| Z1.151 | OH | 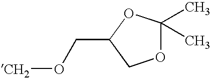 | CF₃ | |
| Z1.152 | OH | 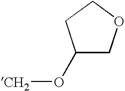 | CF₃ | |
| Z1.153 | OH | 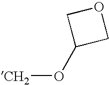 | CF₃ | |
| Z1.154 | OH | 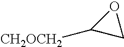 | CF₃ | |
| Z1.155 | OH | CH₂OCH₂CH₂SCH₃ | CF₃ | |
| Z1.156 | OH | CH₂OCH₂CH₂SCH₂CH₃ | CF₃ | |
| Z1.157 | OH | CH₂OCH₂CH₂S(O)₂CH₃ | CF₃ | |
| Z1.158 | OH | CH₂OCH₂CH₂S(O)₂CH₂CH₃ | CF₃ | |
| Z1.159 | OH | CH₂SCH₂CH₂OCH₃ | CF₃ | |
| Z1.160 | OH | CH₂SCH₂CH₂OCH₂CH₃ | CF₃ | |
| Z1.161 | OH | CH₂OCH₂CH₂OC(O)CH₃ | CF₃ | |
| Z1.162 | OH | CH₂OCH₂CH₂OC(O)-phenyl | CF₃ | |
| Z1.163 | OH | CH₂OCH₂CH₂OC(O)OCH₂CH₃ | CF₃ | |
| Z1.164 | OH | CH₂OCH₂CH₂OC(O)NHCH₂CH₃ | CF₃ | |
| Z1.165 | OH | CH₂OCH₂CH₂NH₂ | CF₃ | |
| Z1.166 | OH | CH₂OCH₂CH₂NHC(O)CH₃ | CF₃ | |
| Z1.167 | OH | CH₂OCH₂CH₂NHC(O)CH₂CH₃ | CF₃ | |
| Z1.168 | OH | CH₂OCH₂CH₂NHC(O)CH(CH₃)₂ | CF₃ | |
| Z1.169 | OH | CH₂OCH₂CH₂NHC(O)-cyclopropyl | CF₃ | |
| Z1.170 | OH | CH₂OCH₂CH₂NHC(O)C(CH₃)₃ | CF₃ | |
| Z1.171 | OH | CH₂OCH₂CH₂NHC(O)-phenyl | CF₃ | |
| Z1.172 | OH | CH₂OCH₂CH₂NHC(O)OCH₃ | CF₃ | |
| Z1.173 | OH | CH₂OCH₂CH₂NHC(O)OCH₂CH₃ | CF₃ | |
| Z1.174 | OH | CH₂OCH₂CH₂NHC(O)NHCH₃ | CF₃ | |
| Z1.175 | OH | CH₂OCH₂CH₂NHC(O)NHCH₂CH₃ | CF₃ | |
| Z1.176 | OH | CH₂OCH₂CH₂NHC(O)N(CH₃)₂ | CF₃ | |
| Z1.177 | OH | CH₂OCH₂CH₂NHC(O)N(CH₂CH₃)₂ | CF₃ | |
| Z1.178 | OH | CH₂N(SO₂CH₃)CH₃ | CF₃ | |
| Z1.179 | OH | CH₂N(SO₂CH₃)CH₂CH₃ | CF₃ | |
| Z1.180 | OH | CH₂N(SO₂CH₂CH₃)CH₂CH₃ | CF₃ | |
| Z1.181 | OH | CH₂N(SO₂CH₃)CH₂CF₃ | CF₃ | |
| Z1.182 | OH | CH₂N(SO₂CH₃)CH₂CHOCH₃ | CF₃ | |
| Z1.183 | OH | CH₂N(SO₂CH₃)CH₂-cyclopropyl | CF₃ | |
| Z1.184 | OH | CH₂N(SO₂CH₃)-phenyl | CF₃ | |
| Z1.185 | OH | CH₂N(SO₂CH₃)-benzyl | CF₃ | |
| Z1.186 | OH | CH₂N(SO₂CH₃)CH₂CH═CH₂ | CF₃ | |
| Z1.187 | OH | CH₂N(SO₂CH₃)CH₂C≡CH | CF₃ | |
| Z1.188 | OH | CH₂N(CH₃)C(O)H | CF₃ | |
| Z1.189 | OH | CH₂N(CH₃)C(O)CH₃ | CF₃ | |
| Z1.190 | OH | CH₂N(CH₃)C(O)CH₂CH₃ | CF₃ | |
| Z1.191 | OH | CH₂N(CH₃)C(O)-phenyl | CF₃ | |
| Z1.192 | OH | CH₂N(CH₃)C(O)-benzyl | CF₃ | |
| Z1.193 | OH | CH₂N(CH₂CH₃)C(O)CH₃ | CF₃ | |
| Z1.194 | OH | 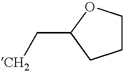 | CF₃ | |
| Z1.195 | OH | 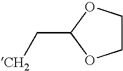 | CF₃ | |
| Z1.196 | OH | 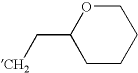 | CF₃ | |
| Z1.197 | OH | C(OCH₂CH₃)═CH₂ | CF₃ | amorphous solid |
| Z1.198 | OH | CH₂C(O)CH₃ | CF₃ | |
| Z1.199 | OH | C(OCH₃)₂ | CF₃ | |
| Z1.200 | OH | 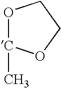 | CF₃ | amorphous solid |
| Z1.201 | OH | CH₂C(O)CH₂OCH₃ | CF₃ | |
| Z1.202 | OH | CH₂C(O)CH₂OCH₂CH₂OCH₃ | CF₃ | |
| Z1.203 | OH | CH₂C(O)CH₂N(SO₂CH₃)CH₃ | CF₃ | |
| Z1.204 | OH | C(CH₂OCH₃)═CH₂ | CF₃ | |
| Z1.205 | OH | 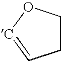 | CF₃ | |
| Z1.206 | OH | 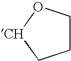 | CF₃ | |
| Z1.207 | OH | 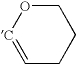 | CF₃ | amorphous solid |
| Z1.208 | OH | 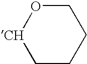 | CF₃ | |
| Z1.209 | OH | 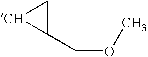 | CF₃ | |
| Z1.210 | OH | C(O)CH₃ | CF₃ | |
| Z1.211 | OH | C(O)CH₂OCH₃ | CF₃ | |
| Z1.212 | OH | C(O)CH₂OCH₂CH₂OCH₃ | CF₃ | |
| Z1.213 | OH | C(O)CH₂N(SO₂CH₃)CH₃ | CF₃ | |
| Z1.214 | OH | COOCH₃ | CF₃ | |
| Z1.215 | OH | COOCH₂CH₃ | CF₃ | |
| Z1.216 | OH | CH₃ | CF₃ | m.p.: 82-83° C. |
| Z1.217 | OH | benzylthio | OCHF₂ | m.p.: 132-133° C. |
| Z1.218 | OH | Cl | OCH₂CF₃ | m.p.: 131-133° C. |
| Z1.219 | OH | C≡N | CF₃ | m.p.: 122-123° C. |
| Z1.220 | OH | CH₃ | C≡N | m.p.: 157-158° C. |
| Z1.221 | OH | CF₃ | Cl | m.p.: 127-129° C. |
| Z1.222 | OEt | 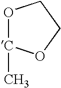 | CF₃ | waxy |
| Z1.223 | OEt | 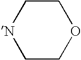 | CF₃ | oil, [P4] |
| Z1.224 | Cl | 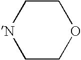 | CF₃ | m.p.: 72-73° C., [P6] |
| Z1.225 | OEt | N(CH₃)C(O)CH₃ | CF₃ | m.p.: 145-145.5° C., [P7] |
| Z1.226 | OEt | 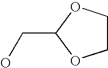 | CF₃ | oil, [P8] |
| Z1.227 | OEt | 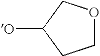 | CF₃ | m.p.: 45-45.5° C., [P9] |
| Z1.228 | OEt | 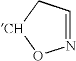 | CF₃ | oil |
| Z1.229 | OH | 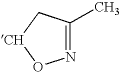 | CF₃ | amorphous solid |
| Z1.230 | OEt | 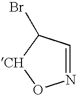 | CF₃ | oil |
| Z1.231 | OEt | 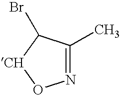 | CF₃ | oil |
| Z1.232 | OH | 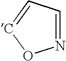 | CF₃ | solid |
| Z1.233 | OH | 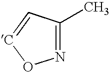 | CF₃ | amorphous solid |
| Z1.234 | OEt | 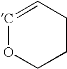 | CF₃ | oil |
| Z1.235 | OEt | 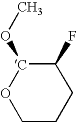 | CF₃ | oil, isomer I |
| Z1.236 | OEt | 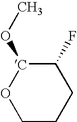 | CF₃ | oil, isomer II |
| Z1.237 | OEt | CH═CH₂ | CF₃ | oil, [P16] |
| Z1.238 | OEt | C(OCH₂CH₃)═CH₂ | CF₃ | oil, [P17] |
| Z1.239 | OEt | C(OCH₂CH₂CH₂CH₃)═CH₂ | CF₃ | |
| Z1.240 | OEt | CH═CHOCH₂CH₃ | CF₃ | |
| Z1.241 | OEt | CH═CHOCH₂CH₂CH₂CH₃ | CF₃ | |
| Z1.242 | OEt | C(O)CH₂Br | CF₃ | oil |
| Z1.243 | OEt | C(O)CH₂OH | CF₃ | oil |
| Z1.244 | OEt | CH₂CH₂OCH₂CH₃ | CF₃ | |
| Z1.245 | OH | CH₂CH₂OCH₂CH₂CH₂CH₃ | CF₃ | |
| Z1.246 | OH | CH₂O-phenyl | CF₃ | |
| Z1.247 | OH | CH₂NHSO₂-phenyl | CF₃ | |
| Z1.248 | OH | CH₂N(SO₂CH₃)-cyclopropyl | CF₃ | |
| Z1.249 | OH | CH₂N(SO₂CH₂CH₃)-phenyl | CF₃ | |
| Z1.250 | OH | CH₂NHC(O)-cyclopropyl | CF₃ | |
| Z1.251 | OH | CH₂NHC(O)-phenyl | CF₃ | |

### BIOLOGICAL EXAMPLES

### Example B1

### Herbicidal Action Prior to Emergence of the Plants (Pre-Emergence Action)

Monocotyledonous and dicotyledonous test plants are sown in standard soil in pots or seed trays. Immediately after sowing, the test compounds, in the form of an aqueous suspension (prepared from a wettable powder (Example F3, b) according to WO 97/34485) or in the form of an emulsion (prepared from an emulsifiable concentrate (Example F1, c) according to WO 97/34485), are applied by spraying in a concentration of 250 g/ha. The test plants are then grown in a greenhouse under optimum conditions. After a test duration of 4 weeks, the test is evaluated in accordance with a scale of eleven ratings (10=total damage, 0=no action). Ratings of from 10 to 7 (especially from 10 to 8) indicate very good to good herbicidal action.

TABLE B1
Pre-emergence action:

| Ex. No. | g/ha | Panicum | Digitaria | Echinochloa | Scirpus | Abutilon | Amaranthus | Chenopodium |
|---|---|---|---|---|---|---|---|---|
| 1.012 | 250 | 10 | 9 | 5 | 7 | 10 | 5 | 0 |
| 1.073 | 250 | 10 | 10 | 10 | nt | 10 | 6 | 10 |
| 1.075 | 250 | 10 | 10 | 10 | 7 | 9 | 10 | 9 |
| 1.079 | 250 | 10 | 10 | 10 | 4 | 10 | 10 | 9 |
| 1.126 | 250 | 9 | 6 | 2 | 8 | 10 | 10 | 10 |
| 2.021 | 250 | 9 | 3 | 0 | 7 | 9 | 7 | 9 |
| 2.059 | 250 | 7 | 7 | 8 | nt | 10 | 7 | 9 |
| 2.073 | 250 | 10 | 7 | 9 | 4 | 9 | 9 | 9 |
| 2.078 | 250 | 10 | 10 | 10 | 0 | 10 | 8 | 10 |
| 2.088 | 250 | 9 | 9 | 7 | 0 | 9 | nt | 7 |
| 2.089 | 250 | 9 | 8 | 8 | nt | 9 | 8 | 0 |
| 2.191 | 250 | 10 | 7 | 10 | 3 | 10 | 7 | 9 |
| 2.203 | 250 | 6 | 5 | 9 | 8 | 10 | 9 | nt |
| 2.209 | 250 | 10 | 8 | 10 | 2 | 10 | 9 | 9 |
| 3.069 | 250 | 10 | 10 | 10 | 5 | 10 | 10 | 6 |
| 3.071 | 250 | 10 | 10 | 10 | 8 | 9 | 8 | 6 |
| 3.072 | 250 | 9 | 10 | 10 | 7 | 9 | 3 | 8 |
| 3.073 | 250 | 10 | 9 | 7 | 7 | 5 | 0 | 7 |

### Example B2

### Post-Emergence Herbicidal Action

Monocotyledonous and dicotyledonous test plants are cultivated in a greenhouse in standard soil in plastic pots and, at the 4- to 6-leaf stage, are sprayed with an aqueous suspension of the test compounds of formula I prepared from a 25% wettable powder (Example F3, b) according to WO 97/34485) or with an emulsion of the test compounds of formula I prepared from a 25% emulsifiable concentrate (Example F1, c) according to WO 97/34485), corresponding to concentrations of 125 and 250 g of active ingredient per hectare (500 litres of water per ha). The test plants are then grown on in the greenhouse under optimum conditions. After a test duration of about 18 days, the test is evaluated in accordance with a scale of eleven ratings (10=total damage, 0=no action). Ratings of from 10 to 7 (especially from 10 to 8) indicate very good to good herbicidal action. The compounds of formula I generally exhibit a strong herbicidal action in this test.

TABLE B2
Post-emergence action

| Ex. No. | g/ha | Panicum | Echinochloa | Euphorbia | Xanthium | Amaranthus | Chenopodium | Stellaria |
|---|---|---|---|---|---|---|---|---|
| 1.003 | 250 | 8 | 8 | 6 | 9 | 9 | 10 | 10 |
| 1.012 | 250 | 7 | 8 | 9 | 8 | 9 | 8 | 7 |
| 1.021 | 250 | 6 | 6 | 7 | 9 | 9 | 8 | 7 |
| 1.073 | 250 | 10 | 9 | 9 | 9 | 10 | 8 | 9 |
| 1.075 | 250 | 10 | 8 | 8 | 7 | 10 | 10 | 8 |
| 1.079 | 250 | 3 | 7 | 8 | 7 | 8 | 10 | 10 |
| 1.081 | 250 | 10 | 9 | 9 | 9 | 10 | 9 | 6 |
| 1.090 | 250 | 8 | 7 | nt | 8 | 8 | 9 | 6 |
| 1.126 | 250 | 7 | 7 | 8 | 8 | 8 | 8 | 8 |
| 1.203 | 250 | 9 | 8 | 7 | 8 | 8 | 9 | 7 |
| 2.003 | 250 | 9 | 9 | 9 | 8 | 8 | 8 | 9 |
| 2.021 | 250 | 9 | 9 | 9 | 8 | 9 | 8 | 7 |
| 2.059 | 250 | 5 | 8 | 6 | 7 | 7 | 10 | 8 |
| 2.073 | 250 | 9 | 9 | 9 | 9 | 9 | 8 | 7 |
| 2.078 | 250 | 5 | 8 | 7 | 7 | 7 | 10 | 10 |
| 2.080 | 250 | 9 | 9 | 9 | 9 | 9 | 6 | 7 |
| 2.089 | 250 | 8 | 7 | 8 | 7 | 0 | 9 | 8 |
| 2.094 | 250 | 8 | 8 | 8 | 7 | 3 | 9 | 9 |
| 2.095 | 250 | 9 | 9 | 9 | 9 | 9 | 8 | 5 |
| 2.188 | 250 | 9 | 7 | 7 | 8 | 5 | 6 | 10 |
| 2.191 | 250 | 9 | 8 | 8 | 8 | 3 | 7 | 10 |
| 2.203 | 250 | 9 | 9 | 9 | 9 | 6 | 6 | 9 |
| 2.208 | 250 | 9 | 9 | 8 | 8 | 8 | 6 | 5 |
| 2.209 | 250 | 9 | 8 | 8 | 9 | 8 | 6 | 10 |
| 2.201 | 250 | 9 | 8 | 6 | 8 | 9 | 8 | 6 |
| 3.068 | 250 | 10 | 9 | 9 | 9 | 10 | 9 | 9 |
| 3.069 | 250 | 8 | 7 | 5 | 8 | 8 | 10 | 7 |
| 3.070 | 250 | 7 | 8 | 7 | 5 | 7 | 9 | 5 |
| 3.071 | 250 | 9 | 8 | 7 | 8 | 8 | 10 | 8 |
| 3.072 | 250 | 8 | 7 | 7 | 8 | 7 | 9 | 8 |
| 3.073 | 250 | 7 | 8 | 7 | 7 | 4 | 9 | 7 |
| 3.074 | 250 | 7 | 7 | 7 | 8 | 2 | 9 | 8 |
| 3.075 | 250 | 5 | 7 | 7 | 8 | 2 | 9 | 8 |
| 3.076 | 250 | 7 | 7 | 6 | 8 | 7 | 9 | 7 |
| 3.212 | 250 | 0 | 5 | 7 | 8 | 8 | 7 | 7 |

### Example B3

### Microscreen, Post-Emergence Herbicidal Action

Monocotyledonous and dicotyledonous test plants are sown in sterilised standard soil in seed trays each having 96 cells. After 8 to 9 days' cultivation under controlled conditions in a climatic chamber (cultivation at 17/23° C.; 13 hours' light; 50-60% humidity; after application at 19/24° C.), the plants are treated with an aqueous spray solution of 1000 mg/l of the active ingredient used (rate of application: 500 g/l; incl. 10% DMSO as solvent). The plants are grown on in the climatic chamber until the test is evaluated after 9 or 13 days. The test is evaluated in accordance with a scale of eleven ratings (10=total damage, 0=no action). Ratings of from 10 to 7 (especially from 10 to 8) indicate very good to good herbicidal action. The compounds of formula I generally exhibit a strong herbicidal action in this test.

TABLE B3

| Ex. | | | Ama- | | | |
|---|---|---|---|---|---|---|
| No. | g/ha | Digitaria | ranthus | Solanum | Nasturtium | Stellaria |
| 1.059 | 250 | 7 | 7 | 9 | 9 | 9 |
| 1.060 | 250 | 4 | 8 | 8 | 9 | 7 |
| 1.080 | 250 | 5 | 7 | 9 | 10 | 9 |
| 1.082 | 250 | 3 | 6 | 9 | 10 | 10 |
| 1.091 | 250 | 2 | 8 | 9 | 9 | 6 |
| 1.094 | 250 | 8 | 9 | 10 | 8 | 7 |
| 1.095 | 250 | 3 | 7 | 7 | 9 | 7 |
| 1.096 | 250 | 8 | 9 | 9 | 10 | 9 |
| 1.189 | 250 | 7 | nt | 9 | 9 | 8 |
| 1.192 | 250 | 7 | 8 | 9 | 10 | 9 |
| 1.204 | 250 | 8 | 10 | 10 | 10 | 10 |
| 1.209 | 250 | 8 | 9 | 9 | 9 | 9 |
| 1.211 | 250 | 5 | 8 | 8 | 10 | 8 |
| 1.212 | 250 | 3 | 7 | 8 | 8 | 7 |
| 2.010 | 250 | 3 | 8 | 9 | 9 | 8 |
| 2.060 | 250 | 9 | 9 | 10 | 10 | 10 |
| 2.064 | 250 | 9 | 8 | 10 | 10 | 10 |
| 2.075 | 250 | 7 | 6 | 10 | 10 | 9 |
| 2.090 | 250 | 2 | 6 | 10 | 10 | 8 |
| 2.202 | 250 | 6 | 6 | 10 | 10 | 10 |

### Example B4

### Comparison Test with a Compound from the Prior Art: Post-Emergence Herbicidal Action

The post-emergence herbicidal action of compound no. 1.095 according to the invention was compared with compound A, which is described as compound no. 1.005 on page 15, Table 1, of EP-A-0 353 187:

TABLE B4

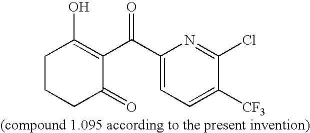

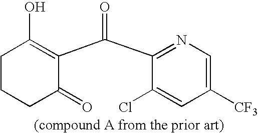

| Comp. | | | | | | | | | |
|---|---|---|---|---|---|---|---|---|---|
| No. | g/ha | Wheat | Maize | Sida | Ipomea | Amaranthus | Sinapis | Stellaria | Galium |
| 1.095 | 125 | 0 | 0 | 5 | 7 | 7 | 7 | 8 | 6 |
| A | 125 | 0 | 0 | 2 | 2 | 2 | 6 | 9 | 1 |

The results in Table B4 show that, at an application rate of 125 g/ha, compound no. 1.095 according to the invention exerts a generally substantially better herbicidal action on the tested weeds than compound A from the prior art. The improved action can be seen especially clearly in the case of the weeds Ipomea, Amaranthus and Galium, where the action of compound A from the prior art is completely inadequate at the tested rates of application. Only in the case of Stellaria does compound A from the prior art exhibit slightly better action. In view of the structural similarity between the compounds, the enhanced action of the compounds according to the invention was not to be expected.

### Example B5

### Comparison Test with a Compound from the Prior Art: Post-Emergence Herbicidal Action

The post-emergence herbicidal action of compound no. 1.096 according to the invention was compared with compound B, which is described as compound no. 2.088 on page 83,

TABLE B5

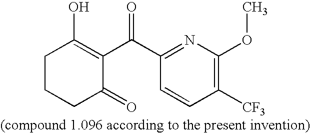

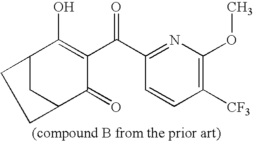

| Comp. | | | | | | | | | |
|---|---|---|---|---|---|---|---|---|---|
| No. | g/ha | Wheat | Maize | Bromus | Sida | Ipomea | Polygonum | Stellaria | Galium |
| 1.096 | 125 | 0 | 0 | 5 | 7 | 7 | 8 | 8 | 8 |
| B | 125 | 2 | 2 | 0 | 0 | 6 | 4 | 6 | 4 |

The results in Table B4 show that, at an application rate of 125 g/ha, compound no. 1.096 according to the invention exerts a substantially better herbicidal action on the tested weeds than compound B from the prior art. The improved action can be seen especially clearly in the case of the weeds Bromus and Sida, where compound B from the prior art has no action at all at the tested rates of application. In addition, the compound according to the invention not only exhibits better herbicidal action but also exhibits no phytotoxicity whatsoever with respect to the useful plants (in this case, wheat and maize). At an application rate of 125 g/ha, compound B from the prior art already shows significant damage (20% phytotoxicity) to wheat and maize, which is unacceptable from an agronomic standpoint. In view of the structural similarity between the compounds, the enhanced action and improvement in selectivity of the compounds according to the invention were not to be expected.

What is claimed is:

1. A compound of formula I 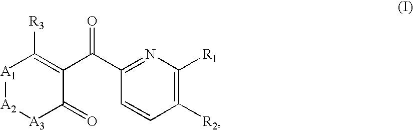
wherein
R₁ is -L₁₀-R₄, -L₁₁-X₁—R₅, —NR₆R₇, —X₂—R₈ or —X₃-L₁-R₉;
L₂, L₄, L₆ and L₈ are each independently of the others C₁-C₄alkylene which may be substituted once, twice or three times by C₁-C₄alkyl, halogen or by C₁-C₄alkoxy and to which C₁-C₄alkylene group there may additionally be spirocyclically bound a C₂-C₅alkylene group, and wherein that C₂-C₅alkylene group may in turn be interrupted once or twice by oxygen, sulfur, sulfinyl or by sulfonyl and/or substituted by C₁-C₄alkyl or by C₁-C₄alkoxy;
L₃, L₅, L₇ and L₉ are each independently of the others C₁-C₄alkylene which may be substituted once, twice or three times by C₁-C₄alkyl, halogen or by C₁-C₄alkoxy;
R₂ is halogen, C₁-C₄haloalkyl, cyano, C₁-C₃haloalkoxy, C₁-C₄alkylthio, C₁-C₄alkylsulfinyl, C₁-C₄alkylsulfonyl, C₁-C₄haloalkylthio, C₁-C₄haloalkylsulfinyl or C₁-C₄haloalkylsulfonyl;
L₁₀ is a direct bond or a C₁-C₆alkylene, C₂-C₆alkenylene or C₂-C₆alkynylene group which may be substituted once, twice or three times by C₁-C₆alkyl, halogen, hydroxy, C₁-C₆alkoxy, C₃-C₆cycloalkyloxy, C₁-C₆alkoxy-C₁-C₆alkoxy, C₁-C₆alkoxy-C₁-C₆alkoxy-C₁-C₆alkoxy or by C₁-C₂alkylsulfonyloxy;
R₄ is halogen, cyano, rhodano, C₁-C₆alkoxycarbonyl, C₃-C₆alkenyloxycarbonyl, C₃-C₆alkynyl-oxycarbonyl, benzyloxycarbonyl, C(O)NR₂₅ₐR₂₆ₐ, formyl, C₁-C₆alkylcarbonyl, C₁-C₆haloalkylcarbonyl, C₁-C₄alkoxy-C₁-C₄alkylcarbonyl, C₁-C₄alkoxy-C₁-C₄alkoxy-C₁-C₄alkylcarbonyl, N—(C₁-C₄alkyl)-C₁-C₄alkylsulfonylamino-C₁-C₄alkylcarbonyl, C₁-C₆haloalkyl, C₂-C₆alkenyl, C₂-C₆-haloalkenyl, C₂-C₆alkynyl, C₂-C₆haloalkynyl, C₃-C₆cycloalkyl, C₁-C₆alkylsulfonyloxy or phenylsulfonyloxy, wherein the phenyl groups may be substituted by one or more C₁-C₆alkyl, C₁-C₆-haloalkyl, C₁-C₆alkoxy, C₁-C₆haloalkoxy, halogen, cyano, hydroxy or nitro groups;
or R₄ is a three- to ten-membered, monocyclic or fused bicyclic ring system which may be aromatic, saturated or partially saturated and which may contain from 1 to 4 hetero atoms selected from nitrogen, oxygen and sulfur, and wherein the ring system may contain not more than 2 oxygen atoms and not more than two sulfur atoms, and each ring system may itself be substituted once, twice or three times by C₁-C₆alkyl, C₁-C₆haloalkyl, C₁-C₄alkoxy-C₁-C₂alkyl, C₂-C₆alkenyl, C₂-C₆haloalkenyl, C₂-C₆alkynyl, C₂-C₆haloalkynyl, C₁-C₆alkoxy, hydroxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di(C₁-C₂alkyl)aminosulfonyl, di(C₁-C₄alkyl)amino, halogen, cyano, nitro, phenyl or by benzylthio, and wherein phenyl and benzylthio may in turn be substituted on the phenyl ring by C₁-C₃alkyl, C₁-C₃haloalkyl, C₁-C₃alkoxy, C₁-C₃haloalkoxy, halogen, cyano or by nitro, and wherein the substituent on the nitrogen in the heterocyclic ring are other than halogen;
or R₄ is hydrogen when L₁₀ is a C₁-C₆alkylene group which may be substituted once, twice or three times by C₁-C₆alkyl or by halogen; or when L₁₀ is a C₂-C₆alkenylene or C₂-C₆alkynylene group which may be substituted once, twice or three times by C₁-C₆alkyl, halogen, hydroxy, C₁-C₆alkoxy, C₃-C₆cycloalkyloxy, C₁-C₆alkoxy-C₁-C₆alkoxy, C₁-C₆alkoxy-C₁-C₆alkoxy-C₁-C₆-alkoxy or by C₁-C₂alkylsulfonyloxy;
R₂₅ₐ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₂₆ₐ is hydrogen, C₁-C₆alkyl, C₃-C₆alkenyl or C₃-C₆alkynyl;
or R₂₅ₐ together with R₂₆ₐ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
L₁₁ is a C₁-C₆alkylene, C₂-C₆alkenylene or C₂-C₆alkynylene group which may be substituted once, twice or three times by halogen, hydroxy, C₁-C₆alkoxy, C₃-C₆cycloalkyloxy, C₁-C₆-alkoxy-C₁-C₆alkoxy, C₁-C₆alkoxy-C₁-C₆alkoxy-C₁-C₆alkoxy or by C₁-C₂alkylsulfonyloxy;
X₁ is oxygen, —OC(O)—, —C(O)—, —C(═NR₁₄ₐ)—, —C(O)O—, —C(O)NR_{14b}—, —OC(O)O—, —N(R₁₀)—O—, —O—NR₁₁—, thio, sulfinyl, sulfonyl, —SO₂NR₁₂—, —NR₁₃SO₂—, —N(SO₂R_{14c})—, —N(R_{14d})C(O)— or —NR₁₄—;
R₁₀, R₁₁, R₁₂, R₁₃, R_{14b}, R_{14d} and R₁₄ are each independently of the others hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl, or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;
R₁₄ₐ is hydroxy, C₁-C₆alkoxy, C₃-C₆alkenyloxy, C₃-C₆alkynyloxy or benzyloxy;
R_{14c} is C₁-C₆alkyl;
R₅ is hydrogen or a C₁-C₈alkyl, C₃-C₈alkenyl or C₃-C₆alkynyl or C₃-C₆cycloalkyl group which may be substituted once, twice or three times by chlorine, bromine, iodine, hydroxy, amino, formyl, nitro, cyano, mercapto, C₁-C₆alkoxy, C₂-C₆alkenyl, C₂-C₆haloalkenyl, C₂-C₆alkynyl, C₂-C₆haloalkynyl, C₃-C₆cycloalkyl, halo-substituted C₃-C₆cycloalkyl, C₃-C₆alkenyloxy, C₃-C₆alkynyloxy, C₁-C₆haloalkoxy, C₃-C₆haloalkenyloxy, cyano-C₁-C₆alkoxy, C₁-C₆alkoxy-C₁-C₆alkoxy, C₁-C₆alkoxy-C₁-C₆alkoxy-C₁-C₆alkoxy, C₁-C₆alkylthio-C₁-C₆alkoxy, C₁-C₆alkylsulfinyl-C₁-C₆alkoxy, C₁-C₆alkylsulfonyl-C₁-C₆alkoxy, C₁-C₆alkoxycarbonyl-C₁-C₆alkoxy, C₁-C₆alkoxycarbonyl, C₁-C₆alkylcarbonyl, phenylcarbonyl, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzyloxy, benzylthio, benzylsulfinyl, benzylsulfonyl, C₁-C₆alkylamino, di(C₁-C₆alkyl)amino, R₁₅ₐC(X₂₃)N(R₁₈ₐ)—, R₁₆ₐN(R₁₇ₐ)C(X₂₄)—, R_{16b}N(R_{17b})C(X₂₅)NR_{18b}—, R_{15c}SO₂N(R_{18c})—, R_{16c}N(R_{17c})C(X₂₆)O—, R_{15b}C(X₂₇)O—, R₁₉R₂₀C═NO—, R₁₅S(O)₂O—, R₁₆N(R₁₇)SO₂—, rhodano, phenyl, phenoxy, phenylthio, phenylsulfinyl or by phenylsulfonyl or which may be substituted from one to seventeen times by fluorine; wherein the phenyl- or benzyl-containing groups may in turn be substituted by one or more C₁-C₆alkyl, C₁-C₆haloalkyl, C₁-C₆alkoxy, C₁-C₆haloalkoxy, halogen, cyano, hydroxy or nitro groups;
R₁₅ₐ, R_{15b} and R_{15c} are hydrogen, C₁-C₈alkyl, C₂-C₆alkenyl, C₃-C₆cycloalkyl, phenyl, benzyl, C₁-C₆alkoxy, C₃-C₆alkenyloxy, C₃-C₆alkynyloxy or benzyloxy, wherein the phenyl groups may be substituted once, twice or three times by C₁-C₆alkyl, C₁-C₆haloalkyl, C₁-C₆alkoxy, C₁-C₆haloalkoxy, halogen, cyano, hydroxy or by nitro;
R₁₆ₐ, R_{16b} and R_{16c} are hydrogen, C₁-C₆alkyl, C₃-C₆alkenyl, C₃-C₆alkynyl, C₃-C₆cycloalkyl or phenyl, wherein phenyl may be substituted once, twice or three times by C₁-C₆alkyl, C₁-C₆haloalkyl, C₁-C₆alkoxy, C₁-C₆haloalkoxy, halogen, cyano, hydroxy or by nitro;
R₁₇ₐ, R_{17b}, R_{17c}, R₁₈ₐ, R_{18b} and R_{18c} are hydrogen, C₁-C₆alkyl, C₃-C₆alkenyl or C₃-C₆alkynyl;
X₂₃, X₂₄, X₂₅, X₂₆ and X₂₇ are oxygen or sulfur;
R₁₅, R₁₆, R₁₇, R₁₉ and R₂₀ are each independently of the others hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl, or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;
or R₅ is a three- to ten-membered monocyclic or fused bicyclic ring system which may be aromatic, saturated or partially saturated and may contain from 1 to 4 hetero atoms selected from nitrogen, oxygen and sulfur, and wherein the ring system is bound to the substituent X₁ directly or via a C₁-C₄alkylene, C₂-C₄alkenylene, C₂-C₄alkynylene, —N(R₁₈)—C₁-C₄alkylene, —O—C₁-C₄alkylene, —S—C₁-C₄alkylene, —S(O)—C₁-C₄alkylene or —SO₂—C₁-C₄alkylene chain, wherein the ring system may not be interrupted by —C(═O)—, —C(═S)—, —C(═NR₅ₐ)—, —N(═O)—, —S(═O)— or by —SO₂—, and each ring system may contain not more than 2 oxygen atoms and not more than two sulfur atoms, and the ring system itself may be substituted once, twice or three times by C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, hydroxy, C₁-C₆haloalkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆-alkynylthio, C₂-C₅alkoxyalkylthio, C₃-C₅acetylalkylthio, C₃-C₆alkoxycarbonylalkylthio, C₂-C₄-cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di(C₁-C₂alkyl)aminosulfonyl, di(C₁-C₄alkyl)amino, halogen, cyano, nitro, phenyl or by benzylthio, wherein phenyl and benzylthio may in turn be substituted on the phenyl ring by C₁-C₃alkyl, C₁-C₃haloalkyl, C₁-C₃alkoxy, C₁-C₃-haloalkoxy, halogen, cyano or by nitro, and wherein the substituents on the nitrogen in the heterocyclic ring are other than halogen;
R₅ₐ is C₁-C₆alkyl, hydroxy, C₁-C₆alkoxy, cyano or nitro;
R₁₈ is hydrogen, C₁-C₆alkyl, C₁-C₆haloalkyl, C₁-C₆alkoxycarbonyl, C₁-C₆alkylcarbonyl, C₁-C₆alkoxy-C₁-C₆alkyl, or C₁-C₆alkoxy-C₁-C₆alkyl substituted by C₁-C₆alkoxy, or benzyl or phenyl, wherein phenyl and benzyl may in turn be substituted once, twice or three times by C₁-C₆alkyl, C₁-C₆haloalkyl, C₁-C₆alkoxy, C₁-C₆haloalkoxy, halogen, cyano, hydroxy or by nitro;
R₆ is hydrogen, C₁-C₆alkyl, C₃-C₆alkenyl, C₃-C₆alkynyl, C₁-C₆haloalkyl, hydroxy, C₁-C₆alkoxy, —C(O)R₁₉ₐ or —C(S)R₂₀ₐ;
R₁₉ₐ and R₂₀ₐ are each independently of the other hydrogen, C₁-C₆alkyl, C₃-C₆cycloalkyl, phenyl, benzyl, heteroaryl, C₁-C₆alkoxy, C₃-C₆alkenyloxy, benzyloxy, C₁-C₄alkylthio or a group NR₂₁R₂₂;
R₂₁ and R₂₂ are each independently of the other hydrogen, C₁-C₆alkyl, C₃-C₆alkenyl, C₃-C₆alkynyl or phenyl, and wherein phenyl, benzyl, benzyloxy and heteroaryl in R₁₉ₐ, R₂₀ₐ, R₂₁ and R₂₂ may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₂₁ together with R₂₂ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₆ is -L₂-X₄—R₂₄; wherein
X₄ is oxygen, —NR₂₃—, —S—, —S(O)— or —S(O)₂—;
R₂₃ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₂₄ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₅)NR₂₅R₂₆, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₂₄ is C(O)—R₇₄ or C(S)—R₇₅;
X₅ is oxygen or sulfur;
R₂₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₂₆ is hydrogen, C₁-C₆alkyl, C₃-C₆alkenyl or C₃-C₆alkynyl;
or R₂₅ together with R₂₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₆ is -L₃-R₂₇;
R₂₇ is formyl, C₁-C₆alkylcarbonyl, C₃-C₆cycloalkylcarbonyl, benzoyl, C₁-C₆alkoxycarbonyl, cyano, C(X₆)NR₂₈R₂₉, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;
or R₂₇ is C₃-C₆cycloalkyl or C₅-C₆cycloalkenyl each of which may in turn be substituted once, twice or three times by C₁-C₄alkyl, halogen or by C₁-C₄alkoxy;
X₆ is oxygen or sulfur;
R₂₈ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₂₉ is hydrogen, C₁-C₆alkyl, C₃-C₆alkenyl or C₃-C₆alkynyl;
or R₂₈ together with R₂₉ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₇ is hydrogen, C₁-C₆alkyl, C₃-C₆alkenyl, C₃-C₆alkynyl, C₁-C₆haloalkyl, C₃-C₆cycloalkyl, phenyl, benzyl, heteroaryl, C(X₇)R₃₀ or NR₃₃R₃₄, wherein phenyl, benzyl and heteroaryl may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
X₇ is oxygen or sulfur;
R₃₀ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₃₁R₃₂;
R₃₁ and R₃₃ are each independently of the other hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₃₂ and R₃₄ are each independently of the other hydrogen, C₁-C₆alkyl, C₃-C₆alkenyl or C₃-C₆alkynyl;
or R₃₁ together with R₃₂ or R₃₃ together with R₃₄, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₇ is -L₄-X—R₃₅; wherein
X₈ is oxygen, —NR₃₆—, —S—, —S(O)— or —S(O)₂—;
R₃₆ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₃₅ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₉)NR₃₇R₃₈, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
X₉ is oxygen or sulfur;
R₃₇ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₃₈ is hydrogen, C₁-C₆alkyl, C₃-C₆alkenyl or C₃-C₆alkynyl;
or R₃₇ together with R₃₈ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₇ is -L₅-R₃₉;
R₃₉ is formyl, C₁-C₆alkylcarbonyl, C₃-C₆cycloalkylcarbonyl, benzoyl, C₁-C₆alkoxycarbonyl, cyano, C(X₁₀)NR₄₀R₄₁, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;
or R₃₉ is C₃-C₆cycloalkyl or C₅-C₆cycloalkenyl each of which may in turn be substituted once, twice or three times by C₁-C₄alkyl, halogen or by C₁-C₄alkoxy;
X₁₀ is oxygen or sulfur;
R₄₀ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₄₁ is hydrogen, C₁-C₆alkyl, C₃-C₆alkenyl or C₃-C₆alkynyl;
or R₄₀ together with R₄₁ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₆ and R₇ together with the nitrogen atom to which they are bonded form a carbocyclic 3- to 7-membered, saturated or partially saturated or unsaturated monocyclic or bicyclic ring system which may be interrupted once by oxygen, once by sulfur, from one to three times by nitrogen and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃-haloalkylthio, cyano, nitro or by C₁-C₄alkoxycarbonyl; wherein each ring system may not be interrupted by —C(═O)—, —C(═S)—, —C(═NR₅ₐ)—, —N(═O)—, —S(═O)— or by —SO₂—;
R₅ₐ is C₁-C₆alkyl, hydroxy, C₁-C₆alkoxy, cyano or nitro;
X₂ is oxygen, —NR₄₂—, sulfur, —S(O)— or —S(O)₂—;
R₄₂ is hydrogen, C₁-C₆alkyl, C₃-C₆alkenyl, C₃-C₆alkynyl, C₁-C₆haloalkyl, C₃-C₆cycloalkyl, phenyl, heteroaryl, C(X₁₁)R₄₃ or NR₄₆R₄₇;
X₁₁ is oxygen or sulfur;
R₄₃ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₄₄R₄₅;
R₄₄ and R₄₆ are each independently of the other hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₄₅ and R₄₇ are each independently of the other hydrogen, C₁-C₆alkyl, C₃-C₆alkenyl or C₃-C₆alkynyl;
or R₄₄ together with R₄₅ or R₄₆ together with R₄₇, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₄₂ is -L₆-X₁₂—R₄₈; wherein
X₁₂ is oxygen, —NR₄₉—, —S—, —S(O)— or —S(O)₂—;
R₄₉ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₄₈ is a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆alkylsulfonyl, cyano, C(X₁₃)NR₅₀R₅₁, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
X₁₃ is oxygen or sulfur;
R₅₀ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₅₁ is hydrogen, C₁-C₆alkyl, C₃-C₆alkenyl or C₃-C₆alkynyl;
or R₅₀ together with R₅₁ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₄₂ is -L₇-R₅₂;
R₅₂ is formyl, C₁-C₆alkylcarbonyl, C₃-C₆cycloalkylcarbonyl, benzoyl, C₁-C₆alkoxycarbonyl, cyano, C(X₁₄)NR₅₃R₅₄, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;
or R₅₂ is C₃-C₆cycloalkyl or C₅-C₆cycloalkenyl each of which may in turn be substituted once, twice or three times by C₁-C₄alkyl, halogen or by C₁-C₄alkoxy;
X₁₄ is oxygen or sulfur;
R₅₃ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₅₄ is hydrogen, C₁-C₆alkyl, C₃-C₆alkenyl or C₃-C₆alkynyl;
or R₅₃ together with R₅₄ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃-haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₈ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₁₅)NR₅₅R₅₆, C₃-C₆cycloalkyl, phenyl, phenoxy or by 5- or 6-membered heteroaryl or heteroaryloxy, and wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
X₁₅ is oxygen or sulfur;
R₅₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₅₆ is hydrogen, C₁-C₆alkyl, C₃-C₆alkenyl or C₃-C₆alkynyl;
or R₅₅ together with R₅₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₈ is cyano, C(O)—R₇₆ or C(S)—R₇₇;
X₃ is oxygen, —NR₅₇—, sulfur, —S(O)— or —S(O)₂—;
R₅₇ is hydrogen, C₁-C₆alkyl, C₃-C₆alkenyl, C₃-C₆alkynyl, C₁-C₆haloalkyl, C₃-C₆cycloalkyl, phenyl, heteroaryl, C(X₁₆)R₅₈ or NR₆₁R₆₂;
X₁₆ is oxygen or sulfur;
R₅₈ is hydrogen, C₁-C₆alkyl, C₃-C₆cycloalkyl, phenyl, heteroaryl, C₁-C₆alkoxy, C₃-C₆alkenyloxy, benzyloxy, C₁-C₄alkylthio or a group NR₅₉R₆₀;
R₅₉ and R₆₁ are each independently of the other hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₆₀ and R₆₂ are each independently of the other hydrogen, C₁-C₆alkyl, C₃-C₆alkenyl or C₃-C₆alkynyl;
or R₅₉ together with R₆₀ or R₆₁ together with R₆₂, in each case with the respective N atom to which they are bonded, form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₅₇ is -L₈-X₁₇—R₆₃; wherein
X₁₇ is oxygen, —NR₆₄—, —S—, —S(O)— or —S(O)₂—;
R₆₄ is hydrogen, C₁-C₆alkoxy, C₁-C₆alkyl, C₃-C₆alkenyl or C₃-C₆alkynyl or is phenyl which may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₆₃ is a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆alkylsulfonyl, cyano, C(X₁₈)NR₆₅R₆₆, C₃-C₆cycloalkyl, phenyl, phenoxy or by 5 or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
X₁₈ is oxygen or sulfur;
R₆₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₆₆ is hydrogen, C₁-C₆alkyl, C₃-C₆alkenyl or C₃-C₆alkynyl;
or R₆₅ together with R₆₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₅₇ is -L₉-R₆₇;
R₆₇ is formyl, C₁-C₆alkylcarbonyl, C₃-C₆cycloalkylcarbonyl, benzoyl, C₁-C₆alkoxycarbonyl, cyano, C(X₁₉)NR₆₈R₆₉, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl; or R₆₇ is C₃-C₆cycloalkyl or C₅-C₆cycloalkenyl each of which may in turn be substituted once, twice or three times by C₁-C₄alkyl, halogen or by C₁-C₄alkoxy;
X₁₉ is oxygen or sulfur;
R₆₈ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₆₉ is hydrogen, C₁-C₆alkyl, C₃-C₆alkenyl or C₃-C₆alkynyl;
or R₆₈ together with R₆₉ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
L₁ is C₁-C₄alkylene which may be substituted once, twice or three times by C₁-C₄alkyl, halogen or by C₁-C₄alkoxy and to which C₁-C₄alkylene group there may be spirocyclically bound a further C₂-C₅alkylene group which may in turn be interrupted once or twice by oxygen, sulfur, sulfinyl or by sulfonyl and/or substituted by C₁-C₄alkyl or by C₁-C₄alkoxy;
or L₁ is C₁-C₄alkylene which may be substituted once, twice or three times by C₁-C₄alkyl, halogen or by C₁-C₄alkoxy, and wherein a carbon atom of that C₁-C₄alkylene group together with R₉ or with R₇₀ forms, by means of a further C₂-C₆alkylene chain, a ring system which may additionally be interrupted once or twice by oxygen, sulfur, sulfinyl or by sulfonyl and/or substituted by C₁-C₄alkyl or by C₁-C₄alkoxy; R₉ is a group —X₂₀—R₇₀, wherein
X₂₀ is oxygen, —NR₇₁—, —S—, —S(O)— or —S(O)₂—;
R₇₁ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₂₁)NR₇₂R₇₃, C₃-C₆cycloalkyl, phenyl, phenoxy or by 5- or 6-membered heteroaryl or heteroaryloxy, wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
X₂₁ is oxygen or sulfur;
R₇₂ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₇₃ is hydrogen, C₁-C₆alkyl, C₃-C₆alkenyl or C₃-C₆alkynyl;
or R₇₂ together with R₇₃ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₇₀ is hydrogen or a C₁-C₆alkyl, C₃-C₆alkenyl or C₃-C₆alkynyl group, which groups may be substituted once, twice or three times by halogen, hydroxy, C₁-C₆alkoxy, C₁-C₃alkoxy-C₁-C₃alkoxy, C₃-C₆alkenyloxy, C₃-C₆alkynyloxy, C₁-C₆alkylthio, C₁-C₆alkylsulfinyl, C₁-C₆-alkylsulfonyl, cyano, C(X₁₅ₐ)NR₅₅ₐR₅₆ₐ, C₃-C₆cycloalkyl, phenyl, phenoxy or by 5- or 6-membered heteroaryl or heteroaryloxy, and wherein heteroaryl or heteroaryloxy may in turn be interrupted once by oxygen or by sulfur or once, twice or three times by nitrogen and may be bonded to the C₁-C₆alkyl, C₃-C₆alkenyl or C₃-C₆alkynyl group either via a C atom or via a N atom, and wherein the phenyl- and heteroaryl-containing groups may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
X₁₅ₐ is oxygen or sulfur;
R₅₅ₐ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₅₆ₐ is hydrogen, C₁-C₆alkyl, C₃-C₆alkenyl or C₃-C₆alkynyl;
or R₅₅ₐ together with R₅₆ₐ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₇₀ is C₁-C₁₀alkylideneimino, (phenyl-C₁-C₄alkylidene)imino, or phenyl, wherein phenyl may be substituted once, twice or three times by halogen, C₁-C₄alkyl, C₁-C₄haloalkyl, C₁-C₃-alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
or R₇₀ is cyano, C(O)—R₇₈ or C(S)—R₇₉;
or R₉ is formyl, C₁-C₆alkylcarbonyl, C₃-C₆cycloalkylcarbonyl, benzoyl, C₁-C₆alkoxycarbonyl, cyano, C(X₃₅)NR₁₂₅R₁₂₆, phenyl or heteroaryl, wherein benzoyl and phenyl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
and wherein heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro or by C₁-C₄alkoxycarbonyl;
or R₉ is C₃-C₆cycloalkyl or C₅-C₆cycloalkenyl each of which may in turn be substituted once, twice or three times by C₁-C₄alkyl, halogen or by C₁-C₄alkoxy;
X₃₅ is oxygen or sulfur;
R₁₂₅ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₁₂₆ is hydrogen, C₁-C₆alkyl, C₃-C₆alkenyl or C₃-C₆alkynyl;
or R₁₂₅ together with R₁₂₆ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₇₄, R₇₅, R₇₆, R₇₇, R₇₈ and R₇₉ are each independently of the others hydrogen, C₁-C₆alkyl, C₃-C₆cycloalkyl, phenyl, benzyl, heteroaryl, C₁-C₆alkoxy, C₃-C₆alkenyloxy, benzyloxy, C₁-C₄alkylthio or NR₁₂₇R₁₂₈, wherein phenyl, benzyl or heteroaryl may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₁₂₇ is hydrogen, C₁-C₆alkyl, C₃-C₆alkenyl or C₃-C₆alkynyl or phenyl which may be substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₁₂₈ is hydrogen, C₁-C₆alkyl, C₃-C₆alkenyl or C₃-C₆alkynyl;
or R₁₂₇ together with R₁₂₈ and the respective N atom to which they are bonded form a carbocyclic 3- to 6-membered ring which may be interrupted by oxygen or by sulfur and/or substituted once, twice or three times by halogen, C₁-C₄alkyl, C₁-C₄haloalkyl, C₁-C₃alkoxy, C₁-C₃haloalkoxy, C₁-C₃alkylthio, C₁-C₃alkylsulfinyl, C₁-C₃alkylsulfonyl, C₁-C₃haloalkylthio, cyano, nitro, C₁-C₄alkoxycarbonyl or by C₁-C₄alkylcarbonylamino;
R₃ is hydroxy, O⁻M⁺ wherein M⁺ is a metal cation or an ammonium cation, or is halogen or S(O)ₚR₈₀, wherein
p is 0, 1 or 2;
R₈₀ is C₁-C₁₂alkyl, C₂-C₁₂alkenyl, C₂-C₁₂alkynyl, C₃-C₁₂allenyl, C₃-C₁₂cycloalkyl or C₅-C₁₂-cycloalkenyl;
or R₈₀ is R₁₂₁—C₁-C₁₂alkylene or R₁₂₂—C₂-C₁₂alkenylene, wherein the alkylene or alkenylene chain may be interrupted by —O—, —S—, —S(O)—, —SO₂— or by —C(O)— and/or substituted from one to five times by R₁₂₃;
or R₈₀ is phenyl which may be substituted once, twice, three times, four times or five times by R₁₂₄;
R₁₂₁ and R₁₂₂ are each independently of the other halogen, cyano, rhodano, hydroxy, C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, C₂-C₆alkenylthio, C₂-C₆alkynylthio, C₁-C₆alkylsulfonyloxy, phenylsulfonyloxy, C₁-C₆-alkylcarbonyloxy, benzoyloxy, C₁-C₄alkoxycarbonyloxy, C₁-C₆alkylcarbonyl, C₁-C₄alkoxycarbonyl, benzoyl, aminocarbonyl, C₁-C₄alkylaminocarbonyl, C₃-C₆cycloalkyl, phenyl, phenoxy, phenylthio, phenylsulfinyl or phenylsulfonyl, wherein the phenyl-containing groups may in turn be substituted once, twice or three times by halogen, C₁-C₃alkyl, C₁-C₃haloalkyl, hydroxy, C₁-C₃alkoxy, C₁-C₃haloalkoxy, cyano or by nitro;
R₁₂₃ is hydroxy, halogen, C₁-C₆alkyl, C₁-C₆alkoxy, C₁-C₆alkylthio, C₁-C₆alkylsulfinyl, C₁-C₆alkylsulfonyl, cyano, carbamoyl, carboxy, C₁-C₄alkoxycarbonyl or phenyl, wherein phenyl may be substituted once, twice or three times by hydrogen, C₁-C₄alkyl, C₁-C₄haloalkyl, C₃-C₄alkenyl, C₃-C₄alkynyl or by C₁-C₄alkoxy;
R₁₂₄ is halogen, C₁-C₃alkyl, C₁-C₃haloalkyl, hydroxy, C₁-C₃alkoxy, C₁-C₃haloalkoxy, cyano or nitro;
A₁ is —C(R₁₁₂R₁₁₃)— or —NR₁₁₄—;
A₂ is —C(R₁₁₅R₁₁₆)ₘ—, —C(═O)—, —O—, —NR₁₁₇— or —S(O)_{q}—;
A₃ is —C(R₁₁₈R₁₁₉)— or —NR₁₂₀—;
with the proviso that A₂ is other than —O— or —S(O)_{q}— when A₁ is —NR₁₁₄— and/or A₃ is —NR₁₂₀;
R₁₁₂ and R₁₁₈ are each independently of the other hydrogen, C₁-C₄alkyl, C₂-C₄alkenyl, C₂-C₄alkynyl, C₁-C₄alkylthio, C₁-C₄alkylsulfinyl, C₁-C₄alkylsulfonyl, C₁-C₄alkoxycarbonyl, hydroxy, C₁-C₄alkoxy, C₃-C₄alkenyloxy, C₃-C₄alkynyloxy, hydroxy-C₁-C₄alkyl, C₁-C₄alkylsulfonyloxy-C₁-C₄alkyl, halogen, cyano or nitro;
R₁₁₃ and R₁₁₉ are each independently of the other hydrogen, C₁-C₄alkyl or C₁-C₄alkylthio, C₁-C₄alkylsulfinyl or C₁-C₄alkylsulfonyl;
or R₁₁₃ together with R₁₁₂ and/or R₁₁₉ together with R₁₁₈ form a C₂-C₅alkylene chain which may be interrupted by —O—, —C(O)O— or by —S(O)ᵣ—;
R₁₁₄ and R₁₂₀ are each independently of the other hydrogen, C₁-C₄alkyl, C₁-C₄haloalkyl, C₃-C₄alkenyl, C₃-C₄alkynyl or C₁-C₄alkoxy;
R₁₁₅ is hydrogen, hydroxy, C₁-C₄alkyl, C₁-C₄haloalkyl, C₁-C₃hydroxyalkyl, C₁-C₄alkoxy-C₁-C₃alkyl, C₁-C₄alkylthio-C₁-C₃alkyl, C₁-C₄alkylcarbonyloxy-C₁-C₃alkyl, C₁-C₄alkylsulfonyloxy-C₁-C₃alkyl, tosyloxy-C₁-C₃alkyl, di(C₁-C₄alkoxy)C₁-C₃alkyl, C₁-C₄alkoxycarbonyl, formyl, C₃-C₅oxacycloalkyl, C₃-C₅thiacycloalkyl, C₃-C₄dioxacycloalkyl, C₃-C₄dithiacycloalkyl, C₃-C₄-oxathiacycloalkyl, C₁-C₄alkoxyiminomethyl, cyano, carbamoyl, C₁-C₄alkylaminocarbonyl or di(C₁-C₄alkyl)aminocarbonyl;
or R₁₁₅ together with R₁₁₂ or R₁₁₃ or R₁₁₄ or R₁₁₆ or R₁₁₈ or R₁₁₉ or R₁₂₀ or, when m is 2, also with a second R₁₁₅ form a C₁-C₄alkylene bridge;
R₁₁₆ is hydrogen, C₁-C₃alkyl or C₁-C₃haloalkyl;
R₁₁₇ is hydrogen, C₁-C₃alkyl, C₁-C₃haloalkyl, C₁-C₄alkoxycarbonyl, C₁-C₄alkylcarbonyl or di(C₁-C₄alkyl)aminocarbonyl;
m is 1 or 2; and
q and r are each independently of the other 0, 1 or 2;
and also to agronomically acceptable salts, tautomers, isomers and enantiomers of those compounds.

2. A compound of formula II 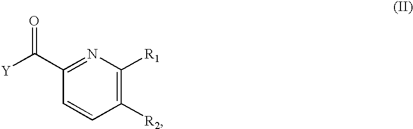
wherein R₁ and R₂ are as defined for formula I in claim 1 and Y is C₁-C₄alkoxy, benzyloxy, hydroxy, fluorine, chlorine, bromine, cyano or phenoxy which may be substituted by an electron-withdrawing group.

3. A herbicidal composition which, besides comprising formulation adjuvants, comprises a herbicidally effective amount of compound of formula I.

4. A method of controlling grasses and weeds in crops of useful plants, which comprises applying a herbicidally effective amount of a compound of formula I or of a composition comprising such a compound to the plants or the locus thereof.
